(19) United States
(12) Patent Application Publication
(10) Pub. No.: US 2023/0286955 A1
(43) Pub. Date: Sep. 14, 2023

(54) INHIBITORS OF THEM1

(71) Applicants: Cornell University, Ithaca, NY (US); Tri-Institutional Therapeutics Discovery Institute, Inc., New York, NY (US)

(72) Inventors: David E. Cohen, New York, NY (US); Christopher Krumm, New York, NY (US); John Ginn, New York, NY (US)

(21) Appl. No.: 18/116,625

(22) Filed: Mar. 2, 2023

Related U.S. Application Data
(60) Provisional application No. 63/315,799, filed on Mar. 2, 2022.

(51) Int. Cl. C07D 403/06; A61P 1/16; A61P 3/10

(52) U.S. Cl. CPC C07D 403/06; A61P 1/16; A61P 3/10

(57) ABSTRACT

The present disclosure relates to compounds that inhibit Them1. The disclosure further relates to methods of treating a metabolic disease in a subject in need thereof.

### RELATED APPLICATIONS

This patent application claims the benefit of U.S. Provisional Patent Application No. 63/315,799, filed on Mar. 2, 2022, which is hereby incorporated by reference in its entirety.

### STATEMENT REGARDING FEDERALLY SPONSORED RESEARCH

This invention was made with government support under Grants R37 DK048873, R01 DK103046, and R01 DK056626 awarded by the National Institutes of Health. The government has certain rights in the invention.

### BACKGROUND

Obesity associated metabolic disorders including type 2 diabetes and non-alcoholic fatty liver disease (NAFLD) are common multigenic disorders that manifest in response to overnutrition (Blüher, 2019; Younossi et al., 2016). These metabolic abnormalities characterized by central adiposity, insulin resistance, hypertriglyceridemia and high blood pressure are rising worldwide at an alarming rate. Despite the increasing prevalence of these disorders, current therapy options remain limited. Although healthy lifestyle changes including weight loss are effective for the management of these disorders, they have not generally been met with success in the clinic. Brown (BAT) and beige (BeAT) adipose tissues in rodents and humans are rich in mitochondria and promote energy expenditure by non-shivering thermogenesis, a process whereby large quantities of caloric energy are converted to heat. It is now appreciated that BAT and BeAT also play key roles in human energy expenditure, and that therapeutic interventions to increase the activity and mass of BAT and BeAT are predicted to reduce obesity and mitigate its associated disorders including type 2 diabetes and NAFLD.

Following uptake into cells, fatty acids are activated by esterification to coenzyme A (CoA) molecules by acyl-CoA synthetases. Fatty acyl-CoAs may then be oxidized or incorporated into complex lipids. Acots are divided into two subfamilies consisting of type I isoforms (i.e., 1-6), which contain a conserved α/β hydrolase catalytic domain as well as high sequence homology, and type II isoforms (i.e., 7-15), which share common structural features including a structural ‘HotDog domain’ motif, but exhibit low sequence homology (Tillander et al., 2017). Mammalian cells also express Acots, which hydrolyze fatty acyl-CoAs to form fatty acids and CoA. Emerging data implicate Acots in the pathogenesis of obesity associate metabolic disorders including type 2 diabetes and NAFLD (Tillander et al., 2017).

### SUMMARY OF THE INVENTION

In certain embodiments, the present disclosure provides compounds represented by formula (I):

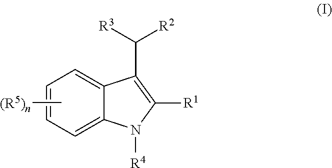

and pharmaceutically acceptable salts thereof, wherein:
R¹ is H, alkyl, alkenyl, alkynyl, aryl, heteroaryl, cycloalkyl, heterocycloalkyl, halogen, cyano, hydroxy, alkoxy, amino, acylamino, amide, acyl, acyloxy, ester, sulfonamide, or sulfone;
R² is aryl, heteroaryl, cycloalkyl, cycloalkenyl, or heterocycloalkyl;
R³ is aryl, heteroaryl, cycloalkyl, cycloalkenyl, or heterocycloalkyl;
R⁴ is H, alkyl, alkenyl, or alkynyl,
R⁵ is halogen, alkyl, or cycloalkyl, and
n is 0-4.

In certain aspects, the present disclosure provides pharmaceutical compositions comprising a compound provided herein and a pharmaceutically acceptable excipient.

In certain aspects, the present disclosure provides methods of treating metabolic diseases, such as non-alcoholic fatty liver disease, comprising administering to a patient in need thereof a compound or composition of the disclosure.

In certain aspects, the present disclosure provides methods of treating cardiovascular diseases, comprising administering to a patient in need thereof a compound or composition of the disclosure.

### BRIEF DESCRIPTION OF THE DRAWINGS

FIG. 1 shows inhibition and binding of Them1 by START domain-dependent compounds. Structural features of U series small molecules that selectively inhibit Them1 activity through the START domain. IC₅₀ values for inhibition of Acot activities are shown. Binding affinities as quantified by K_{d} values are shown. Data represents the mean (s.e.m.) of triplicate determinations. Where not visible, standard error bars are contained within the symbol sizes.

FIG. 2 shows inflection temperature (Ti) curves of recombinant His-tagged human Them1 in the presence of compound U1. Values of changes in inflection temperature (Ti ° C.) of Them1 in the presence of compound U1.

FIGS. 3A-C show small molecule inhibitors increase OCR in primary brown adipocytes. FIG. 3A shows the experimental design to test Them1 small molecule inhibitors in cultured primary brown adipocytes. FIGS. 3B-C show OCR values in primary brown adipocytes following stimulation with norepinephrine (NE) and treatment with the following compounds at 10 μM: (FIG. 3B) U1; (FIG. 3C) U2. The bar graphs in each panel represent relative NE-stimulated values of area under the curve (AUC). Data in the graphs represent the mean±s.e.m. of 2 to 3 independent experiments. Where not visible, standard error bars are contained within the symbol sizes. ***P<0.001; Control (DMSO) vs. A-Them1Tg (DMSO); ^{§§§}P<0.001; Control (DMSO) vs. A-Them1Tg (U2).

FIGS. 4A-H show recombinant Acot purification. Acot isoform panels: Left panels, recombinant Acot isoform enzymes including type I [Acot1, Acot2] and type II [Acot9, Acot12 and Acot13], as well as a truncated Them1 either containing only the 2 thioesterase domains but lacking the START domain (Them1-ΔSTART) or the START domain but lacking the 2 thioesterase domains (START) were purified from Shuffle T7 or BL21 (DE3) E. coli competent cells on a HisTrap HP column unless otherwise specified. Recombinant START was further purified using a Superdex 75 column. Recombinant Acot9 was purified on a MBPTrap HP column. Upper right panel, SDS-PAGE of recombinant Acot isoform enzymes. Right panel, saturation curves of recombinant Acot isoform enzymes and myristoyl (C14)-CoA reactions (black dots) were used to determine steady state kinetic constants. Where not visible, standard error bars are contained within the symbol sizes. FIG. 4H shows recombinant Acot steady state kinetic constants. Table, steady state kinetic constants (Kₘ, Vₘₐₓ, K_{cat} and K_{cat}/Kₘ) were determined as described in the text. Data represents the mean (s.e.m.) of triplicate determinations.

FIGS. 5A-B show reversibility of inhibition by small molecules targeting Them1 activity. All reactions were performed in 384-well microplates at 22° C. Pre-dialysis reactions were performed with recombinant His-tagged human Them1 (Them1) and myristoyl (C14)-CoA. Reactions subjected to an overnight dialysis were incubated with C14-CoA. (FIG. 5A) Time course of Pre-(white dots) and Post-(black dots) dialysis reactions for the indicated timepoints. (FIG. 5B) Pre- and Post-reactions consisting of compounds U1 or Ebselen [(−) control]. Each graph is representative of 4 independent experiments. Data represents the mean±s.e.m. of triplicate determinations. Where not visible, standard error bars are contained within the symbol sizes. ***, P<0.001; Pre-(U1) vs. Post-(U1).

FIGS. 6A-B show (FIG. 6A) Left panels, recombinant His-tagged mouse Them1 was purified from BL21 (DE3) E. coli competent cells on a HisTrap HP column. Upper right panel, SDS-PAGE of recombinant mouse Them1. Right panel, saturation curve of recombinant mouse Them1 (125 nM) and myristoyl (C14)-CoA reactions (black dots) was used to determine steady state kinetic constants. (FIG. 6B) All reactions were performed with either recombinant mouse Them1 or recombinant human Them1. Acot activities quantified as IC₅₀ values are shown. Data represents the mean±s.e.m. of triplicate determinations. Where not visible, standard error bars are contained within the symbol sizes.

FIG. 7A shows generation of conditional transgenic tissue-specific Them1 overexpression (wild type) mice was previously described with modifications (Madisen et al., 2010). A cDNA encoding mouse Them1 was fused to a C-terminal FLAG-tag (FLAG) as a marker for Cre recombination and cloned into a Rosa26 expression vector consisting of a CMV promoter, a STOP cassette flanked by loxP sites and a polyA tail (pA) to generate conditional transgenic tissue-specific Them1 overexpression (wild type) mice. FIG. 7B shows conditional transgenic adipose tissue-specific Them1 overexpression (A-Them1Tg) mice were generated by crossing wild type mice to transgenic mice expressing Cre recombinase driven by the adiponectin gene promoter on a congenic C57/BL6 background. FIG. 7C shows mice with liver-specific Them1 overexpression (L-Them1Tg) and wild type controls, along with Them1 knockout (Them1^{-/-}) controls were generated by intravenously injecting 6- to 14-w old wild type, C57BL/6 and Them1^{-/-} mice (Zhang et al., 2012), respectively, with adeno-associated virus 8 (AAV8) expressing Cre recombinase driven by the human thyroid hormone-binding globulin (TBG) promoter (AAV8.TBG.Cre). Adiponectin- and TBG-Cre mediated recombination resulted in excision of the STOP cassette to bring Them1 under control of the CMV promoter in adipose tissue and hepatocytes, respectively. FIG. 7D shows relative mRNA expression level of mouse Them1 (Them1) in white adipose tissue (WAT), brown adipose tissue (BAT) and kidney extracts were analyzed by quantitative real-time PCR. FIG. 7E shows relative protein expression levels of Them1 and FLAG in brain, heart, lung, WAT, skeletal muscle (muscle), kidney and BAT were analyzed by immunoblotting with total protein utilized to control for unequal loading. Control, n=5; A-Them1Tg, n=5. FIG. 7F shows relative mRNA expression level of Them1 in total cellular extracts from primary brown adipocytes were analyzed by quantitative real-time PCR. FIG. 7G shows relative protein expression levels of Them1 and FLAG in primary brown adipocytes were analyzed by immunoblotting with Hsp90 utilized to control for unequal loading. Control, n=6; A-Them1Tg, n=6. Data represents the mean (SEM). **, P<0.01; ***, P<0.001; Control vs. A-Them1Tg. FIG. 7H shows relative protein expression levels of Them1 and FLAG in primary hepatocytes cultured from L-Them1Tg and Them1^{-/-} mice were analyzed by immunoblotting with Hsp90 utilized to control for unequal loading. L-Them1Tg, n=6; Them1^{-/-}, n=6. Data represent the mean±s.e.m. **, P<0.01; ***, P<0.001; wild type vs. A-Them1Tg.

FIGS. 8A-C show effect of small molecule inhibitors OCR values in primary brown adipocytes. OCR values in primary brown adipocytes cultured from wild type and transgenic adipose tissue-specific Them1 overexpression (A-Them1Tg) mice following stimulation with norepinephrine (NE; 1 μM), and treatment for 30 min with the following compounds: (FIG. 8A) U1at 20 or 30 μM, (FIG. 8B) U3 and U4 at 10 μM and (FIG. 8C) U5 at 10 μM. The bar graphs in each panel represent relative NE-stimulated values of area under the curve (AUC). Data in the graphs represent the mean±s.e.m. of 2 to 3 independent experiments. Where not visible, standard error bars are contained within the symbol sizes. ***, P<0.001; Control (DMSO) vs. A-Them1Tg (DMSO); ##, P<0.01; Control (DMSO) vs. A-Them1Tg (U3).

FIGS. 9A-F shows that Them1 inhibitors increase fatty acid oxidation rates and suppress glucose production in cultured primary hepatocytes. FIG. 9A-C illustrate the experimental design to test Them1 small molecule inhibitors in primary hepatocytes cultured from wild type, transgenic liver-specific Them1 overexpression (L-Them1Tg) and Them1 knockout (Them1^{-/-}) mice. FIGS. 9D-E display oxygen consumption rate (OCR) values in primary hepatocytes following stimulation with palmitate conjugated with fatty acid-free BSA (300 μM) and treatment with compound U1 (33 μM). The bar graphs in each panel represent relative values of area under the curve (AUC) following stimulation with palmitate. Data in the graphs represent the mean±s.e.m. of 2 independent experiments. Where not visible, standard error bars are contained within the symbol sizes. *P<0.05; wild type (DMSO) vs. L-Them1Tg (DMSO). FIG. 9F shows relative glucose production compared to wild type controls in serum-starved primary hepatocytes were determined by appearance of glucose in media following addition of compound U1 (33 μM), and pyruvate (2 mM) and lactate (20 mM) as gluconeogenic substrates. Data in the graphs represent the mean±s.e.m. of 2 independent experiments. ***P<0.001; wild type (DMSO) vs. L-Them1Tg (DMSO); P=0.08; L-Them1Tg (DMSO) vs. L-Them1Tg (U1).

(FIGS. 10A-D) Compound U1 and structural derivatives selectively bind the Them1 START domain. Binding interactions were quantified as K_{d} values by microscale thermophoresis using recombinant His-tagged proteins labeled with Monolith RED-Tris-NTA (100 nM) (i.e. (FIG. 10A) full-length Them1, (FIG. 10B) a truncated Them1 either containing only the START domain but lacking the 2 thioesterase domains [START], (FIG. 10C) only the 2 thioesterase domains but lacking the START domain [Them1-ΔSTART] or (FIG. 10D) full-length human Acot12 [Acot12]) and compounds. Data represent the mean (s.e.m.) of triplicate determinations. Where not visible, error bars are contained within the symbol sizes.

FIGS. 11A-E show small molecule inhibitors increase oxygen consumption rates (OCR) in cultured primary brown adipocytes. OCR values in primary brown adipocytes cultured from wild type and transgenic adipose tissue-specific Them1 overexpression (A-Them1Tg) mice following stimulation with norepinephrine (NE; 1 μM), and treatment for 30 min with the following compounds: (FIG. 11A) U6 and U9, (FIG. 11B) U11 (FIG. 11C) U12, (FIG. 11D) U14 and U15 and (FIG. 11E) U8. The bar graphs in each panel represent relative values of area under the curve (AUC) following NE stimulation. Data in the graphs represent the mean±s.e.m. of 2 independent experiments. Where not visible, standard error bars are contained within the symbol sizes. ***, P<0.001; wild type (DMSO) vs. A-Them1Tg (DMSO); #, P<0.01; wild type (DMSO) vs. A-Them1Tg (U14); ‡‡‡, P<0.001; wild type (DMSO) vs. A-Them1Tg (U15); §§§ , P<0.001; wild type (DMSO) vs. A-Them1Tg (U8; 30 μ4M); †††, P<0.001; wild type (DMSO) vs. A-Them1Tg (U8; 60 μM). FIG. 12 shows pre-dialysis reactions were performed with recombinant His-tagged human Them1 (Them1), myristoyl (C14)-CoA and synthesized compound U1. Reactions subjected to an overnight dialysis were incubated with C14-CoA. Data in the graph is representative of 2 independent experiments. Data represent the mean±s.e.m. of triplicate determinations. Where not visible, standard error bars are contained within the symbol sizes. ***, P<0.001; Pre-(U1) vs. Post-(U1).

FIG. 13 Pre-dialysis reactions were performed with recombinant His-tagged human Them1 (Them1), myristoyl (C14)-CoA as well as compound U1structural derivatives or Ebselen [(−) control. Reactions subjected to an overnight dialysis were incubated with C14-CoA. Data in the graph is representative of 2 independent experiments. Data represent the mean±s.e.m. of triplicate determinations. Where not visible, standard error bars are contained within the symbol sizes. ***, P<0.001; Pre-(Compound) vs. Post-(Compound).

FIG. 14. Assessment of the solubility of compound U1 and its structural derivatives. Critical aggregation concentration values of compound U1 and its structural derivatives were determined by dynamic light scattering using serial 2-fold dilutions of compounds (250 μM-31.5 μM) in PBS. Data represent triplicate determinations for 2 independent experiments.

FIG. 15 Cytotoxicity of compound U1 and its structural derivatives. Cytotoxicity of small molecule inhibitors with START-domain binding in primary brown adipocytes. Cell viability for compound U1 following a 48 h treatment period in primary brown adipocytes cultured from wild type mice. Lethal concentrations (LC₅₀) were >125 μM for compounds U1-U17. Tamoxifen served as a (+) control for the assay. Data in the graphs represent the mean of 2 independent experiments. Where not visible, standard error bars are contained within the symbol sizes.

### DETAILED DESCRIPTION OF THE INVENTION

Thioesterase superfamily member 1 (Them1) [synonyms: Acot11 and steroidogenic acute regulatory protein-related lipid transfer (START) domain 14 (StarD14)] is a long-chain type II Acot that is highly expressed in thermogenic adipose tissue including BAT and BeAT, and is robustly upregulated in response to cold exposure and downregulated by warm temperatures (Zhang et al., 2012). In contrast to expectations that Them1 would function to promote thermogenesis, it was observed increases in energy expenditure in Them1^{-/-} mice. When challenged with a high fat diet (HFD), these mice exhibited improved glucose and lipid homeostasis and were protected against diet-induced obesity, insulin resistance and hepatic steatosis (Zhang et al., 2012). In addition to limiting caloric consumption by thermogenesis, Them1 is strongly upregulated in liver, WAT and BeAT in response to high-fat feeding, where it contributes toward the pathogenesis of obesity and related disorders including type 2 diabetes and NAFLD (Desai et al., 2018; Zhang et al., 2012).

### Them1 Conserves Energy by Suppressing Thermogenesis in BAT

During cold exposure, norepinephrine is released by the sympathetic nervous system and stimulates β₃-adrenergic receptors in BAT (Cannon and Nedergaard, 2004). This leads to the hydrolysis of lipid droplet triglycerides to release fatty acids (Holm, 2003; Sztalryd and Kimmel, 2014). Conversion to fatty acyl-CoAs by long-chain acyl-CoA synthetase 1 channels fatty acids into the mitochondria (Ellis et al., 2010), where β-oxidation leads to heat production when uncoupling protein 1 dissipates the proton gradient produced by the electron transport chain. Activation of BAT also leads to marked transcriptional upregulation of Them1. By converting fatty acyl-CoAs to free fatty acids plus CoA, Them1 limits trafficking of fatty acids from lipid droplets to mitochondria (Okada et al., 2016; Zhang et al., 2012), thereby reducing rates of fatty acid oxidation and thermogenesis. These data are indicative that Them1 functions in BAT to conserve energy.

1. Them1 reduces thermogenesis in BAT. Increased energy expenditure through thermogenesis in BAT is a major mechanism by which Them1^{-/-} mice are protected against diet-induced weight gain, insulin resistance and NAFLD (Zhang et al., 2012). Although energy conservation is critical when nutrients are scarce, this mechanism becomes maladaptive during overnutrition.

2. Them1 limits metabolic benefits of BeAT formation. The integrated stress response (ISR) is a cellular signaling system that helps restore homeostasis following exposure to environmental stresses (Costa-Mattioli and Walter, 2020). Activation of the ISR in liver by HFD feeding leads to the upregulation of hepatic expression of fibroblast growth factor 21 (FGF21) production, a hepatokine with protective effects that include increased BeAT formation (i.e. beiging of WAT). ISR activation by liver-specific deletion of constitutive repressor of eIF2α phosphorylation (CReP) was sufficient to mitigate obesity, insulin resistance and hepatic steatosis in HFD-fed mice (Xu et al., 2018). This was attributable to increased thermogenesis secondary to FGF21-mediated BeAT formation. It was further shown that Them1 was markedly upregulated in BeAT of L-CReP^{-/-} mice, and that Them1 functions maladaptively to attenuate thermogenesis and offset the positive effects of FGF21. These findings demonstrate that Them1 contributes to NAFLD by limiting ISR-mediated activation of thermogenesis.

3. HFD-induced Them1 upregulation in liver promotes hepatic steatosis and excess glucose production. HFD feeding strongly upregulates Them1 expression in mouse liver (Desai et al., 2018; Ellis et al., 2015). Relative to HFD-fed controls (Them1^{+/+}) and control Them1^{-/-} mice, mice expressing Them1 exclusively in the liver (L-Them1^{-/-}Tg) mice exhibited no changes in growth or energy expenditure (Desai et al., 2018). However, histological analysis revealed steatosis in HFD-fed L-Them1^{-/-}Tg mice and elevated hepatic triglyceride concentrations relative to Them1^{-/-} mice. This was primarily attributable to reduced rates of fatty acid oxidation. Whereas glucose and insulin tolerance did not differ, hepatic glucose production was increased in L-Them^{-/-}Tg mice. This was in keeping with the upregulation of the mRNA levels of the gluconeogenic genes phosphoenolpyruvate carboxykinase and glucose-6-phosphatase in these mice. These findings indicate that Them1 plays a pathogenic role in HFD-induced steatosis and excess hepatic glucose production.

4. HFD-induced upregulation of Them1 in WAT promotes inflammation. WAT inflammation contributes to insulin resistance and hepatic steatosis in NAFLD (Loomba et al., 2021). HFD feeding upregulates Them1 expression in WAT (Zhang et al., 2012). Macrophages accumulated in WAT from Them1^{+/+} but not Them1^{-/-} mice, which exhibited attenuated plasma cytokine concentrations in response to HFD feeding. Similarly, the expression of mRNAs encoding pro-inflammatory genes were reduced in WAT of Them1^{-/-} mice. It was further demonstrated that Them1 expression in white adipocytes could influence macrophage activation. Induction of inflammatory genes were attenuated when bone marrow-derived macrophages were cultured in media from Them1^{-/-} compared with Them1^{+/+} white adipocytes. These data signify a pathogenic role of Them1 in HFD-induced WAT inflammation.

### Evidence for the Relevance of Them1 to Human NAFLD

In an earlier study, Them1 was mapped to syntenic regions of human chromosome 1 and mouse chromosome 4 that are associated with adiposity and diet-induced obesity, respectively (Adams et al., 2001). However, to date, mutations in Them1 have not been related to human metabolic disorders. A coding sequence variant was identified by whole-exome sequencing in a single 65 year-old male patient with lipid storage myopathy (Goh et al., 2018), but the molecular pathogenesis was instead attributed to compound heterozygous mutations in electron transfer flavoprotein dehydrogenase, which are known to cause this disorder in the absence of mutations in Them1. Although genotype-phenotype correlations are not yet available, a variety of other Them1 single nucleotide polymorphisms have been reported, including in the coding region (ensemble.org). Support for a pathogenic role of Them1 in human NAFLD can be gleaned from re-analyses of unbiased gene expression studies of human tissues (Desai et al., 2018; Okada et al., 2016; Zhang et al., 2012; unpublished findings). Upregulation of Them1 in human BAT in response to cold exposure is consistent with observations in mice (Adams et al., 2001; Zhang et al., 2012). Upregulation of Them1 in livers of patients with NAFLD relative to lean subjects is in keeping with its maladaptive role in promoting steatosis and hepatic glucose production (Desai et al., 2018), and upregulation in livers of non-alcoholic steatohepatitis relative to NAFLD patients is consistent with its contribution to hepatic inflammation (Kazankov et al., 2019; Zhang et al., 2012). Increased expression of Them1 in both WAT of obese relative to lean human subjects and visceral WAT relative to subcutaneous WAT suggest its contribution to WAT inflammation (Kazankov et al., 2019; Zhang et al., 2012).

### Molecular Properties of Them1 That Support Targeting by Small Molecules

Acots vary in their substrate specificities for molecular species (i.e., chain lengths and saturation) of fatty acyl-CoAs, often exhibiting overlap with other isoforms (Hunt et al., 2005; Tillander et al., 2017). Them1 hydrolyzes a range of fatty acyl-CoAs but exhibits a relative preference for long-chain species (Han and Cohen, 2012). Them1 is enzymatically active as a homotrimer of protomers characterized by a central assembly of thioesterase domains with protruding START domains. It is one of two Acots that comprises tandem N-terminal thioesterase domains fused to a C-terminal START domain. The helix-grip fold structure of the START domain forms a hydrophobic tunnel and accommodates a single lipid molecule (Schrick et al., 2004). START domains generally reside at the C-terminus of multidomain proteins and function as lipid sensors, whereby lipid binding regulates the activity of the thioesterase domains. Insights gleaned from the Them1 START domain structure implied flexibility of the hinge region between the thioesterase and START domains, which likely contributes to lipid-mediated regulation. Indeed, the Them1 START domain regulates the enzymatic domains in response to binding long-chain fatty acids, as well as lysophosphatidylcholines, which activate and suppress acyl-CoA thioesterase activity, respectively (Han and Cohen, 2012; Tillman et al., 2020). This was confirmed in cell culture studies by increased rates of fatty acid oxidation following the addition of lysophosphatidylcholines to the medium following transduction with Them1 relative to a truncated Them1 (Them1l-ΔSTART) lacking the START domain.

Mice with genetic ablation of Them1 (Them1^{-/-}) exhibit increased thermogenesis and are resistant to diet-induced weight gain and NAFLD, indicating that Them1 functions biologically to conserve energy, even when doing so becomes pathogenic (Zhang et al., 2012). Mechanisms include reduced rates of fatty acid oxidation in BAT and BeAT, sequestration of triglycerides in liver, excess hepatic glucose production and increased inflammation in WAT.

Support for a pathogenic role of Them1 in human NAFLD can be gleaned from re-analyses of unbiased gene expression studies of human tissues (Desai et al., 2018; Okada et al., 2016; Zhang et al., 2012; unpublished findings). Upregulation of Them1 in human BAT in response to cold exposure is consistent with observations in mice (Adams et al., 2001; Zhang et al., 2012). Upregulation of Them1 in livers of patients with NAFLD relative to lean subjects is in keeping with its maladaptive role in promoting steatosis and hepatic glucose production (Desai et al., 2018), and upregulation in livers of non-alcoholic steatohepatitis relative to NAFLD patients is consistent with its contribution to hepatic inflammation (Kazankov et al., 2019; Zhang et al., 2012). Increased expression of Them1 in both WAT of obese relative to lean human subjects and visceral WAT relative to subcutaneous WAT suggest its contribution to WAT inflammation (Kazankov et al., 2019; Zhang et al., 2012).

### Compounds

In certain aspects, the current disclosure provides compounds of formula (I)

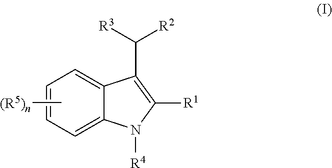

or a pharmaceutically acceptable salt thereof, wherein:
R¹ is H, alkyl, alkenyl, alkynyl, aryl, heteroaryl, cycloalkyl, heterocycloalkyl, halogen, cyano, hydroxy, alkoxy, amino, acylamino, amide, acyl, acyloxy, carboxy, ester, sulfonamide, sulfone;
R² is aryl, heteroaryl, cycloalkyl, cycloalkenyl, or heterocycloalkyl;
R³ is aryl, heteroaryl, cycloalkyl, cycloalkenyl, or heterocycloalkyl;
R⁴ is H, alkyl, alkenyl, or alkynyl,
R⁵ is halogen, alkyl, or cycloalkyl, and
n is 0-4.

In certain preferred embodiments, n is 0. In certain embodiments, n is 1-4. In certain preferred embodiments, n is 1 or 2.

In certain embodiments, the compound is of formula (II):

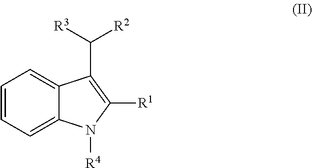

or a pharmaceutically acceptable salt thereof.

In certain embodiments R² is heterocycloalkyl, e.g., a heterocycloalkyl that comprises at least one nitrogen atom. In certain embodiments, the heterocycloalkyl has one nitrogen atom and in other embodiments the heterocycloalkyl has two nitrogen atoms.

In certain embodiments, the compound is of formula (III):

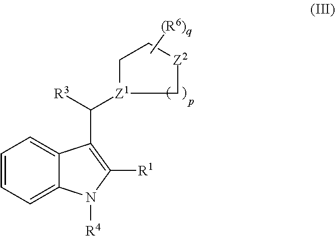

or a pharmaceutically acceptable salt thereof, wherein:
Z¹ is N or CR^{a};
Z² is O, NR^{b} or CR^{c}R^{d};
each R^{a}, R^{b}, R^{c}, and R^{d} is independently H, alkyl, alkenyl, alkynyl, aryl, heteroaryl, cycloalkyl, heterocycloalkyl, halogen, cyano, hydroxy, alkoxy, amino, acylamino, amide, acyl, acyloxy, ester, sulfonamide, sulfone;
each R⁶ is alkyl, alkenyl, alkynyl, aryl, heteroaryl, cycloalkyl, heterocycloalkyl, halogen, cyano, hydroxy, alkoxy, amino, acylamino, amide, acyl, acyloxy, ester, sulfonamide, sulfone;
p is 1 or 2; and
q is 0 to 4.

In certain embodiments, Z¹ is CH. In alternative embodiments Z¹ is N.

In certain embodiments Z² is CR^{c}R^{d}. In alternative embodiments, Z² is O.

In certain embodiments, Z2 is NR^{b}. R^{b} in NR^{b} can be, for example, hydrogen, alkyl (e.g., C₁-C₆ alkyl), —C(O)alkyl (e.g., —C(O)C₁-C₆ alkyl), or —C(O)₂alkyl (e.g., —C(O)₂C₁-C₆ alkyl). In certain embodiments, R^{b} is substituted with cycloalkyl, heterocycloalkyl, aryl such as phenyl, or heteroaryl. For example R^{b} can be —C(O)C₁-C₆ alkylene-phenyl. In certain embodiments, R^{b} is hydrogen, alkyl (e.g., C₁-C₆ alkyl), —C(O)alkyl (e.g., —C(O)C₁-C₆ alkyl), or —C(O)₂alkyl (e.g., —C(O)₂C₁-C₆ alkyl), wherein the alkyl in C₁-C₆ alkyl, —C(O)C₁-C₆ alkyl, and —C(O)₂C₁-C₆ alkyl can be substituted with cycloalkyl or heterocycloalkyl. In certain embodiments, R^{b} is hydrogen, alkyl (e.g., C₁-C₆ alkyl), —C(O)alkyl (e.g., —C(O)C₁-C₆ alkyl), or —C(O)₂alkyl (e.g., —C(O)₂C₁-C₆ alkyl), wherein the alkyl in C₁-C₆ alkyl and —C(O)C₁-C₆ alkyl can be substituted with cycloalkyl, heterocycloalkyl, aryl such as phenyl, or heteroaryl.

In certain embodiments, q is 0, 1, 2, 3, or 4. In certain embodiments, p is 1 or 2 and q is 0.

In certain embodiments, the compound is of formula (IV)

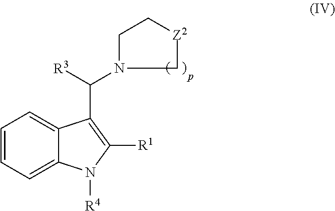

or a pharmaceutically acceptable salt thereof, wherein:
Z² is O, NR^{b} or CR^{c}R^{d};
each of R^{b}, R^{c}, and R^{d} is independently H, —C(O)OC₁-C₆ alkyl, —C(O)C₁-C₆ alkyl, or —C(O)-aryl, wherein C₁-C₆ alkyl is optionally substituted by aryl; and
p is 1 or 2.

In certain embodiments, the compound is of formula (V)

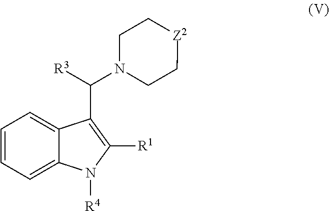

or a pharmaceutically acceptable salt thereof, wherein:
Z² is O, NR^{b} or C(H)R^{c}; and
each of R^{b} and R^{c} is independently H, —C(O)OC₁-C₆ alkyl, —C(O)C₁-C₆ alkyl, or —C(O)-aryl, e.g., wherein each of R^{b} and R^{c} is optionally substituted by aryl.

In certain embodiments, Z² is NR^{b} in formula (IV) or (V). R^{b} in NR^{b} can be, for example, hydrogen, alkyl (e.g., C₁-C₆ alkyl), —C(O)alkyl (e.g., —C(O)C₁-C₆ alkyl), or —C(O)₂alkyl (e.g., —C(O)₂C₁-C₆ alkyl). In certain embodiments, R^{b} is substituted with cycloalkyl, heterocycloalkyl, aryl such as phenyl, or heteroaryl. For example R^{b} can be —C(O)C₁-C₆ alkylene-phenyl. In certain embodiments, R^{b} is hydrogen, alkyl (e.g., C₁-C₆ alkyl), —C(O)alkyl (e.g., —C(O)C₁-C₆ alkyl), or —C(O)₂alkyl (e.g., —C(O)₂C₁-C₆ alkyl), wherein the alkyl in C₁-C₆ alkyl, —C(O)C₁-C₆ alkyl, and —C(O)₂alkyl can be substituted with cycloalkyl or heterocycloalkyl. In certain embodiments, R^{b} is hydrogen, alkyl (e.g., C₁-C₆ alkyl), —C(O)alkyl (e.g., —C(O)C₁-C₆ alkyl), or —C(O)₂alkyl (e.g., —C(O)₂C₁-C₆ alkyl), wherein the alkyl in C₁-C₆ alkyl and —C(O)C₁-C₆ alkyl can be substituted with cycloalkyl, heterocycloalkyl, aryl such as phenyl, or heteroaryl.

In some alternative embodiments, Z² is O in formula (IV) or (V). In still yet some other alternative embodiments, Z² is CR^{c}R^{d} such as C(H)R^{c} in formula (IV) or (V) and R^{c} is an ester (e.g., —C(O)O-alkyl such as —C(O)OC₁-C₆ alkyl).

In certain embodiments, R³ is aryl or heteroaryl (e.g., thiopheneyl). R³ can be substituted with, for example alkyl such as C₁-C₆ alkyl. For example R³ can be a methyl-substituted heteroaryl such as a methyl-substituted thiophene. In certain embodiments, R³ is not phenyl.

In certain embodiments, R¹ is H or alkyl such as C₁-C₆ alkyl. For example, R¹ can be methyl or ethyl. In certain embodiments, R¹ is substituted.

In certain embodiments, R⁴ is H or alkyl such as C₁-C₆ alkyl. For example, R⁴ can be methyl or ethyl. In certain embodiments, R¹ is H.

In certain embodiments, the compound is selected from:

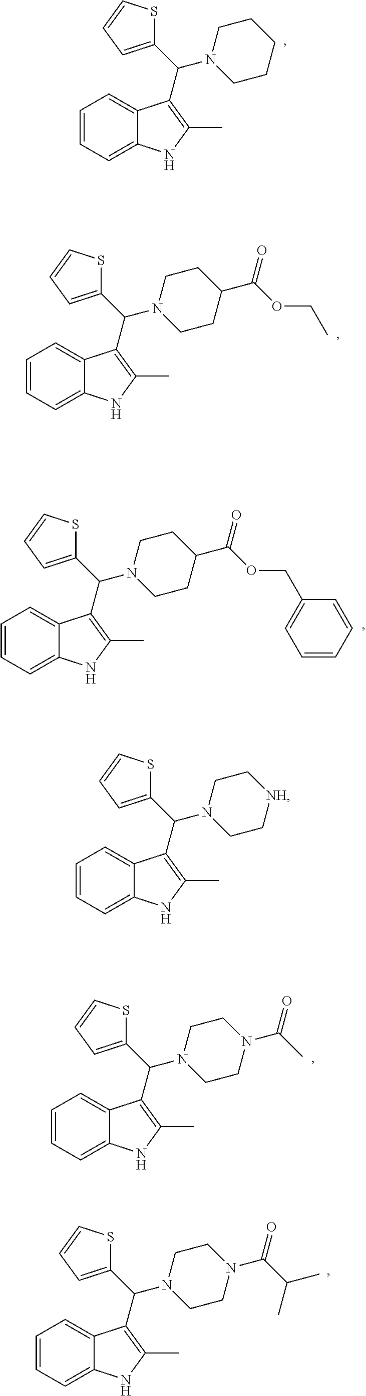 [SMILES: Cc1[nH]c2ccccc2c1C(c1cccs1)N1CCCCC1 ; CCOC(=O)C1CCN(C(c2cccs2)c2c(C)[nH]c3ccccc23)CC1 ; Cc1[nH]c2ccccc2c1C(c1cccs1)N1CCC(C(=O)OCc2ccccc2)CC1 ; Cc1[nH]c2ccccc2c1C(c1cccs1)N1CCNCC1 ; CC(=O)N1CCN(C(c2cccs2)c2c(C)[nH]c3ccccc23)CC1 ; Cc1[nH]c2ccccc2c1C(c1cccs1)N1CCN(C(=O)C(C)C)CC1]

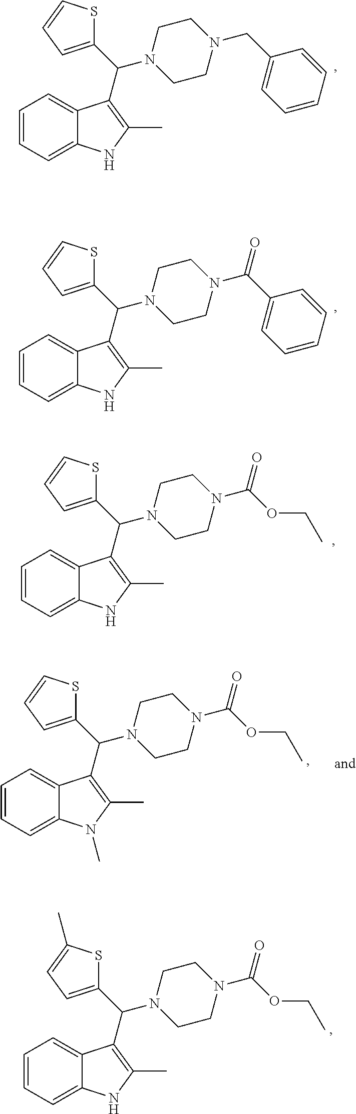 [SMILES: Cc1[nH]c2ccccc2c1C(c1cccs1)N1CCN(Cc2ccccc2)CC1 ; Cc1[nH]c2ccccc2c1C(c1cccs1)N1CCN(C(=O)c2ccccc2)CC1 ; CCOC(=O)N1CCN(C(c2cccs2)c2c(C)[nH]c3ccccc23)CC1 ; CCOC(=O)N1CCN(C(c2cccs2)c2c(C)n(C)c3ccccc23)CC1 ; CCOC(=O)N1CCN(C(c2ccc(C)s2)c2c(C)[nH]c3ccccc23)CC1]

or a pharmaceutically acceptable salt thereof.

In certain embodiments, the compound is not

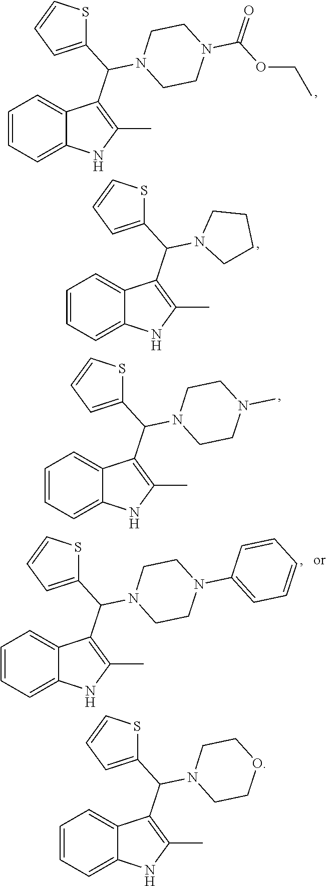

In certain aspects, the present disclosure provides pharmaceutical compositions, comprising the compound of any one of the preceding claims and a pharmaceutically acceptable excipient.

### Pharmaceutical Compositions

The compositions and methods of the present disclosure may be utilized to treat an individual in need thereof. In certain embodiments, the individual is a mammal such as a human, or a non-human mammal. When administered to an animal, such as a human, the composition or the compound is preferably administered as a pharmaceutical composition comprising, for example, a compound of the disclosure and a pharmaceutically acceptable carrier. Pharmaceutically acceptable carriers are well known in the art and include, for example, aqueous solutions such as water or physiologically buffered saline or other solvents or vehicles such as glycols, glycerol, oils such as olive oil, or injectable organic esters. In preferred embodiments, when such pharmaceutical compositions are for human administration, particularly for invasive routes of administration (i.e., routes, such as injection or implantation, that circumvent transport or diffusion through an epithelial barrier), the aqueous solution is pyrogen-free, or substantially pyrogen-free. The excipients can be chosen, for example, to effect delayed release of an agent or to selectively target one or more cells, tissues or organs. The pharmaceutical composition can be in dosage unit form such as tablet, capsule (including sprinkle capsule and gelatin capsule), granule, lyophile for reconstitution, powder, solution, syrup, suppository, injection or the like. The composition can also be present in a transdermal delivery system, e.g., a skin patch. The composition can also be present in a solution suitable for topical administration, such as a lotion, cream, or ointment.

A pharmaceutically acceptable carrier can contain physiologically acceptable agents that act, for example, to stabilize, increase solubility or to increase the absorption of a compound such as a compound of the disclosure. Such physiologically acceptable agents include, for example, carbohydrates, such as glucose, sucrose or dextrans, antioxidants, such as ascorbic acid or glutathione, chelating agents, low molecular weight proteins or other stabilizers or excipients. The choice of a pharmaceutically acceptable carrier, including a physiologically acceptable agent, depends, for example, on the route of administration of the composition. The preparation or pharmaceutical composition can be a self-emulsifying drug delivery system or a self-2microemulsifying drug delivery system. The pharmaceutical composition (preparation) also can be a liposome or other polymer matrix, which can have incorporated therein, for example, a compound of the disclosure. Liposomes, for example, which comprise phospholipids or other lipids, are nontoxic, physiologically acceptable and metabolizable carriers that are relatively simple to make and administer.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically acceptable carrier” as used herein means a pharmaceutically acceptable material, composition or vehicle, such as a liquid or solid filler, diluent, excipient, solvent or encapsulating material. Each carrier must be “acceptable” in the sense of being compatible with the other ingredients of the formulation and not injurious to the patient. Some examples of materials which can serve as pharmaceutically acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

A pharmaceutical composition (or preparation) can be administered to a subject by any of a number of routes of administration including, for example, orally (for example, drenches as in aqueous or non-aqueous solutions or suspensions, tablets, capsules (including sprinkle capsules and gelatin capsules), boluses, powders, granules, pastes for application to the tongue); absorption through the oral mucosa (e.g., sublingually); subcutaneously; transdermally (for example as a patch applied to the skin); and topically (for example, as a cream, ointment or spray applied to the skin). The compound may also be formulated for inhalation. In certain embodiments, a compound may be simply dissolved or suspended in sterile water. Details of appropriate routes of administration and compositions suitable for same can be found in, for example, U.S. Pat. Nos. 6,110,973, 5,763,493, 5,731,000, 5,541,231, 5,427,798, 5,358,970 and 4,172,896, as well as in patents cited therein.

The formulations may conveniently be presented in unit dosage form and may be prepared by any methods well known in the art of pharmacy. The amount of active ingredient which can be combined with a carrier material to produce a single dosage form will vary depending upon the host being treated, the particular mode of administration. The amount of active ingredient that can be combined with a carrier material to produce a single dosage form will generally be that amount of the compound which produces a therapeutic effect. Generally, out of one hundred percent, this amount will range from about 1 percent to about ninety-nine percent of active ingredient, preferably from about 5 percent to about 70 percent, most preferably from about 10 percent to about 30 percent.

Methods of preparing these formulations or compositions include the step of bringing into association an active compound, such as a compound of the disclosure, with the carrier and, optionally, one or more accessory ingredients. In general, the formulations are prepared by uniformly and intimately bringing into association a compound of the present disclosure with liquid carriers, or finely divided solid carriers, or both, and then, if necessary, shaping the product.

Formulations of the disclosure suitable for oral administration may be in the form of capsules (including sprinkle capsules and gelatin capsules), cachets, pills, tablets, lozenges (using a flavored basis, usually sucrose and acacia or tragacanth), lyophile, powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of a compound of the present disclosure as an active ingredient. Compositions or compounds may also be administered as a bolus, electuary or paste.

To prepare solid dosage forms for oral administration (capsules (including sprinkle capsules and gelatin capsules), tablets, pills, dragees, powders, granules and the like), the active ingredient is mixed with one or more pharmaceutically acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds; (7) wetting agents, such as, for example, cetyl alcohol and glycerol monostearate; (8) absorbents, such as kaolin and bentonite clay; (9) lubricants, such a talc, calcium stearate, magnesium stearate, solid polyethylene glycols, sodium lauryl sulfate, and mixtures thereof; (10) complexing agents, such as, modified and unmodified cyclodextrins; and (11) coloring agents. In the case of capsules (including sprinkle capsules and gelatin capsules), tablets and pills, the pharmaceutical compositions may also comprise buffering agents. Solid compositions of a similar type may also be employed as fillers in soft and hard-filled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the powdered compound moistened with an inert liquid diluent.

The tablets, and other solid dosage forms of the pharmaceutical compositions, such as dragees, capsules (including sprinkle capsules and gelatin capsules), pills and granules, may optionally be scored or prepared with coatings and shells, such as enteric coatings and other coatings well known in the pharmaceutical-formulating art. They may also be formulated so as to provide slow or controlled release of the active ingredient therein using, for example, hydroxypropylmethyl cellulose in varying proportions to provide the desired release profile, other polymer matrices, liposomes and/or microspheres. They may be sterilized by, for example, filtration through a bacteria-retaining filter, or by incorporating sterilizing agents in the form of sterile solid compositions that can be dissolved in sterile water, or some other sterile injectable medium immediately before use. These compositions may also optionally contain opacifying agents and may be of a composition that they release the active ingredient(s) only, or preferentially, in a certain portion of the gastrointestinal tract, optionally, in a delayed manner. Examples of embedding compositions that can be used include polymeric substances and waxes. The active ingredient can also be in micro-encapsulated form, if appropriate, with one or more of the above-described excipients.

Liquid dosage forms useful for oral administration include pharmaceutically acceptable emulsions, lyophiles for reconstitution, microemulsions, solutions, suspensions, syrups and elixirs. In addition to the active ingredient, the liquid dosage forms may contain inert diluents commonly used in the art, such as, for example, water or other solvents, cyclodextrins and derivatives thereof, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

Besides inert diluents, the oral compositions can also include adjuvants such as wetting agents, emulsifying and suspending agents, sweetening, flavoring, coloring, perfuming and preservative agents.

Suspensions, in addition to the active compounds, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Dosage forms for the topical or transdermal administration include powders, sprays, ointments, pastes, creams, lotions, gels, solutions, patches and inhalants. The active compound may be mixed under sterile conditions with a pharmaceutically acceptable carrier, and with any preservatives, buffers, or propellants that may be required.

The ointments, pastes, creams and gels may contain, in addition to an active compound, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to an active compound, excipients such as lactose, talc, silicic acid, aluminum hydroxide, calcium silicates and polyamide powder, or mixtures of these substances. Sprays can additionally contain customary propellants, such as chlorofluorohydrocarbons and volatile unsubstituted hydrocarbons, such as butane and propane.

Transdermal patches have the added advantage of providing controlled delivery of a compound of the present disclosure to the body. Such dosage forms can be made by dissolving or dispersing the active compound in the proper medium. Absorption enhancers can also be used to increase the flux of the compound across the skin. The rate of such flux can be controlled by either providing a rate controlling membrane or dispersing the compound in a polymer matrix or gel.

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal and intrasternal injection and infusion. Pharmaceutical compositions suitable for parenteral administration comprise one or more active compounds in combination with one or more pharmaceutically acceptable sterile isotonic aqueous or nonaqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents.

Examples of suitable aqueous and nonaqueous carriers that may be employed in the pharmaceutical compositions of the disclosure include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of the action of microorganisms may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents that delay absorption such as aluminum monostearate and gelatin.

In some cases, in order to prolong the effect of a drug, it is desirable to slow the absorption of the drug from subcutaneous or intramuscular injection. This may be accomplished by the use of a liquid suspension of crystalline or amorphous material having poor water solubility. The rate of absorption of the drug then depends upon its rate of dissolution, which, in turn, may depend upon crystal size and crystalline form. Alternatively, delayed absorption of a parenterally administered drug form is accomplished by dissolving or suspending the drug in an oil vehicle.

Injectable depot forms are made by forming microencapsulated matrices of the subject compounds in biodegradable polymers such as polylactide-polyglycolide. Depending on the ratio of drug to polymer, and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions that are compatible with body tissue.

For use in the methods of this disclosure, active compounds can be given per se or as a pharmaceutical composition containing, for example, 0.1 to 99.5% (more preferably, 0.5 to 90%) of active ingredient in combination with a pharmaceutically acceptable carrier.

Methods of introduction may also be provided by rechargeable or biodegradable devices. Various slow release polymeric devices have been developed and tested in vivo in recent years for the controlled delivery of drugs, including proteinaceous biopharmaceuticals. A variety of biocompatible polymers (including hydrogels), including both biodegradable and non-degradable polymers, can be used to form an implant for the sustained release of a compound at a particular target site.

Actual dosage levels of the active ingredients in the pharmaceutical compositions may be varied so as to obtain an amount of the active ingredient that is effective to achieve the desired therapeutic response for a particular patient, composition, and mode of administration, without being toxic to the patient.

The selected dosage level will depend upon a variety of factors including the activity of the particular compound or combination of compounds employed, or the ester, salt or amide thereof, the route of administration, the time of administration, the rate of excretion of the particular compound(s) being employed, the duration of the treatment, other drugs, compounds and/or materials used in combination with the particular compound(s) employed, the age, sex, weight, condition, general health and prior medical history of the patient being treated, and like factors well known in the medical arts.

A physician or veterinarian having ordinary skill in the art can readily determine and prescribe the therapeutically effective amount of the pharmaceutical composition required. For example, the physician or veterinarian could start doses of the pharmaceutical composition or compound at levels lower than that required in order to achieve the desired therapeutic effect and gradually increase the dosage until the desired effect is achieved. By “therapeutically effective amount” is meant the concentration of a compound that is sufficient to elicit the desired therapeutic effect. It is generally understood that the effective amount of the compound will vary according to the weight, sex, age, and medical history of the subject. Other factors which influence the effective amount may include, but are not limited to, the severity of the patient's condition, the disorder being treated, the stability of the compound, and, if desired, another type of therapeutic agent being administered with a compound of the disclosure. A larger total dose can be delivered by multiple administrations of the agent. Methods to determine efficacy and dosage are known to those skilled in the art (Isselbacher et al. (1996) Harrison's Principles of Internal Medicine 13 ed., 1814-1882, herein incorporated by reference).

In general, a suitable daily dose of an active compound used in the compositions and methods of the disclosure will be that amount of the compound that is the lowest dose effective to produce a therapeutic effect. Such an effective dose will generally depend upon the factors described above.

If desired, the effective daily dose of the active compound may be administered as one, two, three, four, five, six or more sub-doses administered separately at appropriate intervals throughout the day, optionally, in unit dosage forms. In certain embodiments of the present disclosure, the active compound may be administered two or three times daily. In preferred embodiments, the active compound will be administered once daily.

The patient receiving this treatment is any animal in need, including primates, in particular humans; and other mammals such as equines, cattle, swine, sheep, cats, and dogs; poultry; and pets in general.

In certain embodiments, compounds of the disclosure may be used alone or conjointly administered with another type of therapeutic agent.

The present disclosure includes the use of pharmaceutically acceptable salts of compounds of the disclosure in the compositions and methods of the present disclosure. In certain embodiments, contemplated salts of the disclosure include, but are not limited to, alkyl, dialkyl, trialkyl or tetra-alkyl ammonium salts. In certain embodiments, contemplated salts of the disclosure include, but are not limited to, L-arginine, benenthamine, benzathine, betaine, calcium hydroxide, choline, deanol, diethanolamine, diethylamine, 2-(diethylamino)ethanol, ethanolamine, ethylenediamine, N-methylglucamine, hydrabamine, 1H-imidazole, lithium, L-lysine, magnesium, 4-(2-hydroxyethyl)morpholine, piperazine, potassium, 1-(2-hydroxyethyl)pyrrolidine, sodium, triethanolamine, tromethamine, and zinc salts. In certain embodiments, contemplated salts of the disclosure include, but are not limited to, Na, Ca, K, Mg, Zn or other metal salts. In certain embodiments, contemplated salts of the disclosure include, but are not limited to, 1-hydroxy-2-naphthoic acid, 2,2-dichloroacetic acid, 2-hydroxyethanesulfonic acid, 2-oxoglutaric acid, 4-acetamidobenzoic acid, 4-aminosalicylic acid, acetic acid, adipic acid, 1-ascorbic acid, 1-aspartic acid, benzenesulfonic acid, benzoic acid, (+)-camphoric acid, (+)-camphor-10-sulfonic acid, capric acid (decanoic acid), caproic acid (hexanoic acid), caprylic acid (octanoic acid), carbonic acid, cinnamic acid, citric acid, cyclamic acid, dodecylsulfuric acid, ethane-1,2-disulfonic acid, ethanesulfonic acid, formic acid, fumaric acid, galactaric acid, gentisic acid, d-glucoheptonic acid, d-gluconic acid, d-glucuronic acid, glutamic acid, glutaric acid, glycerophosphoric acid, glycolic acid, hippuric acid, hydrobromic acid, hydrochloric acid, isobutyric acid, lactic acid, lactobionic acid, lauric acid, maleic acid, 1-malic acid, malonic acid, mandelic acid, methanesulfonic acid , naphthalene-1,5-disulfonic acid, naphthalene-2-sulfonic acid, nicotinic acid, nitric acid, oleic acid, oxalic acid, palmitic acid, pamoic acid, phosphoric acid, proprionic acid, 1-pyroglutamic acid, salicylic acid, sebacic acid, stearic acid, succinic acid, sulfuric acid, 1-tartaric acid, thiocyanic acid, p-toluenesulfonic acid, trifluoroacetic acid, and undecylenic acid acid salts.

The pharmaceutically acceptable acid addition salts can also exist as various solvates, such as with water, methanol, ethanol, dimethylformamide, and the like. Mixtures of such solvates can also be prepared. The source of such solvate can be from the solvent of crystallization, inherent in the solvent of preparation or crystallization, or adventitious to such solvent.

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Examples of pharmaceutically acceptable antioxidants include: (1) water-soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal-chelating agents, such as citric acid, ethylenediamine tetraacetic acid (EDTA), sorbitol, tartaric acid, phosphoric acid, and the like.

### Methods of Treatment

In certain aspects, the present disclosure provides methods of treating a disease or condition, such as a metabolic disease or a cardiovascular disease, in an individual in need thereof, comprising administering a therapeutically effective amount of the compounds provided herein. In certain aspects, the disease or condition is non-alcoholic fatty liver disease (NAFLD), obesity, atherosclerosis, atherosclerotic heart disease, stroke, dyslipidemia, or type 2 diabetes. While not being bound by theory, it is believed that the compounds disclosed herein act as Them1 inhibitors.

### Definitions

Unless otherwise defined herein, scientific and technical terms used in this application shall have the meanings that are commonly understood by those of ordinary skill in the art. Generally, nomenclature used in connection with, and techniques of, chemistry, cell and tissue culture, molecular biology, cell and cancer biology, neurobiology, neurochemistry, virology, immunology, microbiology, pharmacology, genetics and protein and nucleic acid chemistry, described herein, are those well known and commonly used in the art.

The methods and techniques of the present disclosure are generally performed, unless otherwise indicated, according to conventional methods well known in the art and as described in various general and more specific references that are cited and discussed throughout this specification. See, e.g. “Principles of Neural Science”, McGraw-Hill Medical, New York, N.Y. (2000); Motulsky, “Intuitive Biostatistics”, Oxford University Press, Inc. (1995); Lodish et al., “Molecular Cell Biology, 4th ed.”, W. H. Freeman & Co., New York (2000); Griffiths et al., “Introduction to Genetic Analysis, 7th ed.”, W. H. Freeman & Co., N.Y. (1999); and Gilbert et al., “Developmental Biology, 6th ed.”, Sinauer Associates, Inc., Sunderland, MA (2000).

Chemistry terms used herein, unless otherwise defined herein, are used according to conventional usage in the art, as exemplified by “The McGraw-Hill Dictionary of Chemical Terms”, Parker S., Ed., McGraw-Hill, San Francisco, C.A. (1985).

All of the above, and any other publications, patents and published patent applications referred to in this application are specifically incorporated by reference herein. In case of conflict, the present specification, including its specific definitions, will control.

The term “agent” is used herein to denote a chemical compound (such as an organic or inorganic compound, a mixture of chemical compounds), a biological macromolecule (such as a nucleic acid, an antibody, including parts thereof as well as humanized, chimeric and human antibodies and monoclonal antibodies, a protein or portion thereof, e.g., a peptide, a lipid, a carbohydrate), or an extract made from biological materials such as bacteria, plants, fungi, or animal (particularly mammalian) cells or tissues. Agents include, for example, agents whose structure is known, and those whose structure is not known. The ability of such agents to inhibit AR or promote AR degradation may render them suitable as “therapeutic agents” in the methods and compositions of this disclosure.

A “patient,” “subject,” or “individual” are used interchangeably and refer to either a human or a non-human animal. These terms include mammals, such as humans, primates, livestock animals (including bovines, porcines, etc.), companion animals (e.g., canines, felines, etc.) and rodents (e.g., mice and rats).

“Treating” a condition or patient refers to taking steps to obtain beneficial or desired results, including clinical results. As used herein, and as well understood in the art, “treatment” is an approach for obtaining beneficial or desired results, including clinical results. Beneficial or desired clinical results can include, but are not limited to, alleviation or amelioration of one or more symptoms or conditions, diminishment of extent of disease, stabilized (i.e. not worsening) state of disease, preventing spread of disease, delay or slowing of disease progression, amelioration or palliation of the disease state, and remission (whether partial or total), whether detectable or undetectable. “Treatment” can also mean prolonging survival as compared to expected survival if not receiving treatment.

The term “preventing” is art-recognized, and when used in relation to a condition, such as a local recurrence (e.g., pain), a disease such as cancer, a syndrome complex such as heart failure or any other medical condition, is well understood in the art, and includes administration of a composition which reduces the frequency of, or delays the onset of, symptoms of a medical condition in a subject relative to a subject which does not receive the composition. Thus, prevention of cancer includes, for example, reducing the number of detectable cancerous growths in a population of patients receiving a prophylactic treatment relative to an untreated control population, and/or delaying the appearance of detectable cancerous growths in a treated population versus an untreated control population, e.g., by a statistically and/or clinically significant amount.

“Administering” or “administration of” a substance, a compound or an agent to a subject can be carried out using one of a variety of methods known to those skilled in the art. For example, a compound or an agent can be administered, intravenously, arterially, intradermally, intramuscularly, intraperitoneally, subcutaneously, ocularly, sublingually, orally (by ingestion), intranasally (by inhalation), intraspinally, intracerebrally, and transdermally (by absorption, e.g., through a skin duct). A compound or agent can also appropriately be introduced by rechargeable or biodegradable polymeric devices or other devices, e.g., patches and pumps, or formulations, which provide for the extended, slow or controlled release of the compound or agent. Administering can also be performed, for example, once, a plurality of times, and/or over one or more extended periods.

Appropriate methods of administering a substance, a compound or an agent to a subject will also depend, for example, on the age and/or the physical condition of the subject and the chemical and biological properties of the compound or agent (e.g., solubility, digestibility, bioavailability, stability and toxicity). In some embodiments, a compound or an agent is administered orally, e.g., to a subject by ingestion. In some embodiments, the orally administered compound or agent is in an extended release or slow release formulation, or administered using a device for such slow or extended release.

As used herein, the phrase “conjoint administration” refers to any form of administration of two or more different therapeutic agents such that the second agent is administered while the previously administered therapeutic agent is still effective in the body (e.g., the two agents are simultaneously effective in the patient, which may include synergistic effects of the two agents). For example, the different therapeutic compounds can be administered either in the same formulation or in separate formulations, either concomitantly or sequentially. Thus, an individual who receives such treatment can benefit from a combined effect of different therapeutic agents.

A “therapeutically effective amount” or a “therapeutically effective dose” of a drug or agent is an amount of a drug or an agent that, when administered to a subject will have the intended therapeutic effect. The full therapeutic effect does not necessarily occur by administration of one dose, and may occur only after administration of a series of doses. Thus, a therapeutically effective amount may be administered in one or more administrations. The precise effective amount needed for a subject will depend upon, for example, the subject's size, health and age, and the nature and extent of the condition being treated, such as cancer or MDS. The skilled worker can readily determine the effective amount for a given situation by routine experimentation.

As used herein, the terms “optional” or “optionally” mean that the subsequently described event or circumstance may occur or may not occur, and that the description includes instances where the event or circumstance occurs as well as instances in which it does not. For example, “optionally substituted alkyl” refers to the alkyl may be substituted as well as where the alkyl is not substituted.

It is understood that substituents and substitution patterns on the compounds of the present disclosure can be selected by one of ordinary skilled person in the art to result chemically stable compounds which can be readily synthesized by techniques known in the art, as well as those methods set forth below, from readily available starting materials. If a substituent is itself substituted with more than one group, it is understood that these multiple groups may be on the same carbon or on different carbons, so long as a stable structure results.

As used herein, the term “optionally substituted” refers to the replacement of one to six hydrogen radicals in a given structure with the radical of a specified substituent including, but not limited to: hydroxyl, hydroxyalkyl, alkoxy, halogen, alkyl, nitro, silyl, acyl, acyloxy, aryl, cycloalkyl, heterocyclyl, amino, aminoalkyl, cyano, haloalkyl, haloalkoxy, —OCO—CH₂—O-alkyl, —OP(O )(O-alkyl)₂ or —CH₂—OP(O)(O-alkyl)₂. Preferably, “optionally substituted” refers to the replacement of one to four hydrogen radicals in a given structure with the substituents mentioned above. More preferably, one to three hydrogen radicals are replaced by the substituents as mentioned above. It is understood that the substituent can be further substituted.

As used herein, the term “alkyl” refers to saturated aliphatic groups, including but not limited to C₁-C₁₀ straight-chain alkyl groups or C₁-C₁₀ branched-chain alkyl groups. Preferably, the “alkyl” group refers to C₁-C₆ straight-chain alkyl groups or C₁-C₆ branched-chain alkyl groups. Most preferably, the “alkyl” group refers to C₁-C₄ straight-chain alkyl groups or C₁-C₄ branched-chain alkyl groups. Examples of “alkyl” include, but are not limited to, methyl, ethyl, 1-propyl, 2-propyl, n-butyl, sec-butyl, tert-butyl, 1-pentyl, 2-pentyl, 3-pentyl, neo-pentyl, 1-hexyl, 2-hexyl, 3-hexyl, 1-heptyl, 2-heptyl, 3-heptyl, 4-heptyl, 1-octyl, 2-octyl, 3-octyl or 4-octyl and the like. The “alkyl” group may be optionally substituted.

The term “acyl” is art-recognized and refers to a group represented by the general formula hydrocarbylC(O)—, preferably alkylC(O)—.

The term “acylamino” is art-recognized and refers to an amino group substituted with an acyl group and may be represented, for example, by the formula hydrocarbylC(O)NH—.

The term “acyloxy” is art-recognized and refers to a group represented by the general formula hydrocarbylC(O)O—, preferably alkylC(O)O—.

The term “alkoxy” refers to an alkyl group having an oxygen attached thereto. Representative alkoxy groups include methoxy, ethoxy, propoxy, tert-butoxy and the like.

The term “alkoxyalkyl” refers to an alkyl group substituted with an alkoxy group and may be represented by the general formula alkyl-O-alkyl.

The term “alkyl” refers to saturated aliphatic groups, including straight-chain alkyl groups, branched-chain alkyl groups, cycloalkyl (alicyclic) groups, alkyl-substituted cycloalkyl groups, and cycloalkyl-substituted alkyl groups. In preferred embodiments, a straight chain or branched chain alkyl has 30 or fewer carbon atoms in its backbone (e.g., C₁₋₃₀ for straight chains, C₃₋₃₀ for branched chains), and more preferably 20 or fewer.

Moreover, the term “alkyl” as used throughout the specification, examples, and claims is intended to include both unsubstituted and substituted alkyl groups, the latter of which refers to alkyl moieties having substituents replacing a hydrogen on one or more carbons of the hydrocarbon backbone, including haloalkyl groups such as trifluoromethyl and 2,2,2-trifluoroethyl, etc.

The term “C_{x-y}” or “Cₓ-C_{y}”, when used in conjunction with a chemical moiety, such as, acyl, acyloxy, alkyl, alkenyl, alkynyl, or alkoxy is meant to include groups that contain from x to y carbons in the chain. Coalkyl indicates a hydrogen where the group is in a terminal position, a bond if internal. A C₁₋₆alkyl group, for example, contains from one to six carbon atoms in the chain.

The term “alkylamino”, as used herein, refers to an amino group substituted with at least one alkyl group.

The term “alkylthio”, as used herein, refers to a thiol group substituted with an alkyl group and may be represented by the general formula alkylS—.

The term “amide”, as used herein, refers to a group

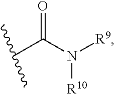

wherein R⁹ and R¹⁰ each independently represent a hydrogen or hydrocarbyl group, or R⁹ and R¹⁰ taken together with the N atom to which they are attached complete a heterocycle having from 4 to 8 atoms in the ring structure.

The terms “amine” and “amino” are art-recognized and refer to both unsubstituted and substituted amines and salts thereof, e.g., a moiety that can be represented by

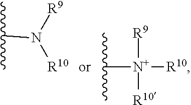

wherein R⁹, R¹⁰, and R^{10′} each independently represent a hydrogen or a hydrocarbyl group, or R⁹ and R¹⁰ taken together with the N atom to which they are attached complete a heterocycle 10 having from 4 to 8 atoms in the ring structure.

The term “aminoalkyl”, as used herein, refers to an alkyl group substituted with an amino group.

The term “aralkyl”, as used herein, refers to an alkyl group substituted with an aryl group.

The term “aryl” as used herein include substituted or unsubstituted single-ring aromatic groups in which each atom of the ring is carbon. Preferably the ring is a 5- to 7-membered ring, more preferably a 6-membered ring. The term “aryl” also includes polycyclic ring systems having two or more cyclic rings in which two or more carbons are common to two adjoining rings wherein at least one of the rings is aromatic, e.g., the other cyclic rings can be cycloalkyls, cycloalkenyls, cycloalkynyls, aryls, heteroaryls, and/or heterocyclyls. Aryl groups include benzene, naphthalene, phenanthrene, phenol, aniline, and the like.

The term “carbamate” is art-recognized and refers to a group

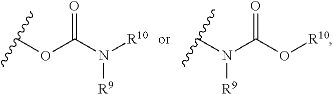

wherein R⁹ and R¹⁰ independently represent hydrogen or a hydrocarbyl group.

The term “carbocyclylalkyl”, as used herein, refers to an alkyl group substituted with a carbocycle group.

The term “carbocycle” includes 5-7 membered monocyclic and 8-12 membered bicyclic rings. Each ring of a bicyclic carbocycle may be selected from saturated, unsaturated and aromatic rings. Carbocycle includes bicyclic molecules in which one, two or three or more atoms are shared between the two rings. The term “fused carbocycle” refers to a bicyclic carbocycle in which each of the rings shares two adjacent atoms with the other ring. Each ring of a fused carbocycle may be selected from saturated, unsaturated and aromatic rings. In an exemplary embodiment, an aromatic ring, e.g., phenyl, may be fused to a saturated or unsaturated ring, e.g., cyclohexane, cyclopentane, or cyclohexene. Any combination of saturated, unsaturated and aromatic bicyclic rings, as valence permits, is included in the definition of carbocyclic. Exemplary “carbocycles” include cyclopentane, cyclohexane, bicyclo[2.2.1]heptane, 1,5-cyclooctadiene, 1,2,3,4-tetrahydronaphthalene, bicyclo[4.2.0]oct-3-ene, naphthalene and adamantane. Exemplary fused carbocycles include decalin, naphthalene, 1,2,3,4-tetrahydronaphthalene, bicyclo[4.2.0]octane, 4,5,6,7-tetrahydro-1H-indene and bicyclo[4.1.0]hept-3-ene. “Carbocycles” may be substituted at any one or more positions capable of bearing a hydrogen atom.

term “carbocyclylalkyl”, as used herein, refers to an alkyl group substituted with a carbocycle group.

The term “carbonate” is art-recognized and refers to a group —OCO₂—.

The term “carboxy”, as used herein, refers to a group represented by the formula —CO₂H.

The term “ester”, as used herein, refers to a group —C(O)OR⁹ wherein R⁹ represents a hydrocarbyl group.

The term “ether”, as used herein, refers to a hydrocarbyl group linked through an oxygen to another hydrocarbyl group. Accordingly, an ether substituent of a hydrocarbyl group may be hydrocarbyl-O—. Ethers may be either symmetrical or unsymmetrical. Examples of ethers include, but are not limited to, heterocycle-O-heterocycle and aryl-O-heterocycle. Ethers include “alkoxyalkyl” groups, which may be represented by the general formula alkyl-O-alkyl.

The terms “halo” and “halogen” as used herein means halogen and includes chloro, fluoro, bromo, and iodo.

The terms “hetaralkyl” and “heteroaralkyl”, as used herein, refers to an alkyl group substituted with a hetaryl group.

The terms “heteroaryl” and “hetaryl” include substituted or unsubstituted aromatic single ring structures, preferably 5- to 7-membered rings, more preferably 5- to 6-membered rings, whose ring structures include at least one heteroatom, preferably one to four heteroatoms, more preferably one or two heteroatoms. The terms “heteroaryl” and “hetaryl” also include polycyclic ring systems having two or more cyclic rings in which two or more carbons are common to two adjoining rings wherein at least one of the rings is heteroaromatic, e.g., the other cyclic rings can be cycloalkyls, cycloalkenyls, cycloalkynyls, aryls, heteroaryls, and/or heterocyclyls. Heteroaryl groups include, for example, pyrrole, furan, thiophene, imidazole, oxazole, thiazole, pyrazole, pyridine, pyrazine, pyridazine, and pyrimidine, and the like.

The term “heteroatom” as used herein means an atom of any element other than carbon or hydrogen. Preferred heteroatoms are nitrogen, oxygen, and sulfur.

The term “heterocyclylalkyl”, as used herein, refers to an alkyl group substituted with a heterocycle group.

The terms “heterocyclyl”, “heterocycle”, and “heterocyclic” refer to substituted or unsubstituted non-aromatic ring structures, preferably 3- to 10-membered rings, more preferably 3- to 7-membered rings, whose ring structures include at least one heteroatom, preferably one to four heteroatoms, more preferably one or two heteroatoms. The terms “heterocyclyl” and “heterocyclic” also include polycyclic ring systems having two or more cyclic rings in which two or more carbons are common to two adjoining rings wherein at least one of the rings is heterocyclic, e.g., the other cyclic rings can be cycloalkyls, cycloalkenyls, cycloalkynyls, aryls, heteroaryls, and/or heterocyclyls. Heterocyclyl groups include, for example, piperidine, piperazine, pyrrolidine, morpholine, lactones, lactams, and the like.

The term “hydrocarbyl”, as used herein, refers to a group that is bonded through a carbon atom that does not have a ═O or ═S substituent, and typically has at least one carbon-hydrogen bond and a primarily carbon backbone, but may optionally include heteroatoms. Thus, groups like methyl, ethoxyethyl, 2-pyridyl, and even trifluoromethyl are considered to be hydrocarbyl for the purposes of this application, but substituents such as acetyl (which has a ═O substituent on the linking carbon) and ethoxy (which is linked through oxygen, not carbon) are not. Hydrocarbyl groups include, but are not limited to aryl, heteroaryl, carbocycle, heterocycle, alkyl, alkenyl, alkynyl, and combinations thereof.

The term “hydroxyalkyl”, as used herein, refers to an alkyl group substituted with a hydroxy group.

The term “lower” when used in conjunction with a chemical moiety, such as, acyl, acyloxy, alkyl, alkenyl, alkynyl, or alkoxy is meant to include groups where there are ten or fewer atoms in the substituent, preferably six or fewer. A “lower alkyl”, for example, refers to an alkyl group that contains ten or fewer carbon atoms, preferably six or fewer. In certain embodiments, acyl, acyloxy, alkyl, alkenyl, alkynyl, or alkoxy substituents defined herein are respectively lower acyl, lower acyloxy, lower alkyl, lower alkenyl, lower alkynyl, or lower alkoxy, whether they appear alone or in combination with other substituents, such as in the recitations hydroxyalkyl and aralkyl (in which case, for example, the atoms within the aryl group are not counted when counting the carbon atoms in the alkyl substituent).

The terms “polycyclyl”, “polycycle”, and “polycyclic” refer to two or more rings (e.g., cycloalkyls, cycloalkenyls, cycloalkynyls, aryls, heteroaryls, and/or heterocyclyls) in which two or more atoms are common to two adjoining rings, e.g., the rings are “fused rings”. Each of the rings of the polycycle can be substituted or unsubstituted. In certain embodiments, each ring of the polycycle contains from 3 to 10 atoms in the ring, preferably from 5 to 7.

The term “sulfate” is art-recognized and refers to the group —OSO3H, or a pharmaceutically acceptable salt thereof.

The term “sulfonamide” is art-recognized and refers to the group represented by the general formulae

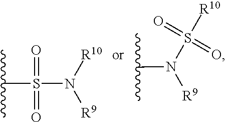

wherein R⁹ and R¹⁰ independently represents hydrogen or hydrocarbyl.

The term “sulfone” is art-recognized and refers to the group represented by the general formulae

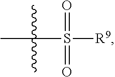

wherein R⁹ represents hydrogen or hydrocarbyl.

The term “sulfoxide” is art-recognized and refers to the group—S(O)—.

The term “sulfonate” is art-recognized and refers to the group SO₃H, or a pharmaceutically acceptable salt thereof.

The term “sulfone” is art-recognized and refers to the group —S(O)₂—.

The term “substituted” refers to moieties having substituents replacing a hydrogen on one or more carbons of the backbone. It will be understood that “substitution” or “substituted with” includes the implicit proviso that such substitution is in accordance with permitted valence of the substituted atom and the substituent, and that the substitution results in a stable compound, e.g., which does not spontaneously undergo transformation such as by rearrangement, cyclization, elimination, etc. As used herein, the term “substituted” is contemplated to include all permissible substituents of organic compounds. In a broad aspect, the permissible substituents include acyclic and cyclic, branched and unbranched, carbocyclic and heterocyclic, aromatic and non-aromatic substituents of organic compounds. The permissible substituents can be one or more and the same or different for appropriate organic compounds. For purposes of this disclosure, the heteroatoms such as nitrogen may have hydrogen substituents and/or any permissible substituents of organic compounds described herein which satisfy the valences of the heteroatoms. Substituents can include any substituents described herein, for example, a halogen, a hydroxyl, a carbonyl (such as a carboxyl, an alkoxycarbonyl, a formyl, or an acyl), a thiocarbonyl (such as a thioester, a thioacetate, or a thioformate), an alkoxyl, a phosphoryl, a phosphate, a phosphonate, a phosphinate, an amino, an amido, an amidine, an imine, a cyano, a nitro, an azido, a sulfhydryl, an alkylthio, a sulfate, a sulfonate, a sulfamoyl, a sulfonamido, a sulfonyl, a heterocyclyl, an aralkyl, or an aromatic or heteroaromatic moiety. It will be understood by those skilled in the art that the moieties substituted on the hydrocarbon chain can themselves be substituted, if appropriate.

The term “thioalkyl”, as used herein, refers to an alkyl group substituted with a thiol group.

The term “thioester”, as used herein, refers to a group —C(O)SR⁹ or —SC(O)R⁹

wherein R⁹ represents a hydrocarbyl.

The term “thioether”, as used herein, is equivalent to an ether, wherein the oxygen is replaced with a sulfur.

The term “urea” is art-recognized and may be represented by the general formula

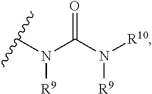

wherein R⁹ and R¹⁰ independently represent hydrogen or a hydrocarbyl.

The term “modulate” as used herein includes the inhibition or suppression of a function or activity (such as cell proliferation) as well as the enhancement of a function or activity.

The phrase “pharmaceutically acceptable” is art-recognized. In certain embodiments, the term includes compositions, excipients, adjuvants, polymers and other materials and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

“Pharmaceutically acceptable salt” or “salt” is used herein to refer to an acid addition salt or a basic addition salt which is suitable for or compatible with the treatment of patients.

The term “pharmaceutically acceptable acid addition salt” as used herein means any non-toxic organic or inorganic salt of any base compounds represented by Formula I. Illustrative inorganic acids which form suitable salts include hydrochloric, hydrobromic, sulfuric and phosphoric acids, as well as metal salts such as sodium monohydrogen orthophosphate and potassium hydrogen sulfate. Illustrative organic acids that form suitable salts include mono-, di-, and tricarboxylic acids such as glycolic, lactic, pyruvic, malonic, succinic, glutaric, fumaric, malic, tartaric, citric, ascorbic, maleic, benzoic, phenylacetic, cinnamic and salicylic acids, as well as sulfonic acids such as p-toluene sulfonic and methanesulfonic acids. Either the mono or di-acid salts can be formed, and such salts may exist in either a hydrated, solvated or substantially anhydrous form. In general, the acid addition salts of compounds of Formula I are more soluble in water and various hydrophilic organic solvents, and generally demonstrate higher melting points in comparison to their free base forms. The selection of the appropriate salt will be known to one skilled in the art. Other non-pharmaceutically acceptable salts, e.g., oxalates, may be used, for example, in the isolation of compounds of Formula I for laboratory use, or for subsequent conversion to a pharmaceutically acceptable acid addition salt.

The term “pharmaceutically acceptable basic addition salt” as used herein means any non-toxic organic or inorganic base addition salt of any acid compounds represented by Formula I or any of their intermediates. Illustrative inorganic bases which form suitable salts include lithium, sodium, potassium, calcium, magnesium, or barium hydroxide. Illustrative organic bases which form suitable salts include aliphatic, alicyclic, or aromatic organic amines such as methylamine, trimethylamine and picoline or ammonia. The selection of the appropriate salt will be known to a person skilled in the art.

Many of the compounds useful in the methods and compositions of this disclosure have at least one stereogenic center in their structure. This stereogenic center may be present in a R or a S configuration, said R and S notation is used in correspondence with the rules described in Pure Appl. Chem. (1976), 45, 11-30. The disclosure contemplates all stereoisomeric forms such as enantiomeric and diastereoisomeric forms of the compounds, salts, prodrugs or mixtures thereof (including all possible mixtures of stereoisomers). See, e.g., WO 01/062726.

Furthermore, certain compounds which contain alkenyl groups may exist as Z (zusammen) or E (entgegen) isomers. In each instance, the disclosure includes both mixture and separate individual isomers.

Some of the compounds may also exist in tautomeric forms. Such forms, although not explicitly indicated in the formulae described herein, are intended to be included within the scope of the present disclosure.

“Prodrug” or “pharmaceutically acceptable prodrug” refers to a compound that is metabolized, for example hydrolyzed or oxidized, in the host after administration to form the compound of the present disclosure (e.g., compounds of formula I). Typical examples of prodrugs include compounds that have biologically labile or cleavable (protecting) groups on a functional moiety of the active compound. Prodrugs include compounds that can be oxidized, reduced, aminated, deaminated, hydroxylated, dehydroxylated, hydrolyzed, dehydrolyzed, alkylated, dealkylated, acylated, deacylated, phosphorylated, or dephosphorylated to produce the active compound. Examples of prodrugs using ester or phosphoramidate as biologically labile or cleavable (protecting) groups are disclosed in U.S. Patents 6,875,751, 7,585,851, and 7,964,580, the disclosures of which are incorporated herein by reference. The prodrugs of this disclosure are metabolized to produce a compound of Formula I. The present disclosure includes within its scope, prodrugs of the compounds described herein. Conventional procedures for the selection and preparation of suitable prodrugs are described, for example, in “Design of Prodrugs” Ed. H. Bundgaard, Elsevier, 1985.

The phrase “pharmaceutically acceptable carrier” as used herein means a pharmaceutically acceptable material, composition or vehicle, such as a liquid or solid filter, diluent, excipient, solvent or encapsulating material useful for formulating a drug for medicinal or therapeutic use.

The term “Log of solubility”, “LogS” or “logS” as used herein is used in the art to quantify the aqueous solubility of a compound. The aqueous solubility of a compound significantly affects its absorption and distribution characteristics. A low solubility often goes along with a poor absorption. LogS value is a unit stripped logarithm (base 10) of the solubility measured in mol/liter.

### Discussion

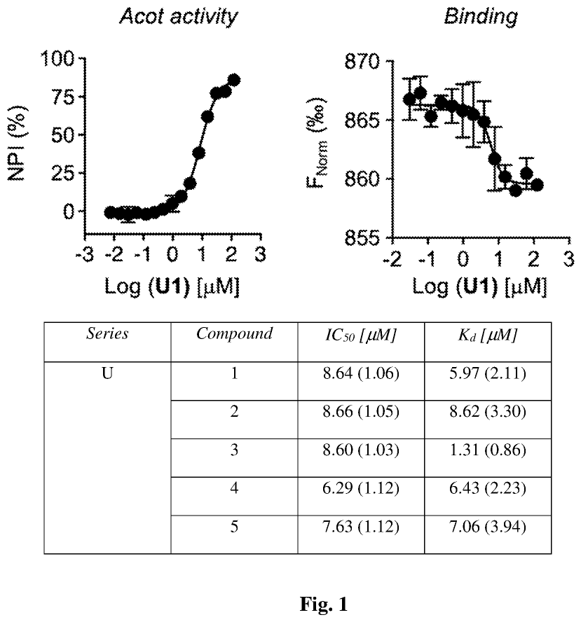

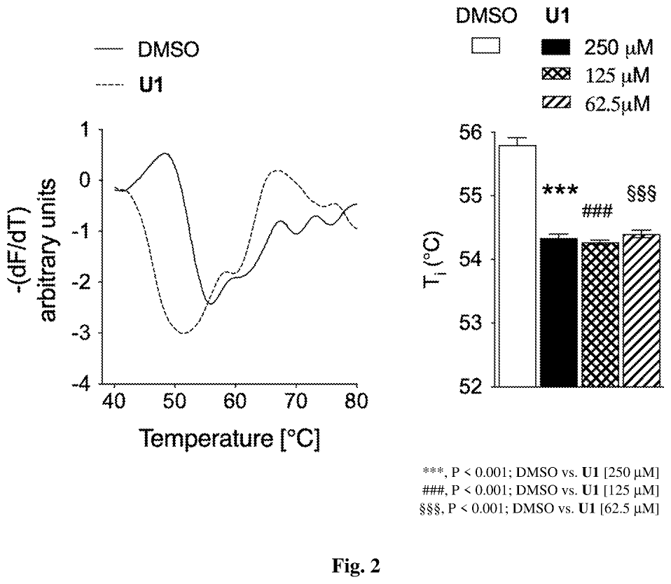

Compound U1 is characterized by a central hydrocarbon decorated with thiophene and piperazine heterocycle rings, and an indole moiety. Structural analogs of compound U1 for which the piperazine heterocycle was replaced improved (U3) or retained comparable (U2, U4 and U5) IC₅₀ and K_{d} values to the parent U1 compound, as described herein. (FIG. 1).

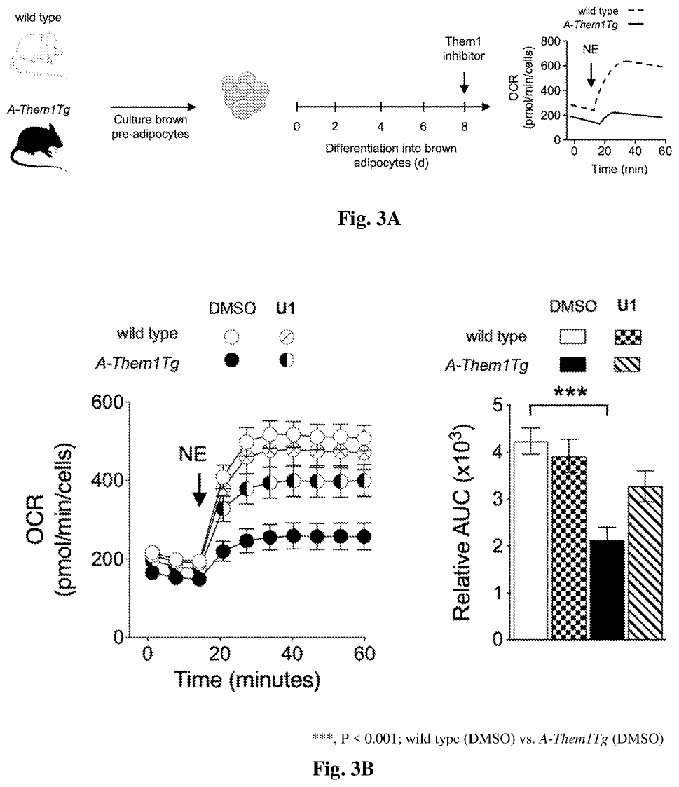

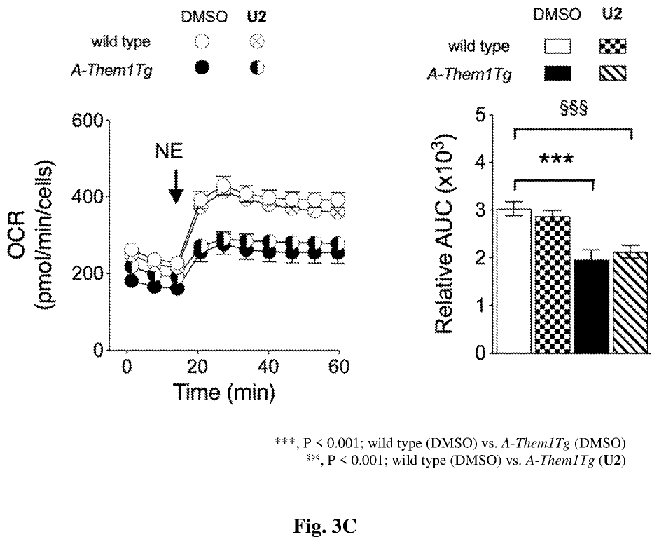

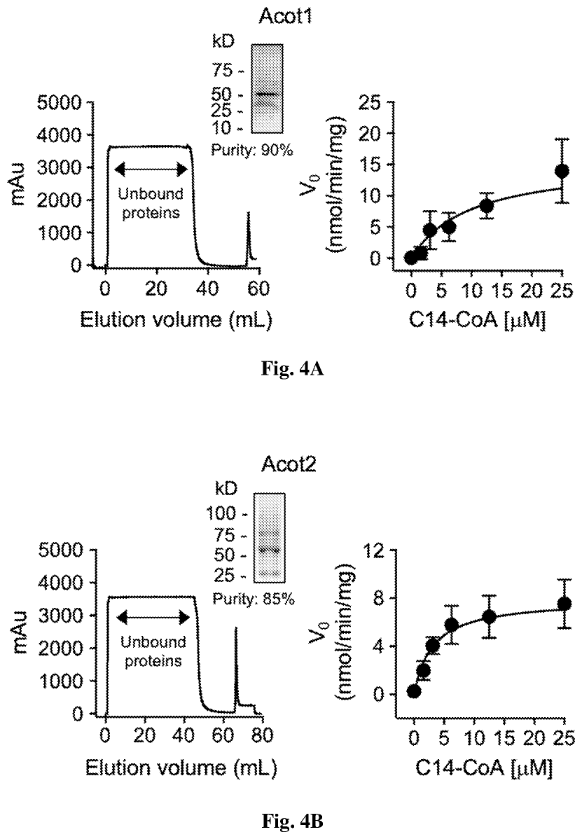

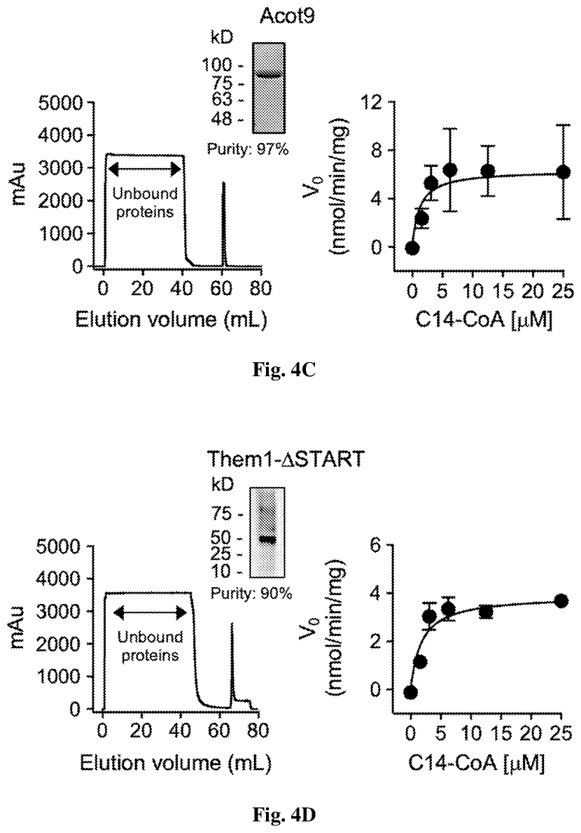

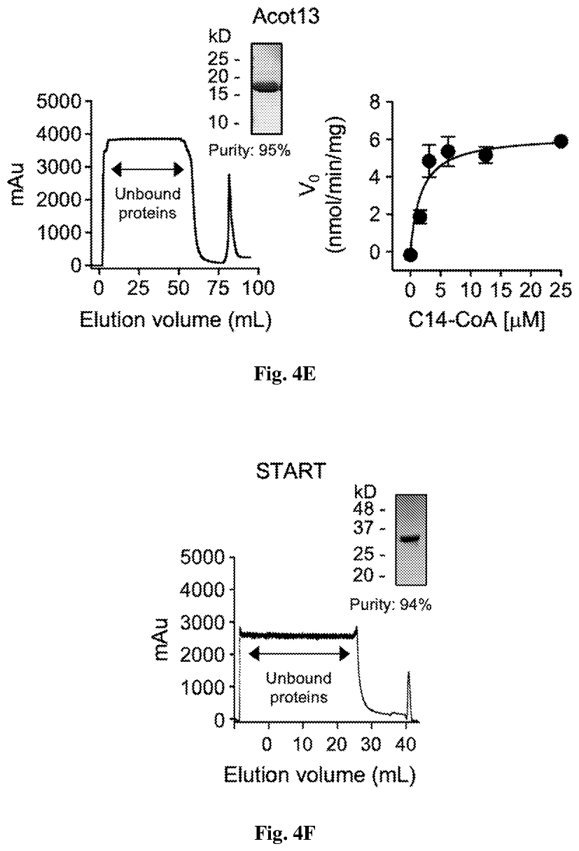

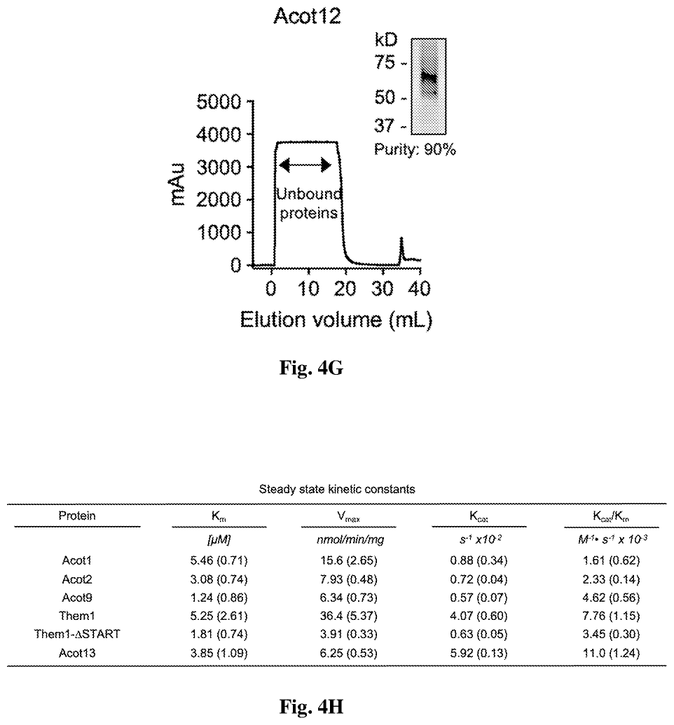

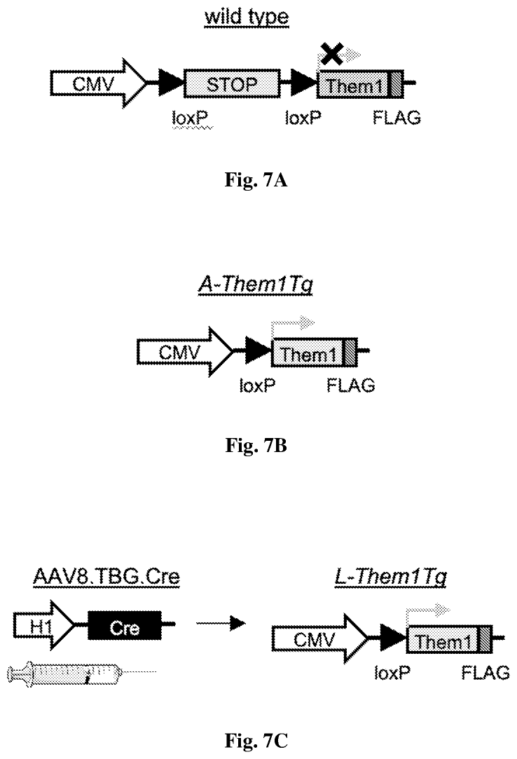

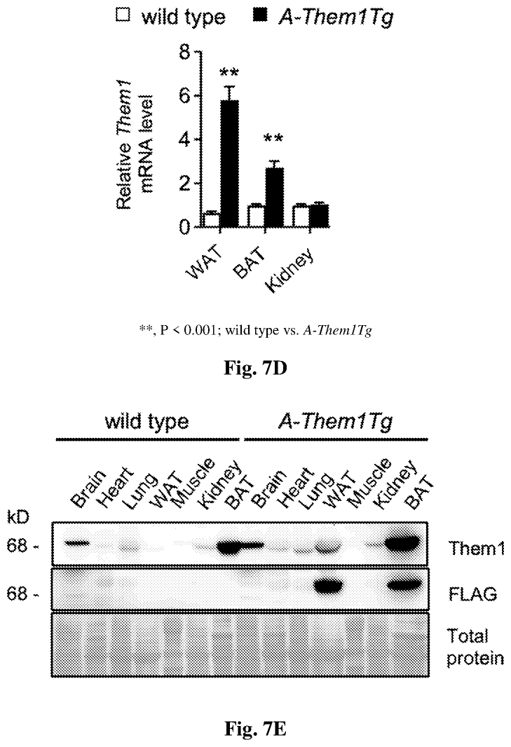

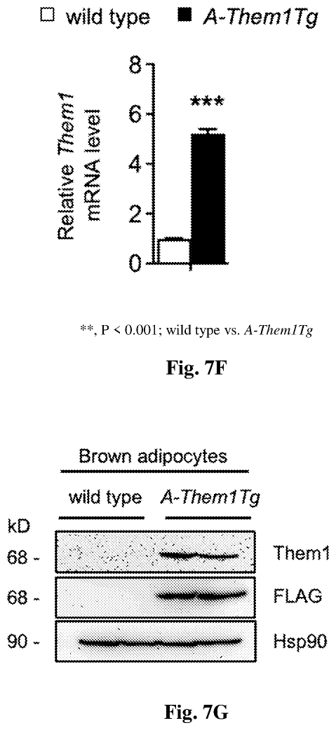

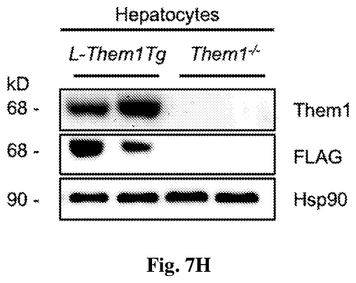

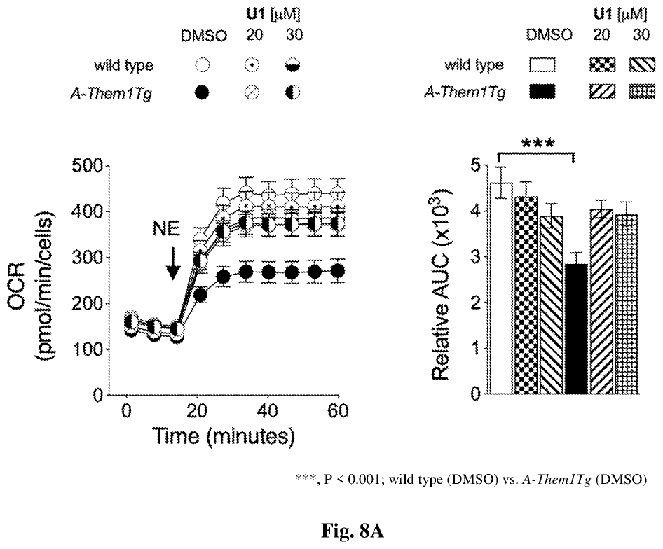

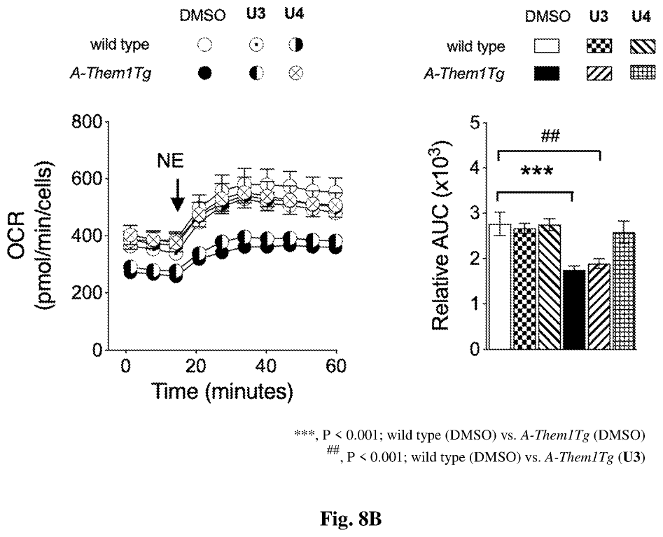

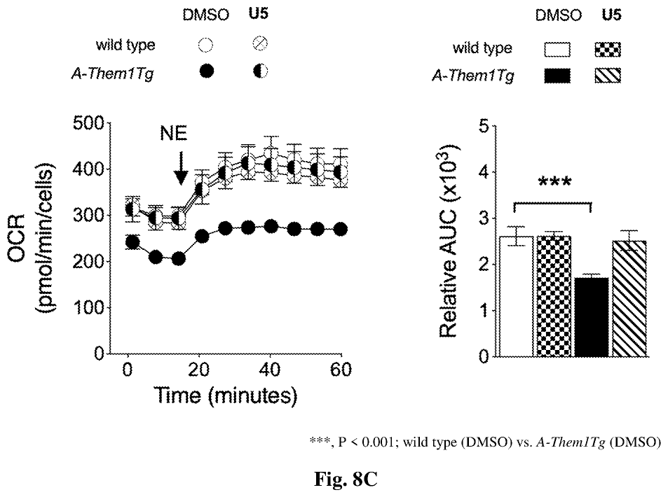

Further development of compounds U1 as potential early lead compounds required a cell culture system with robust expression of active Them1. In this connection, it was previously shown that endogenous Them1 expression was almost completely lost in cultured brown adipocytes, necessitating transduction with recombinant adenovirus in order to achieve expression and to demonstrate Them1-mediated suppression of fatty acid oxidation (Okada et al., 2016; Zhang et al., 2012). By contrast, cultured hepatocytes continued to express Them1, although the level of expression diminished with time. To address these limitations, we generated a transgenic Them1 mouse from which we created mice that overexpress FLAG-Them1 in adipose tissue (A-Them1Tg) or in liver (L-Them1Tg). Mice harbored single copies of the transgene Them1 in the absence (wild type (FIG. 7A) or the presence of a single copy of the transgene adiponectin-Cre (A-Them1Tg) (FIG. 7B) or human thyroxine binding globulin (H1)-Cre (L-Them1Tg) (FIG. 7C). Adipose tissue-specific Them1 transgene overexpression was confirmed in A-Them1Tg mice according to increased Them1 mRNA and protein abundance (FIGS. 7D-E) in WAT and BAT, but not other tissues including brain, heart, lung, skeletal muscle and kidney. FLAG protein abundance was detected exclusively in WAT and BAT of A-Them1Tg mice. In cultured brown adipocytes, Them1 RNA (FIG. 7F) and protein was readily detected in brown adipocytes from A-Them1Tg mice (FIG. 7G). Them1 protein was also robustly expressed in hepatocytes cultured from L-Them1Tg mice (FIG. 7H). A cell-based assay was optimized to measure oxygen consumption rates (FIG. 3A) as a surrogate marker for fatty acid oxidation (Okada et al., 2016). Indicative of Them1 activity, norepinephrine-stimulated OCR values were blunted by Them1 overexpression in A-Them1Tg brown adipocytes reversed the suppressive effects of Them1 on values of OCR compared to wild type brown adipocytes treated with DMSO (FIG. 3B) in a concentration-dependent manner (FIG. 8A). By contrast, treatment of cells with compounds U2-U5, which exhibit similar IC₅₀ and K_{d} values as the parent compound U1, demonstrated similar or reduced capacities to reverse the suppressive effects of Them1 on OCR (FIG. 3C; FIGS. 8B, C).

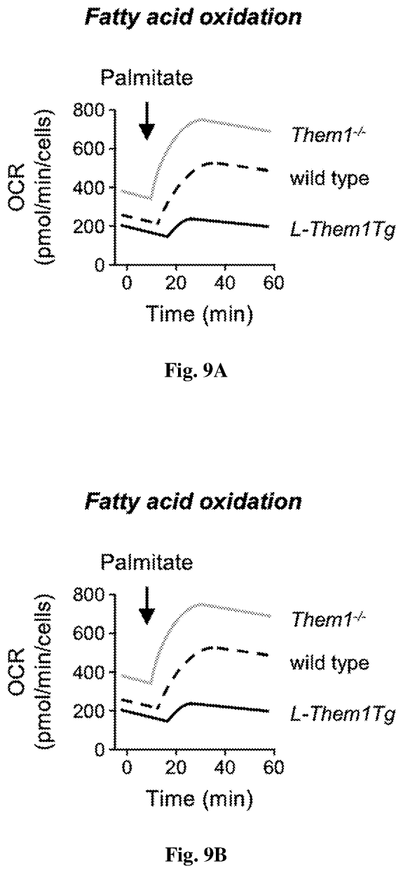

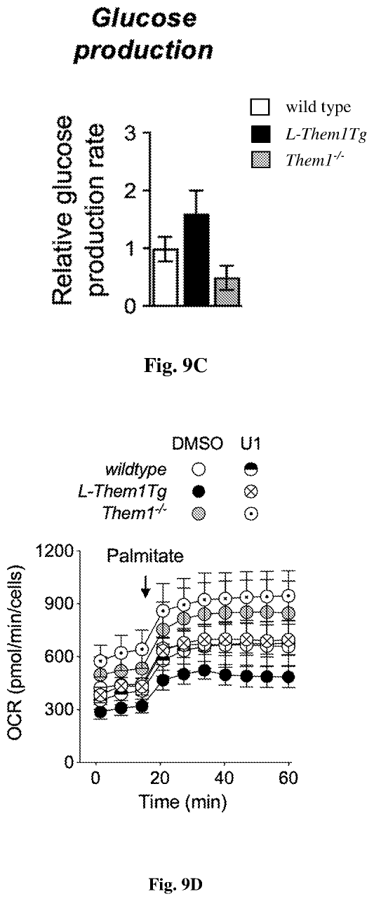

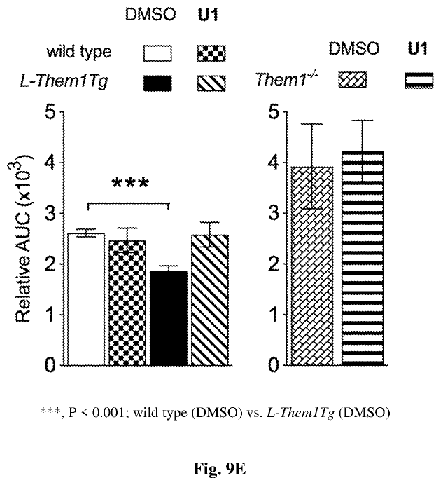

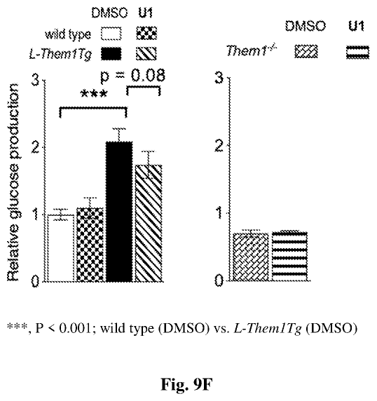

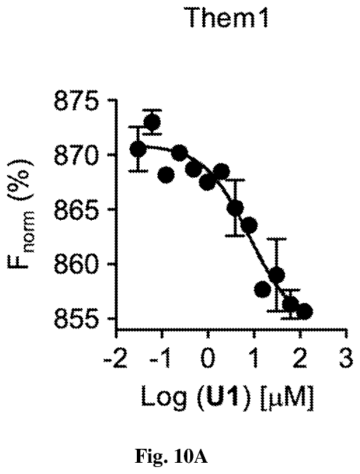

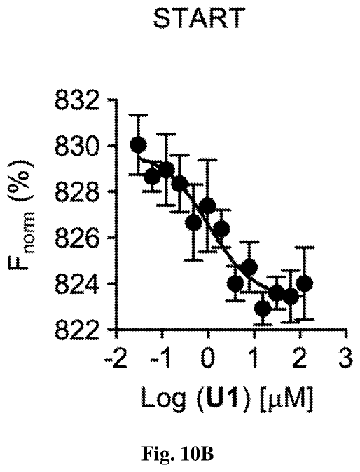

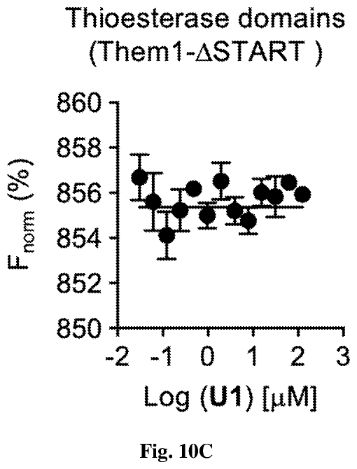

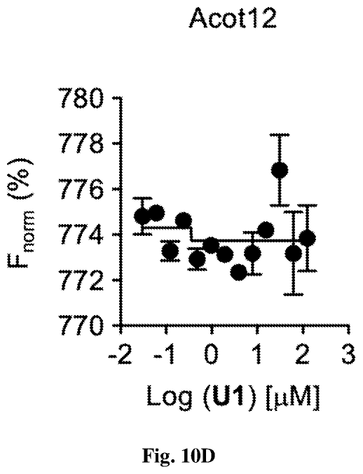

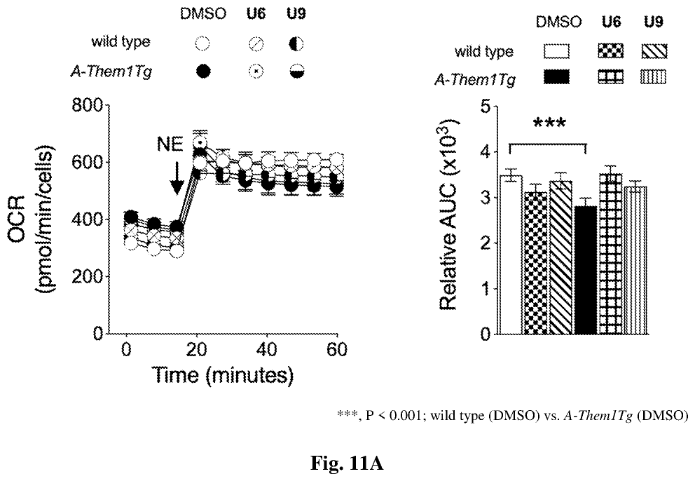

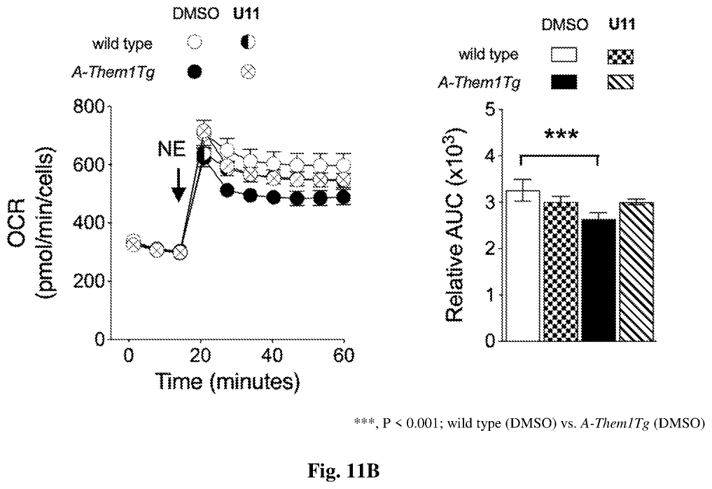

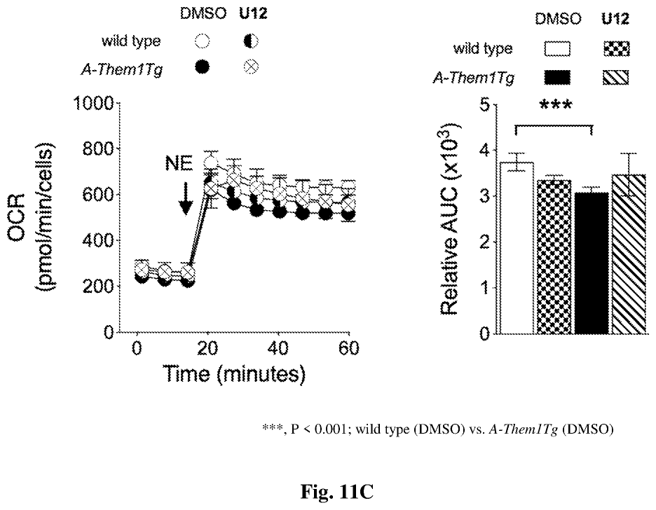

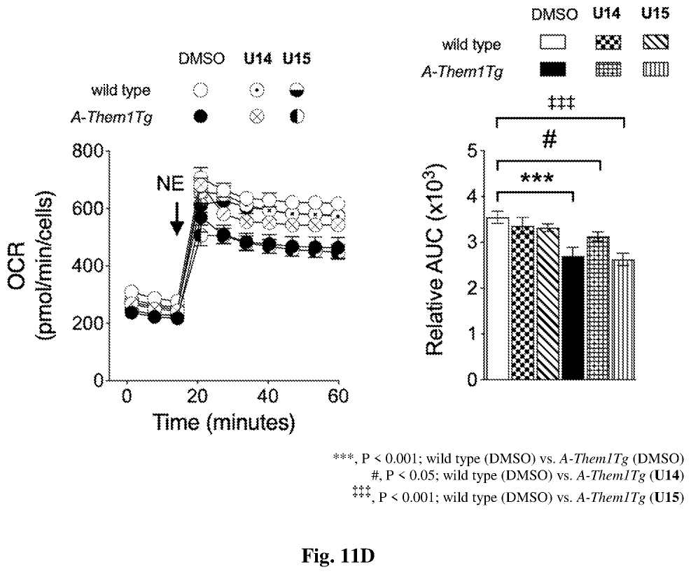

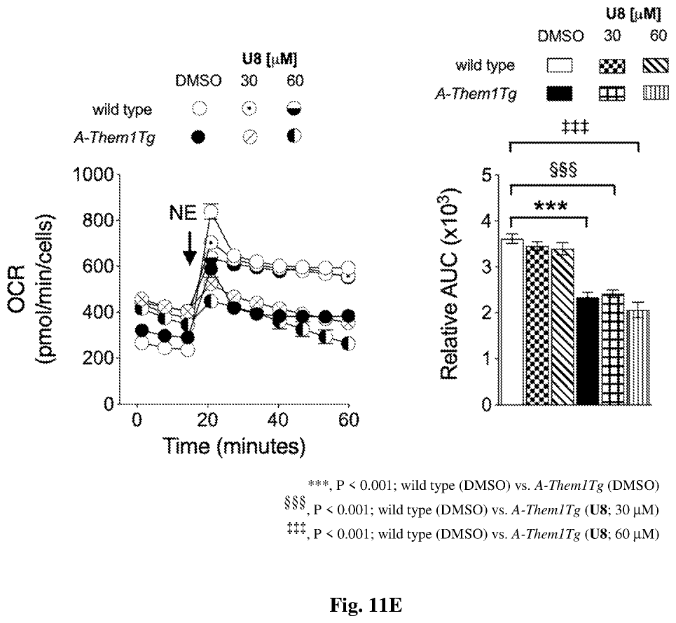

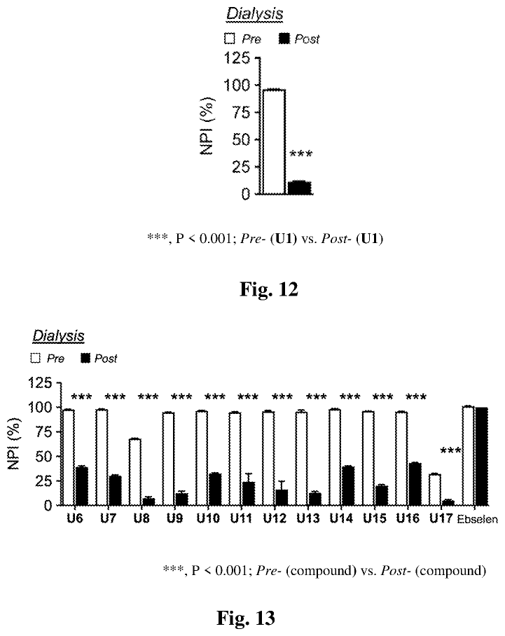

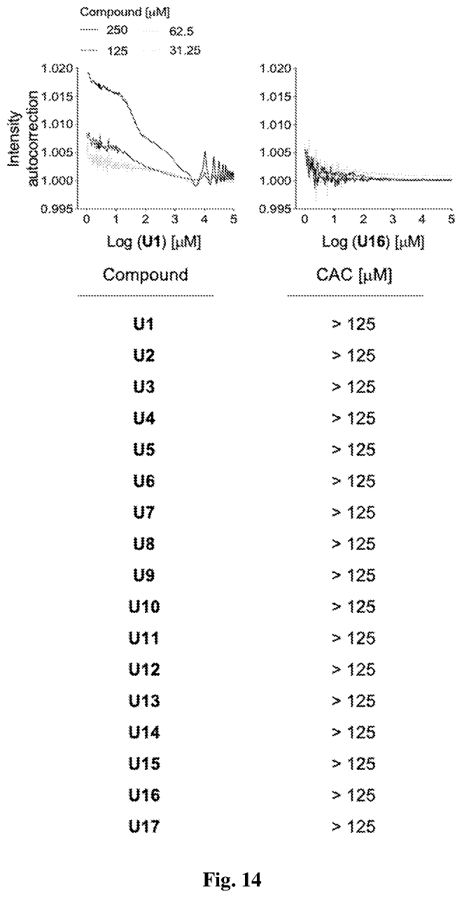

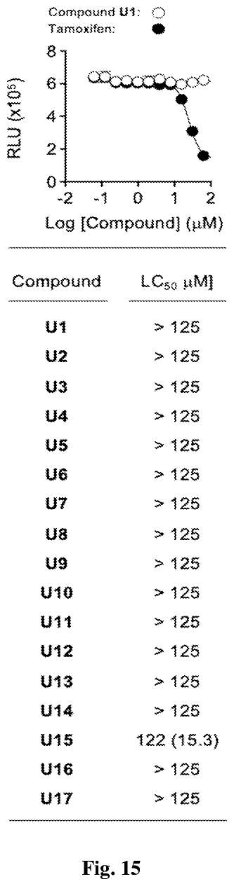

In cultured hepatocytes, cell-based assays were designed to measure OCR as a surrogate marker for fatty acid oxidation of exogenous fatty acids, and glucose production from gluconeogenic substrates (FIGS. 9A-C) (Desai et al., 2018; Kawano et al., 2014). OCR values following palmitate stimulation were reduced in L-Them1Tg hepatocytes. Treatment with compound U1 reversed this suppressive effect of Them1. Consistent with Them1-specific inhibition, compound U1 did not increase OCR values in Them1^{-/-} hepatocytes, which exhibited higher OCR values than wild type or L-Them1Tg hepatocytes (FIGS. 9D-E). Hepatic glucose production was increased by transgenic Them1 overexpression, and this was reduced by treatment with compound U1. Them1 inhibitors had no effect in Them1^{-/-} hepatocytes, which exhibited lower glucose production rates compared with hepatocytes from wild type or L-Them1Tg mice (FIG. 9F).

The binding affinity of compound U1to the START domain (K_{d}: 1.03 μM) was comparable to full-length Them1 (K_{d}: 8.00 μM) and lacked the capacity to bind Them1-ΔSTART.

The helix-grip fold of the Them1 lipid-sensing START domain forms a hydrophobic tunnel that accommodates a lipid molecule (Schrick et al., 2004). Binding of long chain fatty acids allosterically enhances the enzymatic activity of the thioesterase domains (Han and Cohen, 2012; Tillman et al., 2020).

Biological evidence of the therapeutic potential of compound U1 was demonstrated using a cellular-based assay to measure OCR, which serves as a surrogate marker for energy expenditure. Compound U1 reversed the suppressive effects of Them1 activity. Based on this discovery, Compound U1 was selected for more detailed SAR strategies through substitution or extension of critical functional groups within the chemical structure. Values of OCR in primary brown adipocytes treated with structural analogs of compound U1 either remained suppressed or were restored to levels comparable to wild type primary brown adipocytes.

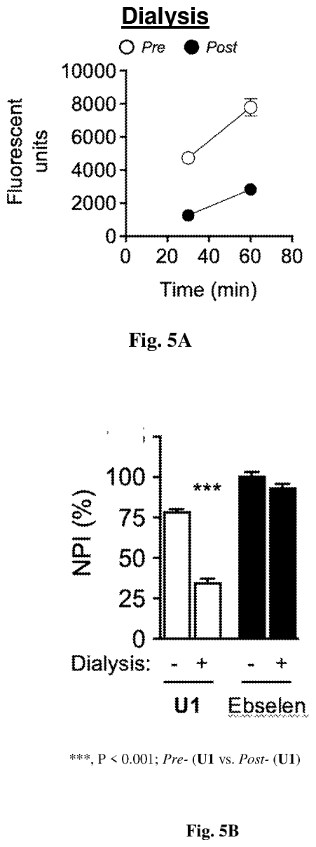

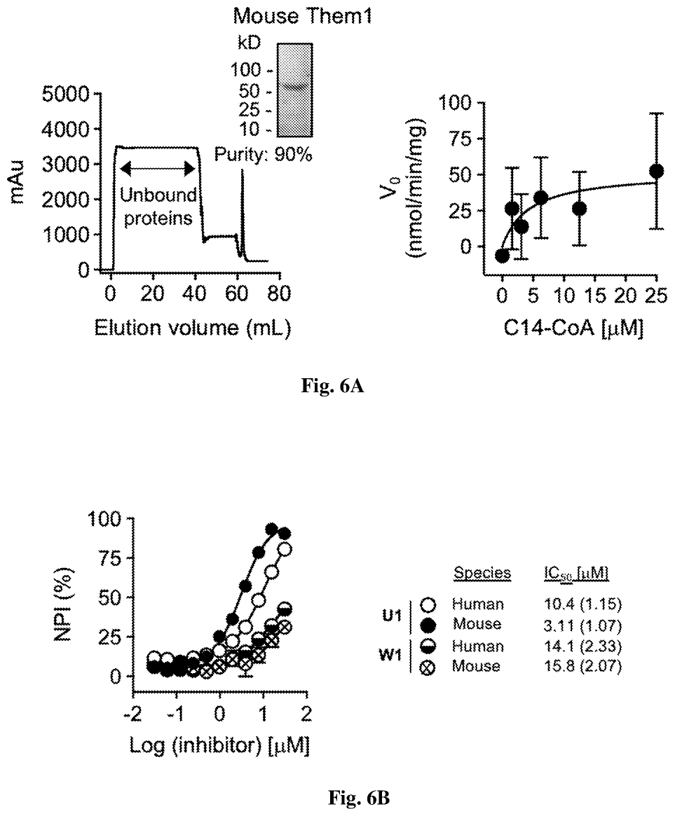

Because it exhibited activity in both primary brown adipocytes and hepatocytes, Compound U1 was selected for more detailed SAR strategies through substitution or expansion of critical functional groups within the chemical structure. Values of OCR in primary brown adipocytes cultured from A-Them1Tg mice treated with structural analogs and derivatives of compound U1 either remained suppressed or were restored to levels comparable to wild type control brown adipocytes. Because these effects were observed within an acute treatment period of 30 min, it is likely that compound U1 and its structural analogs function through reversibly bound protein-inhibitor complexes to inhibit Them1 activity, rather than irreversible covalent inhibition (Singh et al., 2011). To exclude an irreversible mechanism of Them1 acetylation to inhibit enzymatic activity, dialysis was used (FIGS. 5A, B). Dialysis of U1 restored Them1 activity. By contrast, Them1 activity remained suppressed in reactions containing the covalent modifier Ebselen. It was further verified that U1 inhibited the activity of purified recombinant mouse Them1 (FIG. 6A) with comparable potencies to inhibit human Them1 activity (FIG. 6B).

### EXAMPLES

The disclosure now being generally described, it will be more readily understood by reference to the following examples which are included merely for purposes of illustration of certain aspects and embodiments of the present disclosure, and are not intended to limit the disclosure.

### Example 1: Synthesis of Exemplary Compounds of the Disclosure

### Synthesis of Small Molecule Inhibitors. Exemplary Synthetic Procedures and ¹H-NMR and ¹³C-NMR Spectra for the Compound U Series

### Synthesis of Intermediate 1-2

### Step 1

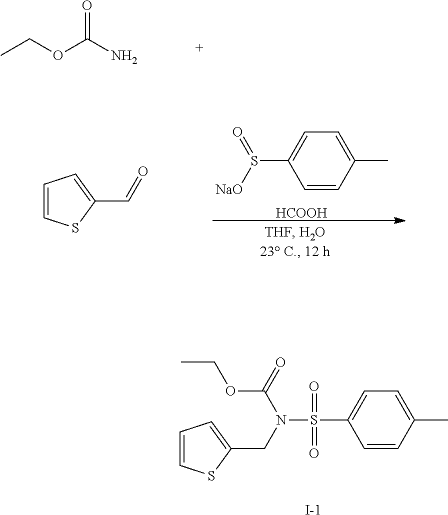

To a solution of ethyl carbamate (500 mg, 5.61 mmol, 1.00 eq) in a mixture of THF (2.00 mL) and H₂O (5.00 mL), was added sodium 4-methylbenzenesulfinate (1.00 g, 5.61 mmol, 1.00 eq) followed by thiophene-2-carbaldehyde (3) (577 μL, 6.17 mmol, 1.10 eq). To this mixture was added formic acid (1.20 mL, 5.61 mmol, 1.00 eq) dropwise at 25° C. The mixture was stirred for 12 h at 25° C. then concentrated under vacuum to obtain ethyl (thiophen-2-ylmethyl)(tosyl)carbamate (I-1) (1.00 g) as a yellow solid. The crude product was used without further purification.

### Step 2

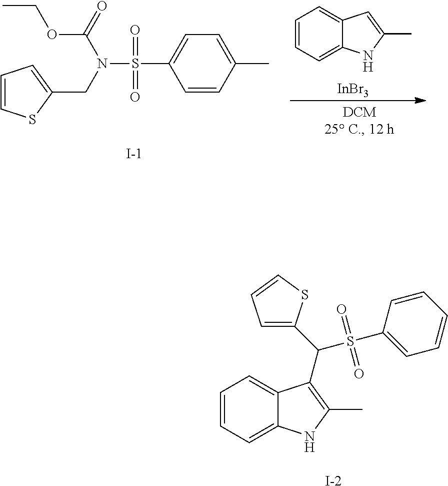

To a solution of compound I-1 (1.00 g, 2.95 mmol, 1.00 eq) and 2-methyl indole (425 mg, 3.24 mmol, 1.10 eq) in DCM (10.0 mL) was added InBr₃ (104 mg, 295 μmol, 0.10 eq) at 25° C. and the mixture was stirred for 12 h. The reaction mixture was diluted with water (20.0 mL) and then extracted with DCM (2×20.0 mL each). The combined organic phase was washed with brine (2×30.0 mL) then dried over anhydrous Na₂SO₄, filtered, and concentrated under vacuum. The crude product was purified by column chromatography (SiO₂, petroleum ether: ethyl acetate=15:1 to 1:1, petroleum ether: ethyl acetate=2:1, R_{f}=0.70) to obtain 2-methyl-3-(thiophen-2-yl(tosyl)methyl)-1H-indole (I-2) (700 mg, 1.52 mmol, 32.9% yield) as a yellow solid.

¹H-NMR of 1-2: (400 MHz, DMSO-d₆) δ 11.1 (s, 1H), 7.39 (d, J=8.0 Hz, 1H), 7.52 (dd, J=5.2, 1.2 Hz, 1H), 7.42 (d, J=8.4 Hz, 2H), 7.28 (dt, J=3.6, 1.2, 0.8 Hz, 1H), 7.22 (d, J=8.0 Hz, 3H), 7.03-6.97 (m, 2H), 6.96-6.91 (m, 1H), 6.22 (d, J=0.8 Hz, 1H), 2.31 (s, 3H), 2.04 (s, 3H).

### Alternate Synthesis of Compound 1-2

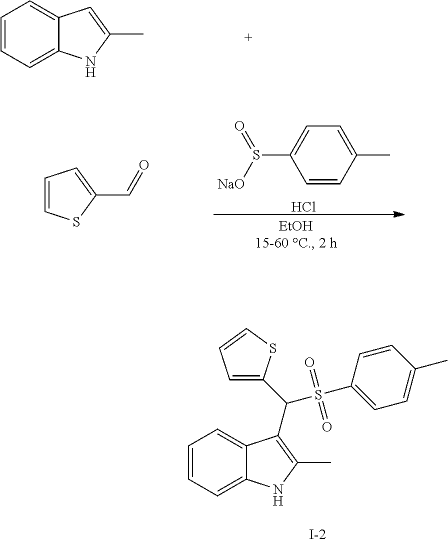

To a solution of HCl (12.0 M, 6.80 mL, 2.42 eq) in EtOH (50.0 mL) was added 2-methyl indole (5.00 g, 38.1 mmol, 1.13 eq) followed by thiophene-2-carbaldehyde (3.15 mL, 33.7 mmol, 1.00 eq) and sodium 4-methylbenzenesulfinate (7.21 g, 40.4 mmol, 1.20 eq) under N₂ at 15° C. The mixture was heated to 60° C. and stirred for 2 h then cooled to room temperature and extracted with ethyl acetate (3×200 mL). The combined organic phase was washed with brine (2×100 mL), dried over anhydrous Na₂SO₄, filtered, and concentrated under vacuum. The crude product was purified by column chromatography (SiO₂, R_{f}=0.40, petroleum ether/ethyl acetate=1/0 to 2/1) to obtain 2-methyl-3-(thiophen-2-yl(tosyl)methyl)-1H-indole (I-2) (6.87 g, 13.7 mmol, 40.7% yield) as a red solid.

¹H-NMR of 1-2: (400 MHz, DMSO). δ 11.0 (s, 1H), 7.73 (s, 1H), 7.52 (d, J=0.8 Hz, 1H), 7.43 (d, J=8.0 Hz, 2H), 7.28 (d, J=3.2 Hz, 1H), 7.22 (d, J=8.0 Hz, 3H), 7.02 - 6.93 (m, 3H), 6.22 (s, 1H), 2.32 (d, J=12.4 Hz, 3H), 2.04 (s, 3H).

### Synthesis of Compound U1

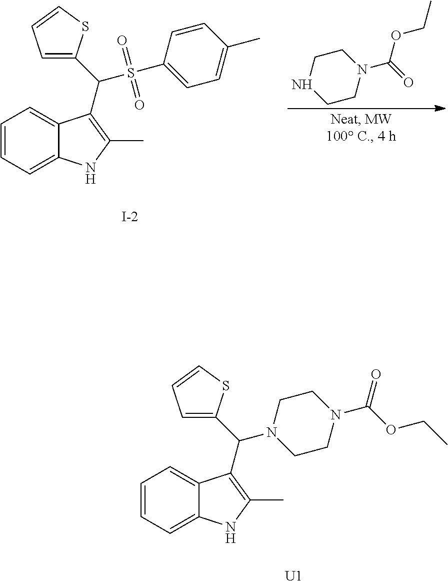

A mixture of I-2 (150 mg, 393 μmol, 1.00 eq) and ethyl piperazine-1-carboxylate (926 μL, 6.32 mmol, 16.1 eq) was heated to 100° C. in a microwave reactor. The mixture was stirred for 4 h then cooled to room temperature and diluted with MeOH (2.00 mL). A solid was collected by filtration and purified by prep-HPLC (column, waters Xbridge C18 150*50 mm*10 μm; mobile phase: [water (NH₄HCO₃)-ACN]; B %: 27%-57%, 10 min) then further purified by pre-HPLC (column: Welch ultimate XB-SiOH 250*50*10 μm; mobile phase: [Hexane-EtOH]; B %: 1%-30%, 15 min) to obtain ethyl 4-((2-methyl-1H-indol-3-yl)(thiophen-2-yl)methyl)piperazine-1-carboxylate (U1) (12.4 mg, 23.5 μmol, 5.98% yield) as a light-yellow solid.

¹H-NMR of U1: (400 MHz, DMSO+D₂O) δ 7.69 (d, J=7.6 Hz, 1H), 7.28-7.20 (m, 2H), 6.97-6.85 (m, 4H), 4.95 (s, 1H), 4.00-3.94 (q, J=7.2 Hz, 2H), 3.40-3.20 (m, 4H), 2.40-2.30 (m, 7H), 1.13-1.09 (t, J=7.2 Hz, 3H).

¹³C NMR of U1: (100 MHz, DMSO+D₂O) Δ 155.2, 148.3, 135.5, 133.5, 126.9, 126.6, 125.1, 124.9, 120.7, 120.0, 119.0, 110.0, 62.7, 61.3, 51.4, 44.1, 15.0, 12.2.

### Synthesis of Compound U6

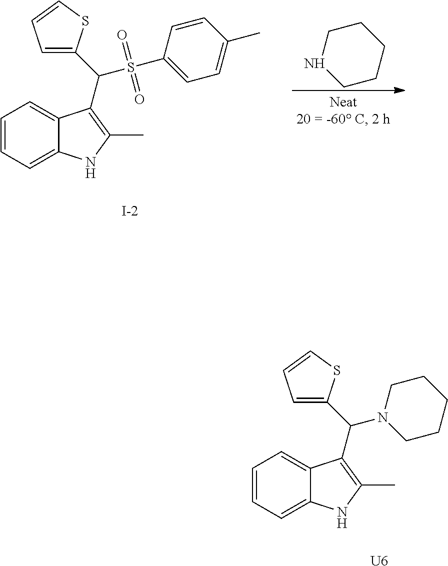

Compound I-2 (100 mg, 235 μmol, 1.00 eq) and piperidine (0.50 mL, 5.1 mmol, 21 eq) were mixed in a 100 mL three neck-bottomed flask at 20° C. and then heated to 60° C. for 2 h. The mixture was cooled to room temperature, diluted with MeOH (2.00 mL), and purified by pre-HPLC (column: Welch Xtimate C18 150*25 mm*5 μm; mobile phase: [water (NH₃H₂O)-ACN]; B %: 50%-80%, 8 min) to obtain compound U6 (53.12 mg, 171 μmol, 72.7% yield,) as a yellow solid.

¹H-NMR of U6: (400 MHz, DMSO) δ 10.8 (s, 1H), 7.72 (d, J=8.00 Hz, 1H), 7.27-7.20 (m, 2H), 6.97-6.83 (m, 4H), 4.90 (s, 1H), 2.38 (d, J=25.6 Hz 7H), 1.43 (d, J=58.8 Hz, 6H).

¹³C-NMR of U6: (100 MHz, DMSO) δ 149.6, 135.7, 133.3, 127.3, 126.4, 124.7, 124.2, 120.4, 120.2, 118.7, 110.8, 110.6, 63.4, 52.8, 26.4, 24.7, 12.4.

### Synthesis of compound U7

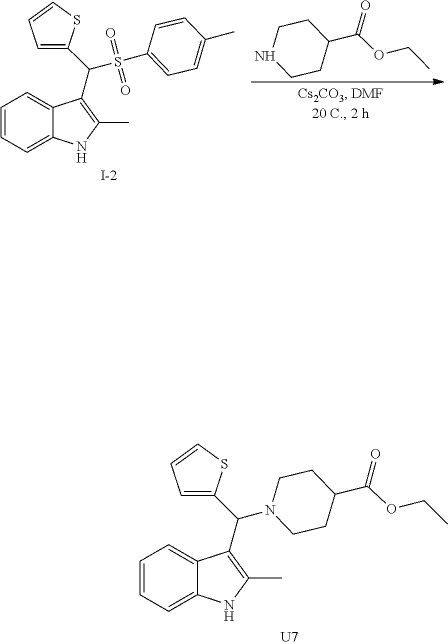

To a solution of compound I-2 (100 mg, 235 μmol, 1.00 eq) in DMF (1.00 mL) at 20° C. was added iso-nipecotic acid ethyl ester (72.5 μL, 470 μmol, 2.00 eq) followed by Cs₂CO₃ (230 mg, 706 μmol, 3.00 eq). The mixture was stirred for 2 h at 20° C. then diluted with EtOH (2.00 mL) and purified by reverse phase HPLC (column: Welch Xtimate C18 150*25 mm*5 μm; mobile phase: [water (NH₃H₂O)-ACN]; B %: 50%-80%, 8 min) to obtain ethyl 1-((2-methyl-1H-indol-3-yl)(thiophen-2-yl)methyl)piperidine-4-carboxylate (U7) (12.8 mg, 33.4 μmol, 14.2% yield) as a brown solid.

¹H-NMR of U7: (400 MHz, DMSO) δ 10.8 (s, 1H), 7.71 (d, J=7.60 Hz, 1H), 7.27, 7.21 (dd, J=5.20, 7.60 Hz, 2H), 6.97-6.92 (m, 3H), 6.91-6.84 (m, 1H), 4.92 (s, 1H), 4.07-4.01 (m, 2H), 2.91 (d, J=4.40 Hz, 2H), 2.32 (d, J=58.0 Hz, 3H), 2.00 (s, 1H), 1.85 (s, 1H), 1.81 (d, J=13.2 Hz, 2H), 1.73 (d, J=12.0 Hz, 1H), 1.63-1.58 (m, 2H).

¹³C-NMR of U7: (100 MHz, DMSO) δ 175.0, 149.3, 135.7, 133.2, 127.1, 126.5, 125.0, 124.4, 120.5, 120.1, 118.8, 110.8, 110.5, 62.4, 60.2, 51.8, 50.6, 28.8, 14.6, 12.4.

### Synthesis of Compound U8

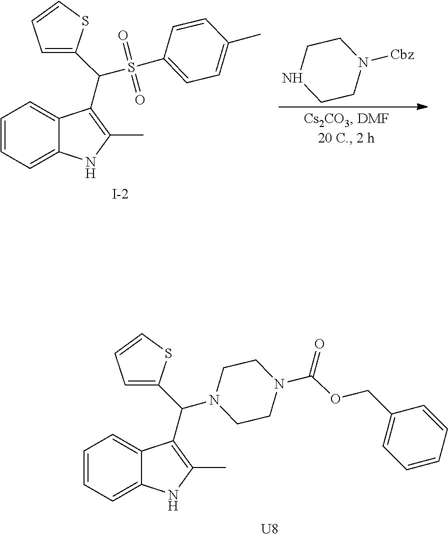

To a solution of compound I-2 (100 mg, 235 μmol, 1.00 eq) in DMF (1.00 mL) at 20° C. was added cbz-piperazine (90.9 μL, 470 μmol, 2.00 eq) followed by Cs₂CO₃ (230 mg, 706 μmol, 3.00 eq). The mixture was stirred for 2 h at 20° C. then diluted with MeOH (2.00 mL) and purified by prep-HPLC (column: Welch Xtimate C18 150*25 mm*5 μm; mobile phase: [water (NH₃H₂O)-ACN]; B %: 50%-80%, 8 min) to obtain benzyl 4-((2-methyl-1H-indol-3-yl)(thiophen-2-yl)methyl)piperazine-1-carboxylate (U8) (92.0 mg, 144 μmol, 61.4% yield) as a pink solid.

¹H-NMR of U8: (400 MHz, DMSO) δ 10.8 (s, 1H), 7.73 (d, J=7.6 Hz, 1H), 7.35-7.32 (m, 6H), 7.31 (d, J=2.8 Hz, 1H), 7.29-6.97 (m, 3H), 6.92 (d, J=38.8 Hz, 1H), 5.04 (s, 2H), 4.97(s, 1H), 3.41 (s, 5H), 2.42-2.32 (m, 7H).

¹³C-NMR of U-8: (100 MHz, DMSO) δ 154.8, 148.4, 137.4, 135.7, 133.6, 128.8, 128.2, 127.9, 127.0, 126.6, 125.2, 124.8, 120.6, 120.0, 118.9, 110.9, 110.0, 66.5, 62.7, 51.5, 44.2, 12.4.

### Synthesis of Compound U9

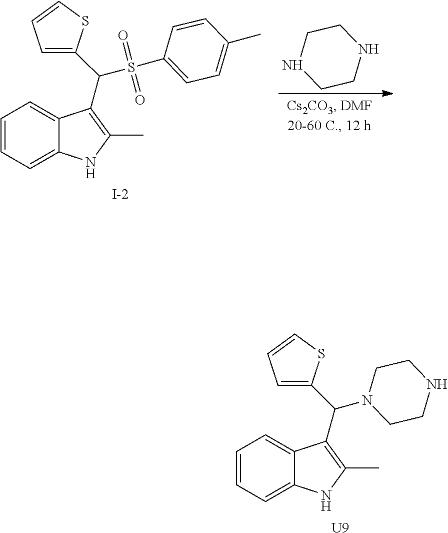

To a solution of piperazine (160 μL, 1.63 mmol, 10.0 eq) in DMF (1.00 mL) at 20° C. was added I-2 (100 mg, 162 μmol, 1.00 eq) followed by Cs₂CO₃ (159 mg, 488 μmol, 3.00 eq). The mixture was heated to 60° C. and stirred for 12 h. The reaction mixture was cooled to room temperature, diluted with MeOH (1.00 mL), and purified by pre-HPLC (column: Welch Xtimate C18 150*25 mm*5 μm; mobile phase: [water (NH₃H₂O)-ACN]; B %: 50%-80%, 8 min) to obtain 2-methyl-3-(piperazin-1-yl(thiophen-2-yl)methyl)-1H-indole (U9) (30 mg, 54.3 μmol, 33.3% yield) as a yellow solid.

¹H-NMR of U9: (400 MHz, DMSO) δ 10.9-10.8 (m, 1H), 7.66 (d, J=8.0 Hz, 1H), 7.23- 7.19 (m, 2H), 6.95-6.88 (m, 3H), 6.81-6.79 (m, 1H), 4.94 (d, J=3.2 Hz, 1H), 2.64-2.57 (m, 4H), 2.40 (d, J=3.2 Hz, 7H).

¹³C-NMR of U9: (400 MHz, DMSO) δ 148.9, 135.6, 133.4, 127.1, 126.4, 124.9, 124.5, 120.5, 120.1, 118.8, 117.1, 110.8, 110.2, 107.4, 62.8, 12.3.

### Synthesis of Compound U10

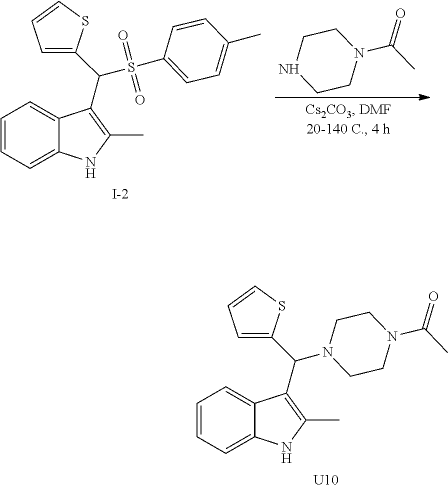

To a solution of I-2 (100 mg, 162 μmol, 1.00 eq) in DMF (1.00 mL) at 20° C. was added 1-(piperazin-1-yl)ethan-1-one (41.7 mg, 325 μmol, 2.00 eq) followed by Cs₂CO₃ (159 mg, 488 μmol, 3.00 eq). The mixture heated to 140° C. and stirred for 4 h then cooled to room temperature and diluted with MeOH (1.00 mL). The mixture was purified by pre-HPLC (column: Welch Xtimate C18 150*25 mm*5 μm; mobile phase: [water (NH₃H₂O)-ACN]; B %: 50%-80%, 8 min) to obtain 1-(4-((2-methyl-1H-indol-3-yl)(thiophen-2-yl)methyl)piperazin-1-yl)ethan-1-one (U10) (12.7 mg, 28.3 μmol, 17.4% yield) as a pink solid.

¹H-NMR of U10: (400 MHz, DMSO) δ 10.9 (s, 1H), 7.73 (d, J=7.6 Hz, 1H), 7.31 (d, J=1.2 Hz, 1H), 7.22 (d, J=7.6 Hz, 1H), 6.97 (d, J=3.2 Hz, 2H), 6.87 - 6.86 (m, 2H), 4.97 (s, 1H), 3.44-3.41 (m, 4H), 2.45-2.35 (m, 7H), 1.93 (s, 3H).

¹³C-NMR of U10: (400 MHz, DMSO) δ 168.5, 148.4, 133.6, 133.2, 127.0, 126.6, 125.2, 125.0, 120.6, 120.0, 118.9, 110.9, 110.0, 62.7, 52.0, 51.5, 21.6, 12.4.

### Synthesis of Compound U11

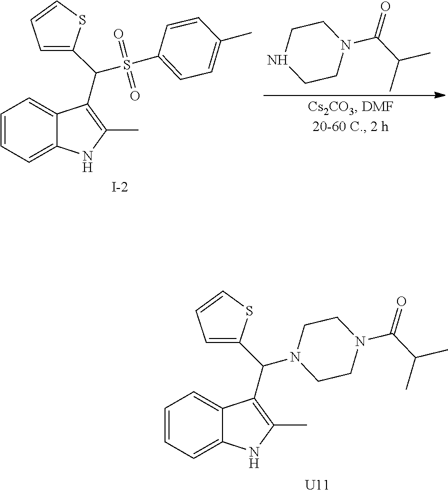

To a solution of 2-methyl-1-(piperazin-1-yl)propan-1-one (101 mg, 651 μmol, 2.00 eq) in DMF (1.00 mL) at 15° C. was added Cs₂CO₃ (318 mg, 976 μmol, 3.00 eq) followed by dropwise addition of I-2 (200 mg, 325 μmol, 1.00 eq) as a solution in in DMF (2.00 mL). The mixture was heated to 60° C. and stirred for 2 h. The reaction mixture was cooled to 15° C., filtered, and the filtrate purified by pre-HPLC (column: Welch Xtimate C18 150*25 mm *5 μm; mobile phase: [water (NH₃H₂O)-ACN]; B %: 50%-80%, 8 min) to obtain 2-methyl-1-(4-((2-methyl-1H-indol-3-yl)(thiophen-2-yl)methyl)piperazin-1-yl)propan-1-one (Ull) (72.9 mg, 170 μmol, 52.3% yield) as a yellow solid.

¹H-NMR of U11: (400 MHz, DMSO) δ 10.7 (s, 1H), 7.76 (d, J=8.0 Hz, 1H), 7.30 (d, J=5.2 Hz, 1H), 7.22 (d, J=8.0 Hz, 1H), 6.98-6.93 (m, 3H), 6.92-6.86 (m, 1H), 4.95 (s, 1H), 3.48 (d, J=8.0 Hz, 4H), 2.82-2.75 (m, 1H), 2.38 (d, J=21.6 Hz, 7H), 0.944-0.928 (m, 6H).

¹³C-NMR of Ull: (100 MHz, DMSO) δ 174.5, 148.4, 135.7, 133.5, 126.9, 126.5, 125.2, 124.8, 120.6, 120.0, 118.9, 110.9, 110.2, 62.8, 52.2, 51.7, 29.3, 19.8, 12.3.

### Synthesis of Compound U12

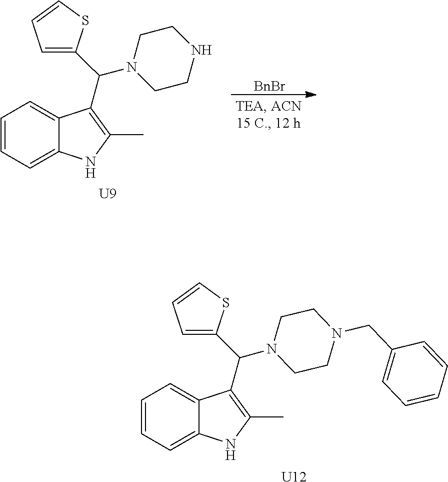

To a solution of U9 (60 mg, 192 μmol, 1.00 eq) in ACN (1.00 mL) at 15° C. was added bromo-benzene (45.7 μ,L, 385 μmol, 2.00 eq) followed by TEA (80.4 μL, 577 μmol, 3.00 eq). The mixture was stirred for 12 h at 15° C. then filtered and purified by pre-HPLC (column: Welch Xtimate C18 150*25 mm*5 μm; mobile phase: [water (NH₃H₂O)-ACN]; B %: 50%-80%, 8 min) to obtain 3-((4-benzylpiperazin-1-yl)(thiophen-2-yl)methyl)-2-methyl-1H-indole (U12) (3.98 mg, 9.59 μmol, 4.98% yield,) as a yellow gum.

¹H-NMR of U12: (400 MHz, DMSO) δ 10.8 (s, 1H), 7.71 (d, J=7.60 Hz, 1H), 7.28-7.19 (m, 7H), 6.94-6.84 (m, 4H), 4.92 (s, 1H), 3.44 (s, 2H), 2.52-2.32 (m, 11H).

¹³C-NMR of U12: (100 MHz, DMSO) δ 148.9, 138.7, 135.7, 133.3, 129.2, 128.6, 127.3, 127.1, 126.4, 124.9, 124.5, 120.5, 120.1, 118.7, 110.8, 110.4, 62.9, 62.5, 53.5, 51.7, 12.4.

### Synthesis of Compound U13

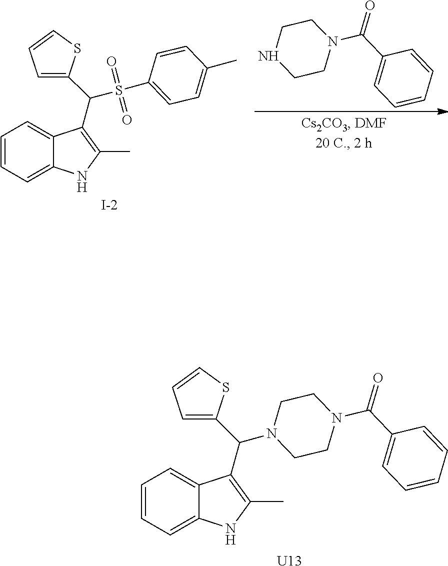

To a solution of I-2 (100 mg, 162 μmol, 1.00 eq) in DMF (1.00 mL) at 20° C. was added phenyl(piperazin-1-yl)methanone (61.9 mg, 325 μmol, 2.00 eq) followed by Cs₂CO₃ (159 mg, 488 μmol, 3.00 eq). The mixture was stirred for 2 h at 20° C. then diluted with MeOH (1.00 mL) and purified by pre-HPLC (column: Welch Xtimate C18 150*25 mm*5 μm; mobile phase: [water (NH₃H₂O)-ACN]; B %: 50%-80%, 8 min) to obtain (4-((2-methyl-1H-indol-3-yl)(thiophen-2-yl)methyl)piperazin-1-yl)(phenyl)methanone (U13) (37.6 mg, 87.2 μmol, 53.5% yield) as a yellow solid.

¹H-NMR of U13: (400 MHz, DMSO) δ 10.9 (s, 1H), 7.73 (d, J=8.0 Hz, 1H), 7.40-7.20 (m, 7H), 6.97-6.86 (m, 4H), 5.00 (s, 1H), 3.64-3.57 (m, 4H), 2.41 (s, 7H).

¹³C-NMR of U13: (400 MHz, DMSO) δ 169.2, 148.3, 136.3, 135.7, 133.6, 129.9, 128.8, 127.4, 127.0, 126.6, 125.2, 124.9, 120.6, 120.0, 118.9, 110.9, 62.7, 51.7, 12.4.

### Synthesis of Compound U14

### Step 1

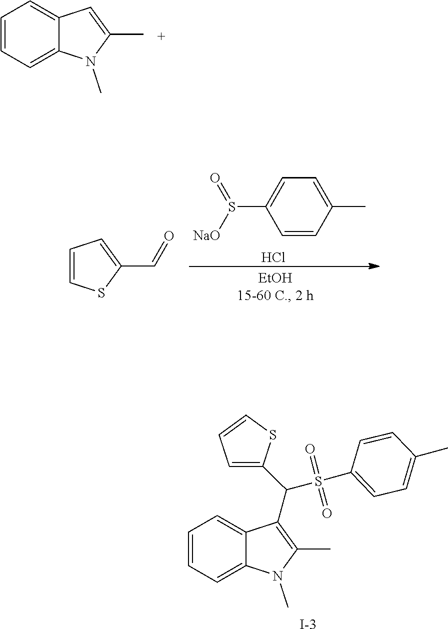

To a solution of HCl (12.0 M, 614 μ,L, 2.42 eq) in EtOH (25.0 mL) at 15° C. was added 1,2-dimethyl-1H-indole (500 mg, 3.44 mmol, 1.13 eq), thiophene-2-carbaldehyde (284 μL, 3.05 mmol, 1.00 eq) and sodium 4-methylbenzenesulfinate (651 mg, 3.66 mmol, 1.20 eq). The mixture was heated to 60° C. and stirred for 2 h. The reaction mixture was cooled to room temperature, diluted with water (100 mL) and extracted with ethyl acetate (3×100 mL). The combined organic phase was washed with brine (100 mL), dried over anhydrous Na₂SO₄, filtered, and concentrated under vacuum to obtain 1,2-dimethyl-3-(thiophen-2-yl(tosyl)methyl)-1H-indole (I-3) (1.10 g, 1.52 mmol, 49.7% yield) as a red solid.

¹H-NMR of 1-3: (400 MHz, DMSO) δ 7.74 (d, J=8.0 Hz, 1H), 7.51 (d, J=4.0 Hz, 1H), 7.44 (d, J=8.0 Hz, 2H), 7.37 (d, J=8.0 Hz, 1H), 7.27-7.20 (m, 3H), 7.10-7.06 (m, 1H), 7.02-6.91 (m, 2H), 3.56 (s, 3H), 2.34-2.30 (s, 3H), 2.12 (s, 3H).

### Step 2

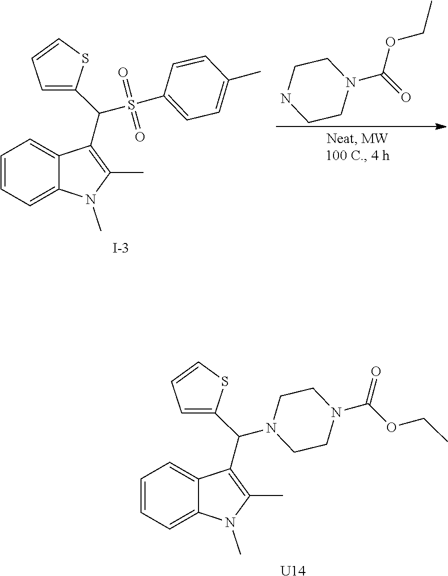

To a solution of I-3 (1.03 g, 1.42 mmol, 1.00 eq) in DMF (10.00 mL) at 25° C. was added ethyl piperazine-1-carboxylate (414 μL, 2.83 mmol, 2.00 eq) followed by Cs₂CO₃ (1.38 g, 4.25 mmol, 3.00 eq). The mixture was stirred for 2 h at 25° C. then filtered and purified by pre-HPLC (column: Phenomenex C18 250*50 mm*10 μm; mobile phase: [water (ammonia hydroxide v/v)-ACN]; B %: 52%-82%, 10 min) to obtain ethyl 4-((1,2-dimethyl-1H-indol-3-yl)(thiophen-2-yl)methyl)piperazine-1-carboxylate (U14) (59.7 mg, 150 μmol, 10.6% yield) as an off-white solid.

¹H-NMR of U14: (400 MHz, DMSO) δ 7.79 (d, J=7.6 Hz, 1H), 7.34 (d, J=8.4 Hz, 1H), 7.30 (dd, J=6.4, 1.2 Hz, 1H), 7.05-7.03 (m, 1H), 6.94-7.00 (m, 2H), 6.85 (m, 1H), 5.03 (s, 1H), 3.98 (q, J=0.4 Hz, 2H), 3.62 (s, 3H), 3.36 (d, J=5.6 Hz, 6H), 2.44 (s, 3H), 2.36 - 2.30 (m, 2H), 1.14-1.11 (m, 3H).

¹³C-NMR of U14: (100 MHz, DMSO) δ 155.0, 148.4, 135.7, 133.8, 135.0, 126.5, 126.0, 125.2, 124.9, 120.7, 120.2, 119.0, 110.1, 109.5, 63.0, 61.1, 51.5, 44.0, 29.8, 15.0, 10.9.

### Synthesis of Compound U15

### Step 1

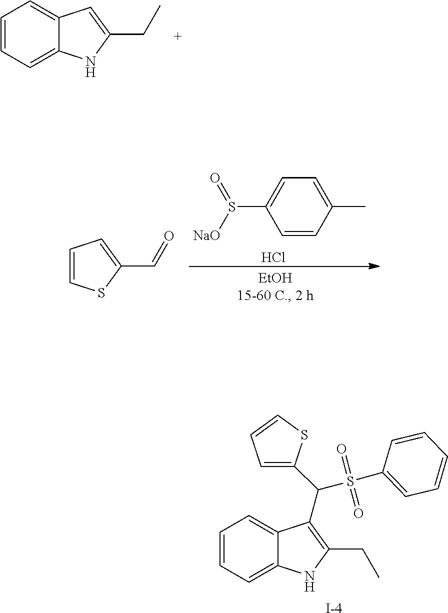

To a solution of HCl (12.0 M, 122 μL, 2.42 eq) in EtOH (5.00 mL) at 15° C. was added 2-ethyl-1H-indole (100 mg, 688 μmol, 1.13 eq) followed by thiophene-2-carbaldehyde (5a) (56.9 μL, 609 μmol, 1.00 eq) and sodium 4-methylbenzenesulfinate (130 mg, 731 μmol, 1.20 eq). The mixture was heated to 60° C. and stirred for 2 h. Upon cooling to 25° C., water (20.0 mL) was added and then the mixture was extracted with ethyl acetate (2×20.0 mL). The combined organic phase was washed with brine (20.0 mL), dried over anhydrous Na₂SO₄, filtered, and concentrated under vacuum to obtain 2-ethyl-3-(thiophen-2-yl(tosyl)methyl)-1H-indole (I-4) (89.0 g, 2.25 μmol, 36.9% yield) as a red solid.

¹H-NMR of I-4: (400 MHz, d4-MeOD) δ 7.78 (d, J=8.0 Hz, 1H), 7.39-7.35 (m, 4H), 7.25 (d, J=8.0 Hz, 1H), 7.14 (d, J=8.0 Hz, 2H), 7.07-7.03 (m, 1H), 7.02 - 6.94 (m, 2H), 6.02 (s, 1H), 2.57-2.47 (m, 1H), 2.41-2.37(m, 1H), 2.33 (s, 3H), 0.98-0.95 (m, 3H).

### Step 2

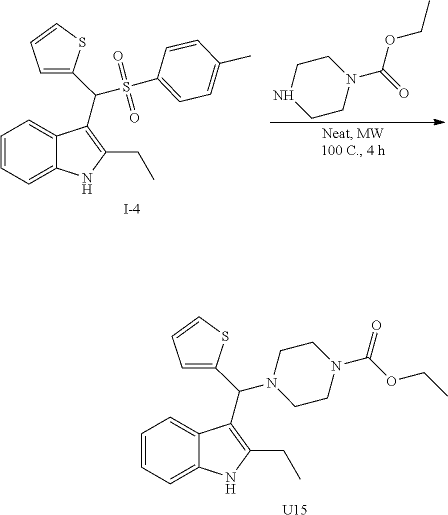

To a solution of I-4 (89.0 mg, 225 μmol, 1.00 eq) in DMF (2.00 mL) at 15° C. was added ethyl piperazine-1-carboxylate (65.9 μL, 450 μmol, 2.00 eq) followed by Cs₂CO₃ (219 mg, 675 μmol, 3.00 eq). The mixture was stirred for 2 h at 15° C. then filtered and the filtrate purified by pre-HPLC (column: Welch Xtimate C18 150*25 mm*5 μm; mobile phase: [water (NH₃H₂O)-ACN]; B %: 50%-80%, 8 min) to obtain ethyl 4-((2-ethyl-1H-indol-3-yl)(thiophen-2-yl)methyl)piperazine-1-carboxylate (U15) (19.2 mg, 68.2 μmol, 30.3% yield) as a white solid.

¹H-NMR of U15: (400 MHz, DMSO) δ 10.8 (s, 1H), 7.80 (d, J=8.0 Hz, 1H), 7.29 (d, J=5.2 Hz, 1H), 7.23 (d, J=8.0 Hz, 1H), 7.02-6.89 (m, 3H), 6.88-6.83 (m, 1H), 4.95 (s, 1H), 4.03-3.95 (m, 2H), 2.85-2.79 (m, 2H), 2.67 (s, 2H), 2.41-2.31 (m, 6H), 1.20 (t, J=7.6 Hz, 3H), 1.13 (t, J=7.2 Hz, 3H).

¹³C-NMR of U15: (100 MHz, DMSO) δ 155.0, 148.6, 139.2, 135.9, 126.6, 126.5, 125.1, 124.9, 120.7, 120.4, 118.8, 111.0, 109.5, 62.8, 61.1, 51.6, 44.1, 19.5, 15.0, 14.7.

### Synthesis of Compound U16

### Step 1

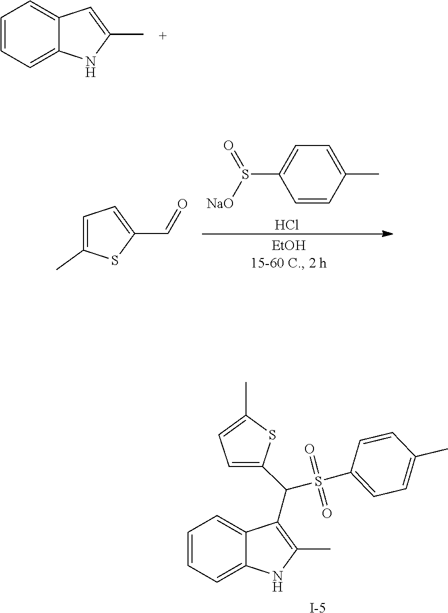

To a mixture of EtOH (5.00 mL) and HCl (12.0 M, 136 μL, 2.42 eq) at 15° C. was added sodium 4-methylbenzenesulfinate (144 mg, 809 μmol, 1.20 eq) followed by 2-methyl indole (100 mg, 762 μmol, 1.13 eq) and 5-methylthiophene-2-carbaldehyde (73.4 μL, 675 μmol, 1.00 eq). The mixture was heated to 60° C. and stirred for 2 h. Upon cooling to 15° C., water (20.0 mL) was added, and the mixture was extracted with ethyl acetate (3×20.0 mL). The combined organic phase was washed with brine (50.0 mL), dried over anhydrous Na₂SO₄, filtered, and concentrated under vacuum. The crude product was purified by pre-TLC (Petroleum ether: ethyl acetate=2:1, R_{f}=0.37) to obtain 2-methyl-3-((5-methylthiophen-2-yl)(tosyl)methyl)-1H-indole (I-5) (110 mg, 278 μmol, 41.2% yield) as a red solid.

¹H-NMR of I-5: (400 MHz, CDCl₃) δ 7.87 (s, 1H), 7.69 (br d, J=8.0 Hz, 1H), 7.41 (br d, J=8.0 Hz, 2H), 7.24 (br d, J=8.4 Hz, 2H), 7.16-7.02 (m, 7H), 6.63 (br d, J=1.6 Hz, 1H), 5.75 (s, 1H), 2.46 (s, 3H), 2.33 (s, 3H).

### Step 2

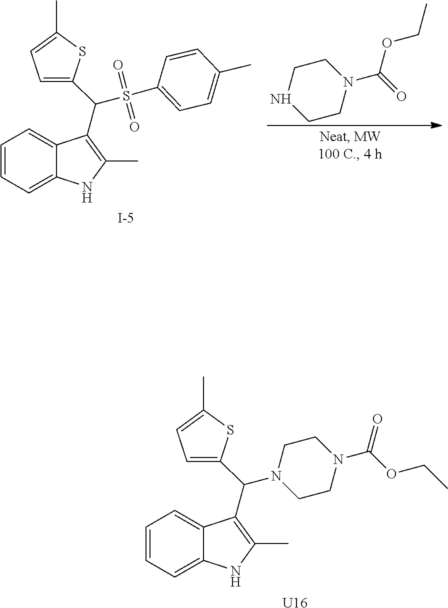

To a solution of I-5 (110 mg, 278 μmol, 1.00 eq) in DMF (2.00 mL) at 15° C. was added ethyl piperazine-1-carboxylate (81.5 μL, 556 μmol, 2.00 eq) followed by Cs₂CO₃ (272 mg, 834 μmol, 3.00 eq). The mixture was stirred for 2 h at 15° C. then filtered and the filtrate purified by pre-HPLC (column: Welch Xtimate C18 150*25 mm*5 μm; mobile phase: [water (NH₃H₂O)-ACN]; B %: 55%-85%, 8 mins) to obtain ethyl 4-((2-methyl-1H-indol-3-yl)(5-methylthiophen-2-yl)methyl)piperazine-1-carboxylate (U16) (9.85 mg, 21.4 μmol, 7.72% yield) as a yellow solid.

¹H-NMR of U16: (400 MHz, CDCl₃) δ 7.98-7.91 (d, J=8.4 Hz, 1H), 7.83 (s, 1H), 7.26-7.22 (dd, J=1.2, 1.6 Hz, 1H), 7.10 (dt, J=1.6, 6.8 Hz, 2H), 6.73 (d, J=3.6 Hz, 1H), 6.51 -6.46 (m, 1H), 4.80 (s, 1H), 4.11 (q, J=7.2 Hz, 2H), 3.48 (br s, 4H), 2.55 - 2.47 (m, 3H), 2.45 (s, 4H), 2.38 (d, J=0.8 Hz, 3H), 1.23 (t, J=7.2 Hz, 3H).

¹³C-NMR of U16: (100 MHz, CDCl₃) δ 155.5, 145.2, 139.0, 135.2, 132, 127, 124.2, 123.9, 121.2, 120.6, 119.3, 111.5, 110.1, 63.5, 61.2, 51.5,44.0, 15.4, 14.7, 12.4.

### Synthesis of Compound U17

### Step 1

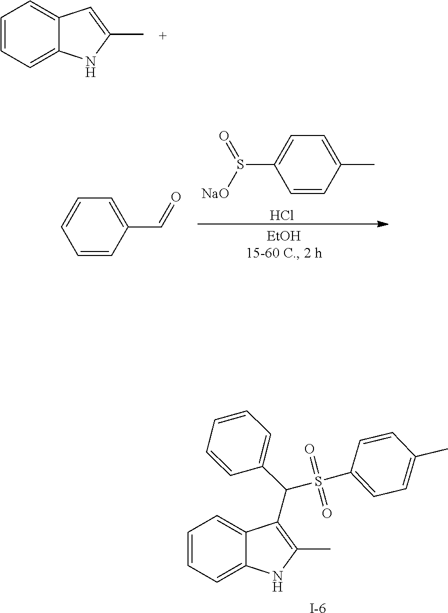

To a mixture of EtOH (5.00 mL) and HCl (12.0 M, 136 μL, 2.42 eq) at 15° C. was added sodium 4-methylbenzenesulfinate (144 mg, 809 μmol, 1.20 eq) followed by 2-methyl indole (100 mg, 762 μmol, 1.13 eq) and benzyaldehyde (68.2 μL, 675 mol, 1.00 eq). The mixture was heated to 60° C. and stirred for 2 h. Upon cooling to 15° C., water (20.0 mL) was added, and the mixture was extracted with ethyl acetate (3×20.0 mL). The combined organic phase was washed with brine (50.0 mL), dried over anhydrous Na₂SO₄, filtered, and concentrated under vacuum. The crude product was purified by pre-TLC (Petroleum ether: ethyl acetate=1:0 to 2:1, R_{f}=0.35) to obtain 2-methyl-3-(phenyl(tosyl)methyl)-1H-indole (I-6) (135 mg, 87.7 μmol, 13.0% yield) as a red solid.

¹H-NMR of I-6: (400 MHz, CDCl₃) δ 7.85 (br s, 1H), 7.82-7.75 (m, 3H), 7.43 (d, J=8.0 Hz, 2H), 7.33 (br d, J=7.2 Hz, 3H), 7.23 (br d, J=8.2 Hz, 2H), 7.16-7.09 (m, 2H), 7.07 (d, J=7.6 Hz, 3H), 5.65 (s, 1H), 4.13 (q, J=7.2 Hz, 1H), 2.33 (s, 3H), 2.10 (s, 3H), 2.06 (s, 1H), 1.27 (t, J=7.2 Hz, 1H).

### Step 2

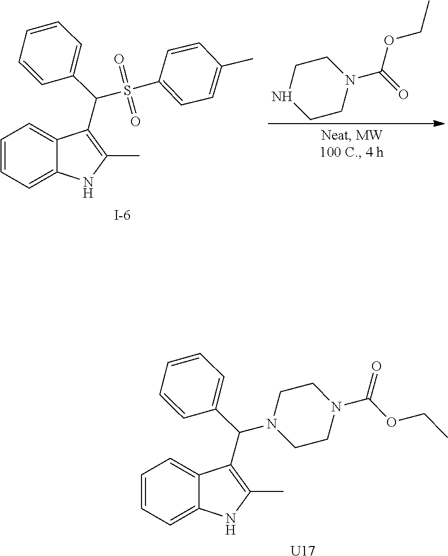

To a solution of I-6 (135 mg, 87.7 μmol, 1.00 eq) in DMF (2.00 mL) at 15° C. was added ethyl piperazine-1-carboxylate (25.7 μL, 175 μmol, 2.00 eq) followed by Cs₂CO₃ (85.7 mg, 263 μmo, 3.00 eq). The mixture was stirred for 2 h at 15° C. then filtered and the filtrate purified by pre-HPLC (column: Welch Xtimate C18 150*25 mm*5 μm; mobile phase: [water (NH₃H₂O)-ACN]; B %: 55%-85%, 8 min) to obtain ethyl 4-((2-methyl-1H-indol-3-yl)(phenyl)methyl)piperazine-1-carboxylate (U17) (37.7 mg, 92.5 μmol) as a yellow solid.

¹H-NMR of U17: (400 MHz, CDCl₃) δ 8.05-7.99 (m, 1H), 7.76 (br s, 1H), 7.53 (d, J=7.2 Hz, 2H), 7.26-7.20 (m, 3H), 7.16 (d, J=7.6 Hz, 1H), 7.11-7.07 (m, 2H), 4.54 (s, 1H), 4.12 (q, J=7.2 Hz, 2H), 3.50 (br s, 4H), 2.52-2.48 (m, 1H), 2.46 (s, 4H), 2.40 (br s, 2H), 1.24 (t, J=7.2 Hz, 3H).

¹³C-NMR of U17: (400 MHz, CDCl₃) δ 155.6, 143.2, 135.3, 131.7, 128.4, 127.6, 127.3, 126.6, 121.1, 120.3, 119.5, 112.5, 110.1, 68.4, 61.2, 51.9, 44.0, 14.7, 12.5.

### Example 2: Biological Assays

Preliminary structure activity relationship (SAR) analyses of selected small molecules revealed molecular features that influenced Them1 enzymatic activity and binding interactions. In cultured primary brown adipocytes and hepatocytes, the most promising inhibitors reversed the suppressive effects of Them1 on oxygen consumption rates (OCR), a key surrogate measure of thermogenesis.

### Animals

Generation of conditional transgenic tissue-specific Them1 overexpression (wild type) mice was previously described with modifications (Madisen et al., 2010). A cDNA encoding mouse Them1 was fused to a C-terminal FLAG-tag (FLAG) as a marker for Cre recombination and cloned into a Rosa26 expression vector consisting of a CMV promoter, a STOP cassette flanked by loxP sites and a polyA tail (pA). The plasmid was then linearized and microinjected into pronuclei of eggs from C57BL/6 female mice (Mouse Genetics Core Facility; Memorial Sloan Kettering Cancer Center; New York, NY). Conditional transgenic adipose tissue-specific Them1 overexpression (A-Them1 Tg) mice were generated by crossing wild type mice to transgenic mice expressing Cre recombinase driven by the adiponectin gene promoter (B6.FVB-Tg(Adipoq-Cre)1Evdr/J; Strain #: 028020; Jackson Laboratory; Bar Harbor, ME) on a congenic C57/BL6 background (Eguchi et al., 2011). Mice with liver-specific Them1 overexpression (L-Them1Tg) and wild type controls, along with Them1 knockout (Them1^{-/-}) controls were generated by intravenously injecting 6- to 14-w old wild type, C57BL/6 and Them1^{-/-} mice (Zhang et al., 2012), respectively, with adeno-associated virus 8 (AAV8) expressing Cre recombinase driven by the human thyroid hormone-binding globulin (TBG) promoter (AAV8.TBG.Cre). Adiponectin- and TBG-Cre mediated recombination resulted in excision of the STOP cassette to bring Them1 under control of the CMV promoter in adipose tissue and hepatocytes, respectively. Litters were genotyped for integration of the Them1 transgene by PCR analysis from ear genomic DNA. Tissues were harvested, immediately snap frozen in liquid nitrogen and stored at −80° C. Mice were same sex housed in mixed genotype groups (3 to 5 mice per cage) in a barrier facility on a 12 h light/dark cycle. Animal use and euthanasia protocols were performed using approved guidelines by Weill Cornell Medical College.

### Cultures of Primary Brown Adipocytes

Primary brown adipocytes from 4- to 6-w old wild type and A-Them1Tg mice were isolated, cultured and differentiated as described (Okada et al., 2016). In brief, BAT tissues harvested from 5- to 7-w old mice were pooled, minced and digested with collagenase B (Sigma-Aldrich; St. Louis, MO), and dispersed in growth medium [DMEM/F12 containing 4.5 g/L glucose, 0.1 mM pyruvate, 10 mM HEPES (Thermo Fisher; Waltham, MA) supplemented with 1% penicillin/streptomycin, 1% GlutaMAX (Thermo Fisher; Waltham, MA) and 20% fetal bovine serum]. Cells were seeded at 2,000 per well into XF96 cell culture plates (Seahorse Bioscience; North Billerica, MA) pre-coated with rat tail collagen (Sigma Aldrich; St. Louis, MO). Upon achieving confluence, pre-adipocytes were induced to differentiate for 8 d by culturing for the first 48 h in differentiation medium [DMEM/F12 containing 4.5 g/L glucose, 0.1 mM pyruvate, 10 mM HEPES (Thermo Fisher; Waltham, MA) supplemented with 1% penicillin/streptomycin, 1% GlutaMAX (Thermo Fisher; Waltham, MA), 10% fetal bovine serum, bovine insulin (5 μg/mL; Thermo Fisher; Waltham, MA) and rosiglitazone (1 μM; Sigma Aldrich; St. Louis, MO)] supplemented with dexamethasone (5 μM; Sigma Aldrich; St. Louis, MO) and 3-isobutyl-1-methylxanthine (0.5 mM; Sigma Aldrich; St. Louis, MO) and differentiation medium thereafter. On differentiation d 6 to 8, the adipocyte differentiation media was supplemented with L-(−)-norepinephrine (+) bitartrate (Calbiochem; EMD Millipore; Billerica, MA). Primary brown adipocytes were maintained in a cell culture incubator at 37° C. with 5% CO₂.

### Cultures of Primary Hepatocytes

Primary hepatocytes cultured were prepared from 6- to 14-w old wild type, L-Them1Tg and Them1^{-/-} mice following anesthesia with ketamine and xylazine. Livers were perfused through the portal vein with 20 mL of liver perfusion medium (Thermo Fisher; Walham, MA) followed by 40 mL of liver digestion medium (Thermo Fisher; Walham, MA). Primary hepatocytes were gently disrupted from the liver capsule into hepatocyte wash medium (Thermo Fisher; Waltham, MA), filtered through a 70 μm cell strainer and then spun down at 30×g for 4 min at 4° C. Primary hepatocytes were cultured in William's Medium E (Thermo Fisher; Waltham, MA) supplemented with 1% penicillin/streptomycin and 10% fetal bovine serum. For the fatty acid oxidation and glucose production assays, primary hepatocytes were seeded at 2.5×10⁵ cells per well into XF24 cell culture microplates (Seahorse Bioscience; North Billerica, MA) pre-coated with rat tail collagen (Sigma Aldrich; St. Louis, MO) or at 5×10⁵ cells/well in 6-well Primaria plates (Corning Inc.; Corning NY), respectively. Cultured primary hepatocytes were maintained in a cell culture incubator at 37° C. with 5% CO₂.

### Expression and Purification of Recombinant Acot Isoforms

Human Them1 consists of two splice variants (Them1a and Them1b) that are distinguished by an additional 13 amino acids at the C-terminus of the ‘a’ isoform (Adams et al., 2001). For human Them1 (Them1) and a truncated Them1 containing the two thioesterase domains, but lacking the START domain (Them1-ΔSTART), a synthetic gene encoding Them1b (the human ortholog of mouse Them1) was codon optimized to achieve maximal recombinant protein expression (Thermo Fisher; Waltham, MA) and subcloned into a pET19b bacterial expression vector (Novagen, EMD Biosciences; Madison, WI), which introduced an in-frame N-terminal His-tag. Sufficient quantities of recombinant Them1 required for the HTS were obtained as a service of the University of Georgia Bioexpression and Fermentation Core Facility (Athens, GA). Cultures of E. coli Shuffle T7 competent cells (New England Biolabs, Ipswich, MA) transformed with pET19b-Them1 plasmid were grown to an A₆₀₀ of ˜0.5- 0 0.7 in terrific broth followed by induction of recombinant Them1 by the addition of 0.5 mM isoproyl β-D-thiogalactoside with 16 h of shaking (250 rpm) at 18° C. The bacteria were harvested by centrifugation and then lysed with 20 mM Tris-Cl (pH 7.4), 500 mM NaCl, 150 mM imidazole and 1 mM β-mercaptoethanol. The soluble fraction following centrifugation of the bacterial lysate was purified by fast liquid protein chromatography (FPLC) using a HisTrap affinity column (HisTrap HP column; GE Healthcare, Waukesha, WI) after equilibration with 20 mM Tris-Cl (pH 7.4), 500 mM NaCl, 150 mM imidazole and 1 mM β-mercaptoethanol, and then washed with the same buffer. Recombinant Them1 was eluted from the HisTrap HP column using 20 mM Tris-Cl (pH 7.4), 500 mM NaCl, 500 mM imidazole and 1 mM β-mercaptoethanol. To further increase purity, recombinant Them1 was applied to a HisTrap HP affinity column (HisTrap HP column; GE Healthcare, Waukesha, WI) and re-purified as described above followed by dialysis into buffer containing 20 mM Tris-Cl (pH 7.4), 500 mM NaCl and 10% glycerol using a slide-a-lyzer dialysis cassette (Thermo Fisher; Waltham, MA). Purity of recombinant Them1 was analyzed by SDS-PAGE followed by Coomassie Brilliant Blue staining. The concentration of recombinant Them1 was determined according to the molar extinction coefficient at 280 nm, which was calculated based on the amino acid sequence (www.expasy.org). Single use aliquots of recombinant Them1 were stored at −80° C. until use to prevent protein precipitation during purification. The pH values of all buffers were adjusted to remain at least two units removed from the pI of recombinant Them1.

Molecular cloning, and bacterial expression and purification of recombinant Acot isoforms were prepared as previously described with modifications (Han and Cohen, 2012). The open reading frames of mouse Acot1 (Acot1), mouse Them1 and human Acot12 (Acot12) were amplified by PCR using the template plasmids ds-RED-Acot1, pCMV-SPORT6-Them1 and pcDNA-Acot12, respectively. The open reading frames of mouse Acot2 (Acot2) and human Acot13 (Acot13) were amplified by PCR using mouse liver tissue. The genes were subcloned into pET19b or pET29b bacterial expression vectors (Novagen, EMD Biosciences; Madison, WI), which introduced an in-frame N- or C-terminal His-tag, respectively. Maltose-binding protein (MBP)-tagged human Acot9 (Acot9) plasmid was prepared as previously described (Tillander et al., 2014). These plasmids were transformed into E. coli strains BL21 (DE3) (New England Biolabs; Ipswich, MA) or Shuffle T7 competent cells (New England Biolabs, Ipswich, MA), grown to an A600 of 0.5 — 0.7 in terrific broth and then induced by the addition of 0.5 mM isoproyl β-D-thiogalactoside with 16 h of shaking (250 rpm) at 18° C. The bacteria were harvested by centrifugation at 10,000×g for 20 min at 4° C., and then lysed with 20 mM Tris-Cl, (pH 7.4) 500 mM NaCl, 150 mM imidazole, 1 mM β-mercaptoethanol and ethylenediaminetetraacetic acid-free protease inhibitors (Sigma Aldrich, St. Louis, MO). Following centrifugation of the bacterial lysate at 20,000×g for 20 min at 4° C., recombinant Acot isoforms were purified by FPLC using a HisTrap affinity column (HisTrap HP column; GE Healthcare, Waukesha, WI) unless otherwise specified. The soluble fractions containing recombinant Acot isoforms were applied to the column after equilibration with 20 mM Tris-Cl (pH 7.4), 500 mM NaCl, 150 mM imidazole, 1 mM β-mercaptoethanol and ethylenediaminetetraacetic acid-free protease inhibitors (Sigma Aldrich, St. Louis, MO), and then washed with the same buffer. His-tagged recombinant Acot isoforms were then eluted from the HisTrap HP column using 20 mM Tris-Cl (pH 7.4), 500 mM NaCl, 500 mM imidazole, 1 mM β-mercaptoethanol and ethylenediaminetetraacetic acid-free protease inhibitors (Sigma Aldrich, St. Louis, MO). The soluble fraction containing MBP-tagged recombinant Acot9 was applied to a MBPTrap HP column after equilibration with 20 mM Tris-Cl, (pH 7.4) 200 mM NaCl, 1 mM EDTA and protease inhibitors (Sigma Aldrich, St. Louis, MO), washed with the same buffer and then eluted with 20 mM Tris-Cl (pH 7.4), 200 mM NaCl, 1 mM EDTA, 10 mM Maltose and protease inhibitors (Sigma Aldrich, St. Louis, MO). Following purification, all recombinant Acot isoforms were subjected to dialysis into buffer containing 20 mM Tris-Cl (pH 7.4), 500 mM NaCl and 10% glycerol using a slide-a-lyzer dialysis cassette (Thermo Fisher; Waltham, MA). Assessment of purity, protein concentration and storage of single use aliquots of recombinant Acot isoforms, and adjustment of buffer pH values were performed as described above for recombinant Them1.

Recombinant human Them1 START domain (START; amino acids 339-594 of Them1b) was prepared as previously described (Tillman et al. 2020). A synthetic gene encoding the START domain was cloned into a pNIC28-Bsa4 bacterial expression vector (Novagen, EMD Biosciences; Madison, WI), which introduced an in-frame N-terminal His₆ fusion containing a tobacco etch virus protease cleavage site to facilitate tag removal. The pNIC28-Bsa4-START domain plasmid was co-transformed into E. coli BL21 (DE3) (New England Biolabs; Ipswich, MA) competent cells with a pG-Tf2 vector (encoding groES-gorEL-tig chaperones) and then grown to an A₆₀₀ of 0.5-0.7 in terrific broth. Under conditions of shaking (250 rpm) at 18° C., chaperones were induced by the addition of 5 ng/mL tetracycline HCl for 60 min followed by START domain induction upon the addition of 0.5 mM isoproyl β-D-thiogalactoside for ˜18 h. The bacteria were harvested by centrifugation, and then lysed by sonication with 20 mM Tris-Cl (pH 7.4), 500 mM NaCl, 25 mM imidazole, 5% glycerol, lysozyme, Dnase A, 0.1% Triton X-100, 5 mM β-mercaptoethanol and 100 μM phenylmethylsulfonyl fluoride. Following centrifugation of the bacterial lysate, recombinant START was purified by FPLC using a His affinity column (HisTrap HP column; GE Healthcare; Waukesha, WI). The soluble fraction containing recombinant START was applied to the column after equilibration with 20 mM Tris-Cl (pH 7.4), 500 mM NaCl, 25 mM imidazole and 5% glycerol, and then washed with the same buffer. Recombinant START was eluted from the HisTrap HP column using 20 mM Tris-Cl (pH 7.4), 500 mM NaCl, 500 mM imidazole and 5% glycerol followed by tobacco etch virus protease-mediated His-tag cleavage at 4° C. overnight with simultaneous dialysis into 20 mM Tris-Cl (pH 7.4), 500 mM NaCl and 5% glycerol. Recombinant START was further purified using a Superdex column (HiLoad 16/60 Superdex 75 column; GE Healthcare; Waukesha, WI) in 20 mM Tris-Cl (pH 7.4), 500 mM NaCl and 5% glycerol. Assessment of purity, protein concentration and storage of single use aliquots of recombinant START were performed as described above for recombinant Them1.

### Kinetic Characterization of Acot Activity

Steady-state kinetic parameters were determined as previously described with modifications (Han and Cohen, 2012). Acot activity was determined in 384-well microplates for 60 min at 22° C. as a function of time after mixing recombinant Acot isoform (125 nM) with substrate C14-CoA (25 μM). Initial rates (V₀) were determined using GraphPad Prism (GraphPad Software; San Diego, CA). Concentrations of C14-CoA were varied to create saturation curves, and values of V₀ were fitted to the Michaelis-Menten equation using GraphPad Prism GraphPad Software; San Diego, CA). Nonlinear analysis of the Michaelis-Menten equation provided satisfactory curve-fits with an average R²=0.99 and a minimum R²>0.95. K_{cat} values were determined by fitting the calculated values of V₀ and substrate concentrations at 0, 15, 30, 45, 60 and 90 min timepoints to a sigmoidal concentration-response curve using GraphPad Prism GraphPad Software; San Diego, CA).

### Thermal Stability Assay

The thermal stability of Them1 was determined through a Thermal stability assay, which measures intrinsic changes in fluorescence from tryptophan and tyrosine residues at 350 nm and 330 nm, respectively that are exposed as a protein unfolds. The assay was performed in Tyco NT.6 capillaries using a Tyco NT.6 system (NanoTemper; San Francisco, CA). Reactions consisting of Them1 (5 μM) in Assay buffer (Thermo Fisher; Waltham, MA) were added to each capillary. Values of fluorescence intensity were determined in 1° C. increments (40 to 80° C.) measured at 350 nm and 330 nm. Values of inflection temperature (Tᵢ) were determined using the Tyco NT.6 software (NanoTemper; San Francisco, CA), which identified Tᵢ as minimum values of −dF/dT when plotted as functions of temperature.

### Assay for Reversibility of Them1 Inhibition

Them1 activity was first measured in reactions consisting of recombinant Them1 (125 nM), C14-CoA (25 μM) and compounds (125 μM) incubated in 384-well microplates for 60 min at 22° C. Reactions were dialyzed at 4° C. in buffer containing 20 mM Tris-Cl (pH 7.4) and 500 mM NaCl using slide-a-lyzer dialysis cassettes (Thermo Fisher; Waltham, MA) with the pH adjusted to remain at least 2 units removed from the pI of recombinant Them1. Three buffer changes were performed over the extent of the 18 h dialysis. Post-dialysis reactions were incubated with fresh C14-CoA (25 μM) for 60 min at 22° C. Ebselen (Sigma Aldrich; St. Louis, MO) was used as a (−) control.

Reversibility of inhibition was confirmed by dialysis for all compounds (FIGS. 5B, 12, and 13).

### Microscale Thermophoresis

Reaction labeling recombinant full-length Them1, Them1l-ΔSTART, START or Acot12 with Monolith RED-Tris-NTA (NanoTemper Technologies; San Francisco, CA) were performed according to the manufacturer's instructions in buffer containing PBS plus 0.5% Tween-20 for 60 min at 22° C. with a molar dye: protein ratio=1:1. Compounds starting at 125 μM were serially diluted in DMSO. Working concentrations of compounds were prepared by diluting in microscale thermophoresis buffer. Reactions consisting of 100 nM of fluorescently labeled recombinant protein (full-length Them1, Them1-ΔSTART, START or Acot12) and compounds were loaded onto standard Monolith NT.115 Capillaries (NanoTemper Technologies; San Francisco, CA). Fluorescence was measured using a Monolith NT.115 instrument (NanoTemper Technologies; San Francisco, CA) at 22° C. with instrument parameters adjusted to 40% LED power and medium microscale thermophoresis power. Data were fitted by non-linear regression in GraphPad Prism (GraphPad Software; San Diego, CA).

### Cytotoxicity Assay

Cytotoxicities of compounds were determined using a CellTiter-Glo® Luminescent Cell Viability Assay, which measures concentrations of ATP liberated from viable cells (Promega; Madison, WI). Primary brown adipocytes cultured from C57BL/6 mice were seeded at 2,000 per well into white 384-well transparent bottom opaque microplates (GreinerBio One International AG; Kremsmünster, Austria) pre-coated with rat tail collagen (Sigma Aldrich; St. Louis, MO) at 37° C. and treated for 48 h with DMSO (Control) or compounds (250 μM to 7.7 nM) in growth medium. Tamoxifen (50 μM) was used as a (+) control. CellTiter-Glo reagent (10 μL) was added to each well and incubated on an orbital shaker for 10 min at 22° C. to induce cell lysis and stabilize the luminescent signal. Relative luminescent units (RLU) were determined using a NEO plate reader (BioTek, Winooski, VT).

### Critical Aggregation Concentration

Critical aggregation concentrations of compounds were determined using dynamic light scattering in black 96-well flat bottom polystyrene microplates (GreinerBio One International AG; Kremsmünster, Austria) at 22° C. using a DynaPro plate reader (Wyatt Technology; Santa Barbara, CA). 50 mM stock compounds dissolved in pure DMSO were serial diluted 2-fold (250 to 62.5 μM) in PBS and then spun down at 14,000×g for 10 min at 22° C. followed by filtration through a 0.22 μm filter unit. Aggregation of compounds were assessed by measuring the amplitude of the autocorrelation intensity calculated from the average of 3 to 5 acquisitions per replicate.

Each of these compounds exhibited a critical aggregation concentration >125 μM, with the exception of U15 at 122 μM. These high values negated the possibility Them1 inhibition might have attributable to compound aggregation (FIG. 14).

### Oxygen Consumption Rates (OCR)

### Oxygen Consumption Rates (OCR)

In primary brown adipocytes on d 8 of differentiation, values of OCR were measured in the presence or absence of compounds using a Seahorse XF96 extracellular flux analyzer (Agilent Technologies; Santa Clara, CA). Primary brown adipocytes were treated for 30 min with DMSO (Control) or compounds in serum-free growth medium followed by incubation in the absence of CO₂ for 60 min at 37° C. in Krebs-Henseleit buffer (pH 7.4) containing 0.45 g/L glucose, 111 mM NaCl, 4.7 mM KCl, 2 mM MgSO₄⁻7H₂O, 1.2 mM Na₂HPO₄, 5 mM HEPES and 0.5 mM carnitine (Sigma-Aldrich; St. Louis, MO). OCR values were measured before and after the exposure of cells to NE (1 μM) and normalized with total live cell count calculated through staining with NucRed Live probe (Thermo Fisher; Waltham, MA) measured at 715 nm emission and 623 nm excitation using a SpectraMax i3X plate reader (Molecular Devices; San Jose; CA).

In primary hepatocytes, values of OCR were measured using a Seahorse XF24 extracellular flux analyzer (Agilent Technologies; Santa Clara, CA). Following 4 to 5 h of incubation in William's Medium E (Thermo Fisher; Waltham, MA) supplemented with 10% FBS and 1% penicillin-streptomycin, primary hepatocytes were treated for 30 min with DMSO (Control) or compounds in serum-free Medium 199 (Thermo Fisher; Waltham, MA) followed by incubation in the absence of CO₂ for 60 min at 37° C. in Krebs-Henseleit buffer (pH 7.4) containing 0.45 g/L glucose, 111 mM NaCl, 4.7 mM KCl, 2 mM MgSO₄⁻H₂O, 1.2 mM Na₂HPO₄, 5 mM HEPES and 0.5 mM carnitine (Sigma-Aldrich; St. Louis, MO). OCR values were measured before and after the exposure of cells to palmitic acid conjugated with fatty acid-free BSA (300 μM) and normalized with total live cell count calculated through staining with NucRed Live probe (Thermo Fisher; Waltham, MA) measured at 715 nm emission and 623 nm excitation using a SpectraMax i3X plate reader (Molecular Devices; San Jose; CA).

### RNA Extraction and Analysis of Gene Expression

Total RNA was extracted from mouse WAT, BAT and kidney and primary brown adipocytes cultured from Control and A-Them1Tg mice using QIAzol lysis reagent (Qiagen; Valencia, CA), and used to synthesize cDNA with a High-Capacity cDNA Reverse Transcription Kit (Applied Biosystems; Foster City, CA). Gene expression was analyzed with quantitative real-time PCR assays using Power SYBR Green Mix (Applied Biosystems; Foster City, CA). Real-time PCR assays were performed in triplicate with a total reaction volume of 25 μL containing 500 nM concentrations of each primer and cDNA (25 ng). mRNA expression levels were normalized to the housekeeping gene Actβ or 18S.

### Glucose Production

Rates of glucose production were determined as previously described with modifications (Kawano et al., 2014). Primary hepatocytes cultured from wild type, L-Them1Tg and Them1^{-/'} mice were serum-starved in Medium 199 (Thermo Fisher; Waltham, MA) for 16 h and then washed twice with PBS. Cells were then incubated for 6 h in glucose-, L-glutamine- and phenol red-free DMEM (Thermo Fisher; Waltham, MA) supplemented with compounds, 2 mM sodium pyruvate and 20 mM sodium lactate. Glucose concentrations in the media were measured enzymatically through a commercial kit according to the manufacturer's instructions using a NEO plate reader (BioTek, Winooski, VT).

### Immunoblot Analysis

Tissue extracts from mouse brain, heart, lung, WAT, skeletal muscle (muscle) and kidney, and total cellular extracts from primary brown adipocytes were prepared in 10 mM Tris-HCl (pH 7.4), 150 mM NaCl, 1% Nonidet P-40, 1 mM phenylmethylsulfonyl fluoride, 1 mM EDTA, 1 mM NaF, 0.25% sodium deoxycholate, and 10% glycerol, supplemented with protease and phosphatase inhibitors (Thermo Fisher; Waltham, MA). Protein extracts were separated on 10 to 12% polyacrylamide gels and transferred onto nitrocellulose membranes (Protan, Schleicher, and Schuell Bioscience; Dassel, Germany). Membranes were blocked in Tris-buffered saline with Tween-20 (0.05 M Tris-HCl (pH 7.4), 0.2 M NaCl, and 0.1% Tween-20) containing (5% wt/vol) nonfat dried skim milk. Membranes were then immunodecorated with primary antibodies against mouse Them1 (Han and Cohen, 2012; Zhang et al., 2012), FLAG (BioLegend; San Diego, CA) and Hsp90 (Santa Cruz Biotechnology; Santa Cruz, CA), and diluted in blocking solution. Signals were developed with goat anti-rabbit or goat anti-rat secondary antibodies (Thermo Fisher; Waltham, MA), and visualized with the ProteinSimple system (ProteinSimple; San Jose, CA).

### Quantification and Statistical Analysis

Data were analyzed by a mixed model using the fit model procedure of JMP Pro 11.0 satistical software (SAS Institute; Cary, NC). For experiments measuring OCR in primary brown adipocytes cultured from Control and A-Them1Tg mice and corresponding values of area under the curve (AUC), data were analyzed by two-way ANOVA accounting for genotype and compound. For analyses measuring Them1 mRNA abundance in human tissues (BAT, WAT and liver), data were analyzed by pair-wise comparisons accounting for physiological state or tissue). For the reversibility washout experiments, data were analyzed by pair-wise comparisons accounting for dialysis. For experiments measuring Them1 mRNA abundance in tissues from Control and A-Them1Tg mice, data were analyzed by one-way ANOVA accounting for genotype. For Them1 mRNA abundance in primary brown adipocytes, data were analyzed by pair-wise comparisons accounting for genotype. Statistical significance was determined using two-tailed unpaired Student t-test or two-way ANOVA followed by Tukey's post-hoc test for comparisons between two or three groups. Differences were considered significant at P<0.05. All statistical analyses were performed using JMP Pro 11.0 statistical software.

### Acot Activity Assay

A 96-well fluorometric Acot activity assay was developed using a commercial kit detecting free thiols (Thermo fisher; Waltham, MA), and then scaled down and optimized in polystyrene 384-well microplates. Free CoA liberated by Acot activity covalently binds to a non-fluorescent detection reagent, resulting in the generation of a fluorescent product. Upon the addition of C14-CoA, time-dependent increases in fluorescence intensity reflected free CoA covalently bound to detection reagent.

To determine IC₅₀ values, compounds were serially diluted starting at 20 μM in DMSO for a total of 10 dilutions. Compounds exhibiting IC₅₀ values ≤20 μM were further tested for selectivity using a counter screen against other Acot isoforms (i.e. Acot1, Acot2, Acot9 and Acot13), as well as Them1-ΔSTART. Compound IC₅₀ values derived from the Acot activity assay were calculated using GraphPad Prism (San Diego, CA) from triplicate determinations. The Z′ factor, which is a measure of suitability of an assay for HTS, was calculated as Z′(60′)=1-3(S.D.₋+S.D.₊)/[F(60′)₋-F(60′)₊] (Zhang et al., 1999). Values are show in the following Table.

| Compound | Structure | IC50 (μM) |
|---|---|---|
| U1 | 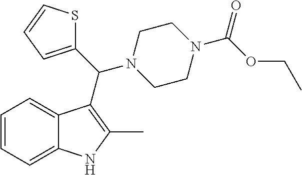 [SMILES: CCOC(=O)N1CCN(C(c2cccs2)c2c(C)[nH]c3ccccc23)CC1] | 32.9 ± 1.14 |
| U2 | 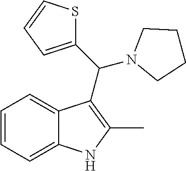 [SMILES: Cc1[nH]c2ccccc2c1C(c1cccs1)N1CCCC1] | 8.66 ± 1.05 |
| U3 | 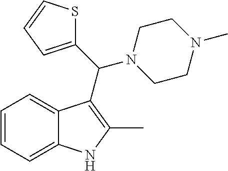 [SMILES: Cc1[nH]c2ccccc2c1C(c1cccs1)N1CCN(C)CC1] | 8.60 ± 1.03 |
| U4 | 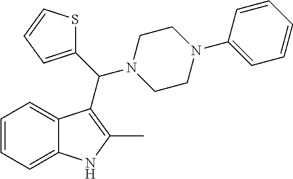 [SMILES: Cc1[nH]c2ccccc2c1C(c1cccs1)N1CCN(c2ccccc2)CC1] | 6.29 ± 1.12 |
| U5 | 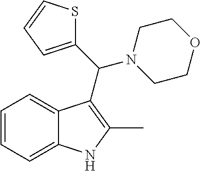 [SMILES: Cc1[nH]c2ccccc2c1C(c1cccs1)N1CCOCC1] | 7.63 ± 1.12 |
| U6 | 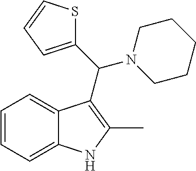 [SMILES: Cc1[nH]c2ccccc2c1C(c1cccs1)N1CCCCC1] | 35.4 (1.13) |
| U7 | 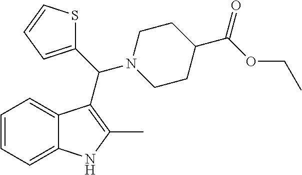 [SMILES: CCOC(=O)C1CCN(C(c2cccs2)c2c(C)[nH]c3ccccc23)CC1] | 78.5 (1.65) |
| U8 | 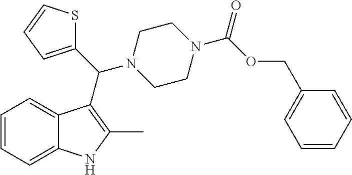 [SMILES: Cc1[nH]c2ccccc2c1C(c1cccs1)N1CCN(C(=O)OCc2ccccc2)CC1] | >125 |
| U9 | 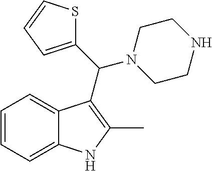 [SMILES: Cc1[nH]c2ccccc2c1C(c1cccs1)N1CCNCC1] | 46.8 ± 2.26 |
| U10 | 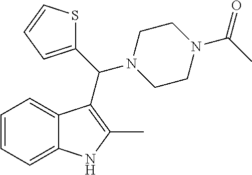 [SMILES: CC(=O)N1CCN(C(c2cccs2)c2c(C)[nH]c3ccccc23)CC1] | 24.4 ± 1.10 |
| U11 | 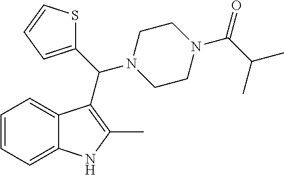 [SMILES: Cc1[nH]c2ccccc2c1C(c1cccs1)N1CCN(C(=O)C(C)C)CC1] | 3.63 ± 1.05 |
| U12 | 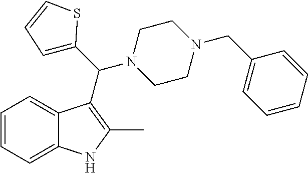 [SMILES: Cc1[nH]c2ccccc2c1C(c1cccs1)N1CCN(Cc2ccccc2)CC1] | 8.53 ± 1.11 |
| U13 | 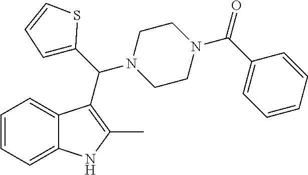 [SMILES: Cc1[nH]c2ccccc2c1C(c1cccs1)N1CCN(C(=O)c2ccccc2)CC1] | 20.0 ± 1.10 |
| U14 | 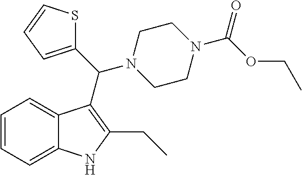 [SMILES: CCOC(=O)N1CCN(C(c2cccs2)c2c(CC)[nH]c3ccccc23)CC1] | 8.49 ± 1.07 |
| U15 | 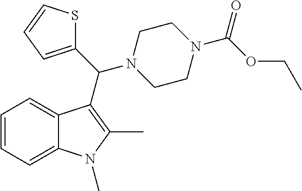 [SMILES: CCOC(=O)N1CCN(C(c2cccs2)c2c(C)n(C)c3ccccc23)CC1] | 32.6 ± 1.19 |
| U16 | | 41.9 ± 1.21 |
| U17 | | >125 |

Compound U1 bound more tightly to the START domain (K_{d}: 1.03 μM) compared to full-length Them1 (K_{d}: 8.00 μM) and lacked binding to Them1-ΔSTART (FIGS. 10A-D). SAR-dependent differences in IC₅₀ values relative to the parent U1 compound were associated with tighter (e.g., U11, U12 and U14), comparable (e.g., U7, U13 and U16) or negligible (e.g., U8) binding affinities to full-length Them1 and the START domain, but consistently did not bind Them1-ΔSTART. In further support as a START domain-dependent inhibitor selective for Them1, the parent U1 compound and its structural derivatives lacked the capacity to bind Acot12, a related Acot isoform also containing a C-terminal START domain.

| | | | Thioesterase domains | |
|---|---|---|---|---|
| Compound | Them1 | START | (Them1-ΔSTART ) | Acot12 |
| U1 | 8.00 (2.36) | 1.03 (0.78) | >125 | >125 |
| U6 | 41.8 (16.4) | 40.7 (15.6) | >125 | >125 |
| U7 | 9.21 (4.42) | 17.3 (7.69) | >125 | >125 |
| U8 | >125 | >125 | >125 | >125 |
| U9 | 0.52 (0.23) | 21.1 (2.64) | >125 | >125 |
| U10 | 16.2 (5.20) | 5.44 (1.81) | >125 | >125 |
| U11 | 0.87 (0.21) | 0.30 (0.07) | >>125 | >125 |
| U12 | 0.18 (0.07) | 0.17 (0.09) | >>125 | >125 |
| U13 | 6.36 (1.75) | 0.19 (0.07) | >125 | >125 |
| U14 | 3.39 (1.92) | 4.12 (1.48) | >125 | >125 |
| U15 | 1.05 (1.34) | 3.56 (2.22) | >125 | >125 |
| U16 | 4.19 (1.24) | 1.00 (0.58) | >125 | >125 |
| U17 | 16.4 (6.67) | 6.48 (3.11) | >125 | >125 |

The potency of structural derivatives of the parent U1 compound were tested at their in vitro IC₅₀ concentrations using the brown adipocyte-based OCR assay. A-Them1Tg brown adipocytes treated with structural derivatives (i.e., U6, U9, U11 and U12) reversed the suppressive effects of Them1 on values of OCR compared to wild type brown adipocytes treated with DMSO (FIGS. 11A-C). By contrast, OCR values in A-Them1Tg brown adipocytes remained suppressed upon treatment with compounds U8, U14 and U15 (FIGS. 11D-E). The possibility of compound cellular toxicity was discounted by demonstrating that compounds U1 structural derivatives exhibited half-maximal cellular lethal concentrations (LC₅₀)>125 μM (FIG. 15).

### INCORPORATION BY REFERENCE

All publications and patents mentioned herein are hereby incorporated by reference in their entirety as if each individual publication or patent was specifically and individually indicated to be incorporated by reference. In case of conflict, the present application, including any definitions herein, will control.

### EQUIVALENTS

While specific embodiments of the subject disclosure have been discussed, the above specification is illustrative and not restrictive. Many variations of the disclosure will become apparent to those skilled in the art upon review of this specification and the claims below. The full scope of the disclosure should be determined by reference to the claims, along with their full scope of equivalents, and the specification, along with such variations.

### REFERENCES

Adams, S. H., Chui, C., Schilbach, S. L., Yu, X. X., Goddard, A. D., Grimaldi, J. C., Lee, J., Dowd, P., Colman, S., and Lewin, D. A. (2001). BFIT, a unique acyl-CoA thioesterase induced in thermogenic brown adipose tissue: cloning, organization of the human gene and assessment of a potential link to obesity. Biochem. J. 360, 135-142.
Blither, M. (2019). Obesity: global epidemiology and pathogenesis. Nat. Rev. Endocrinol. 2019 155 15, 288-298.
Cannon, B., and Nedergaard, J. (2004). Brown Adipose Tissue: Function and Physiological Significance. Physiol. Rev. 84, 277-359.
Costa-Mattioli, M., and Walter, P. (2020). The integrated stress response: From mechanism to disease. Science 368.
Desai, A., Alves-Bezerra, M., Li, Y., Ozdemir, C., Bare, C. J., Li, Y., Hagen, S. J., and Cohen, D. E. (2018). Regulation of fatty acid trafficking in liver by thioesterase superfamily member 1. J. Lipid Res. 59, 368-379.
Ellis, J. M., Li, L. O., Wu, P.-C., Koves, T. R., Ilkayeva, O., Stevens, R. D., Watkins, S. M., Muoio, D. M., and Coleman, R. A. (2010). Adipose acyl-CoA synthetase-1 directs fatty acids toward beta-oxidation and is required for cold thermogenesis. Cell Metab. 12, 53-64.
Ellis, J. M., Bowman, C. E., and Wolfgang, M. J. (2015). Metabolic and tissue-specific regulation of acyl-CoA metabolism. PLoS One 10.
Goh, L. L., Lee, Y., Tan, E. S., Lim, J. S. C., Lim, C. W., and Dalan, R. (2018). Patient with multiple acyl-CoA dehydrogenase deficiency disease and ETFDH mutations benefits from riboflavin therapy: a case report. BMC Med. Genomics 11.
Han, S., and Cohen, D.E. (2012). Functional characterization of thioesterase superfamily member 1/Acyl-CoA thioesterase 11: implications for metabolic regulation. J. Lipid Res. 53, 2620-2631.
Holm, C. (2003). Molecular mechanisms regulating hormone-sensitive lipase and lipolysis. In Biochemical Society Transactions, (Portland Press Ltd), pp. 1120-1124.
Hunt, M. C., Yamada, J., Maltais, L. J., Wright, M. W., Podesta, E. J., and Alexson, S. E. H. (2005). A revised nomenclature for mammalian acyl-CoA thioesterases/hydrolases. J. Lipid Res. 46, 2029-2032.
Kazankov, K., Jorgensen, S. M. D., Thomsen, K. L., Moller, H. J., Vilstrup, H., George, J., Schuppan, D., and GrØbæk, H. (2019). The role of macrophages in nonalcoholic fatty liver disease and nonalcoholic steatohepatitis. Nat. Rev. Gastroenterol. Hepatol. 16, 145-159.
Loomba, R., Friedman, S. L., and Shulman, G. I. (2021). Mechanisms and disease consequences of nonalcoholic fatty liver disease. Cell 184, 2537-2564.
Okada, K., LeClair, K. B., Zhang, Y., Li, Y., Ozdemir, C., Krisko, T. I., Hagen, S. J., Betensky, R. A., Banks, A. S., and Cohen, D. E. (2016). Thioesterase superfamily member 1 suppresses cold thermogenesis by limiting the oxidation of lipid droplet-derived fatty acids in brown adipose tissue. Mol. Metab. 5, 340-351.
Schrick, K., Nguyen, D., Karlowski, W. M., and Mayer, K. F. X. (2004). START lipid/sterol-binding domains are amplified in plants and are predominantly associated with homeodomain transcription factors. Genome Biol. 5.
Singh, J., Petter, R. C., Baillie, T. A., and Whitty, A. (2011). The resurgence of covalent drugs. Nat. Rev. Drug Discov. 2011 104 10, 307-317.
Sztalryd, C., and Kimmel, A. R. (2014). Perilipins: lipid droplet coat proteins adapted for tissue-specific energy storage and utilization, and lipid cytoprotection. Biochimie 96, 96-101.
Tillander, V., Nordstrom, E .A., Reilly, J., Strozyk, M., Van Veldhoven, P. P., Hunt, M. C., and Alexson, S .E .H. (2014). Acyl-CoA thioesterase 9 (ACOT9) in mouse may provide a novel link between fatty acid and amino acid metabolism in mitochondria. Cell. Mol. Life Sci. 71, 933-948.
Tillander, V., Alexson, S. E. H., and Cohen, D. E. (2017). Deactivating Fatty Acids: Acyl-CoA Thioesterase-Mediated Control of Lipid Metabolism. Trends Endocrinol. Metab. 28, 473-484.
Tillman, M. C., Imai, N., Li, Y., Khadka, M., Okafor, C.D ., Juneja, P., Adhiyaman, A., Hagen, S. J., Cohen, D. E., and Ortlund, E.A. (2020). Allosteric regulation of Thioesterase Superfamily Member 1 by free fatty acids and lysophosphatidylcholine. BioRxiv 2020.02.18.954917.
Xu, X., Krumm, C., So, J. S., Bare, C. J., Holman, C., Gromada, J., Cohen, D. E., and Lee, A. H. (2018). Preemptive Activation of the Integrated Stress Response Protects Mice From Diet-Induced Obesity and Insulin Resistance by Fibroblast Growth Factor 21 Induction. Hepatology 68, 2167-2181.
Younossi, Z. M., Koenig, A. B., Abdelatif, D., Fazel, Y., Henry, L., and Wymer, M. (2016). Global epidemiology of nonalcoholic fatty liver disease—Meta-analytic assessment of prevalence, incidence, and outcomes. Hepatology 64, 73-84.
Zhang, J.-H., Chung, and Oldenburg (1999). A Simple Statistical Parameter for Use in Evaluation and Validation of High Throughput Screening Assays. J. Biomol. Screen. 4, 67-73.
Zhang, Y., Li, Y., Niepel, M .W., Kawano, Y., Han, S., Liu, S., Marsili, A., Larsen, P. R., Lee, C.-H., and Cohen, D. E. (2012). Targeted deletion of thioesterase superfamily member 1 promotes energy expenditure and protects against obesity and insulin resistance. Proc. Natl. Acad. Sci. U. S. A. 109, 5417-5422.

1. A compound of formula (I): 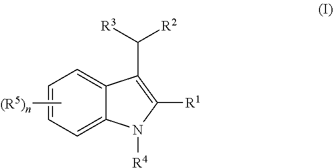
or a pharmaceutically acceptable salt thereof, wherein:
R¹ is H, alkyl, alkenyl, alkynyl, aryl, heteroaryl, cycloalkyl, heterocycloalkyl, halogen, cyano, hydroxy, alkoxy, amino, acylamino, amide, acyl, acyloxy, carboxy, ester, sulfonamide, sulfone;
R² is aryl, heteroaryl, cycloalkyl, cycloalkenyl, or heterocycloalkyl;
R³ is aryl, heteroaryl, cycloalkyl, cycloalkenyl, or heterocycloalkyl;
R⁴ is H, alkyl, alkenyl, or alkynyl,
R⁵ is halogen, alkyl, or cycloalkyl, and
n is 0-4.

2. The compound of claim 1, wherein the compound is of formula (II): 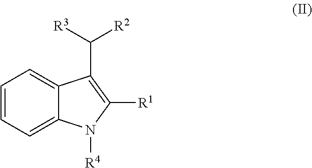
or a pharmaceutically acceptable salt thereof.

3. The compound of claim 1, wherein R² is heterocycloalkyl.

4. The compound of claim 3, wherein the heterocycloalkyl comprises at least one nitrogen atom.

5. The compound of claim 1, wherein the compound is of formula (III) 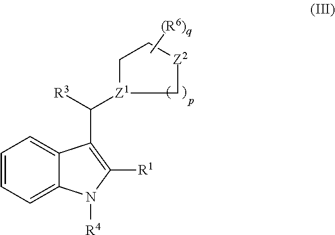
or a pharmaceutically acceptable salt thereof, wherein:
Z¹ is N or CR^{a};
Z² is O, NR^{b} or CR^{c}R^{d};
each R^{a}, R^{b}, R^{c}, and R^{d} is independently H, alkyl, alkenyl, alkynyl, aryl, heteroaryl, cycloalkyl, heterocycloalkyl, halogen, cyano, hydroxy, alkoxy, amino, acylamino, amide, acyl, acyloxy, ester, sulfonamide, sulfone;
each R⁶ is alkyl, alkenyl, alkynyl, aryl, heteroaryl, cycloalkyl, heterocycloalkyl, halogen, cyano, hydroxy, alkoxy, amino, acylamino, amide, acyl, acyloxy, ester, sulfonamide, sulfone;
p is 1 or 2; and
q is 0 to 4.

6. The compound of claim 5, wherein Z¹ is CH.

7. The compound of claim 5, wherein Z¹ is N.

8. The compound of claim 5, wherein Z² is NR^{b}.

9. (canceled)

10. The compound of claim 8, wherein R^{b} is hydrogen, C₁-C₆ alkyl, —C(O)C₁-C₆ alkyl, or —C(O)₂C₁-C₆ alkyl.

11. The compound of claim 10, wherein R^{b} is substituted with cycloalkyl, heterocycloalkyl, aryl such as phenyl, or heteroaryl.

12. The compound of claim 5, wherein Z² is CR^{c}R^{d} or O.

13. (canceled)

14. The compound of claim 1, wherein the compound is of formula (IV) 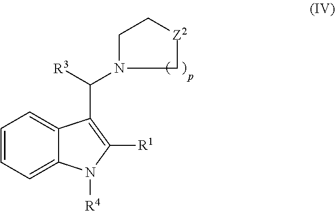
or a pharmaceutically acceptable salt thereof, wherein:
Z² is O, NR^{b} or CR^{c}R^{d};
each of R^{b}, R^{c}, and R^{d} is independently H, —C(O)OC₁-C₆ alkyl, —C(O)C₁-C₆ alkyl, or —C(O)-aryl, wherein C₁-C₆ alkyl is optionally substituted by aryl; and
p is 1 or 2.

15. The compound of claim 14, wherein the compound is of formula (V) 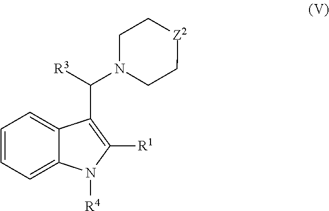
or a pharmaceutically acceptable salt thereof, wherein:
Z² is O, NR^{b} or C(H)R^{c}; and
each of R^{b} and R^{c} is independently H, —C(O)OC₁-C₆ alkyl, —C(O)C₁-C₆ alkyl, or —C(O)-aryl, e.g., wherein each of R^{b} and R^{c} is optionally substituted by aryl.

16.-20. (canceled)

21. The compound of claim 1, wherein R³ is heteroaryl.

22. The compound of claim 1, wherein R³ is thiopheneyl, and wherein the thiopheneyl is optionally substituted by alkyl such as C₁-C₆ alkyl.

23.-25. (canceled)

26. The compound of claim 1, wherein R¹ is alkyl such as C₁-C₆ alkyl

27.-29. (canceled)

30. The compound of claim 1, wherein R⁴ is H or alkyl.

31.-33. (canceled)

34. The compound of claim 1, wherein the compound is not

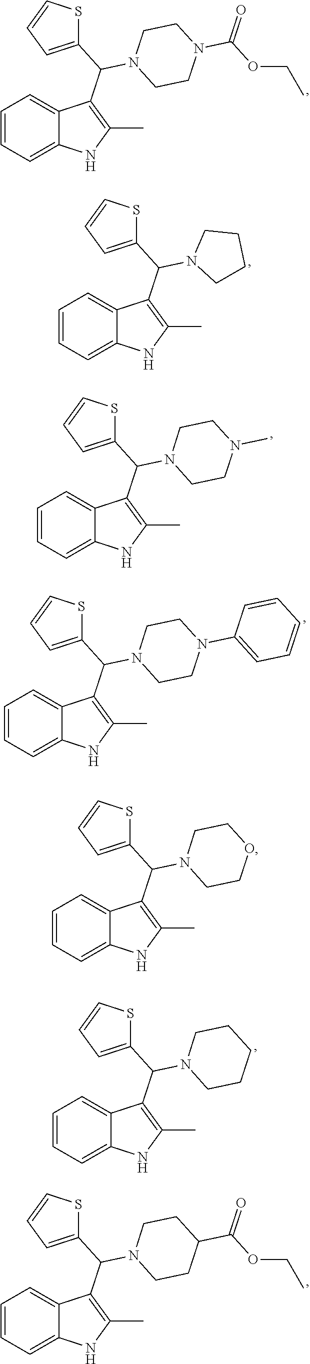

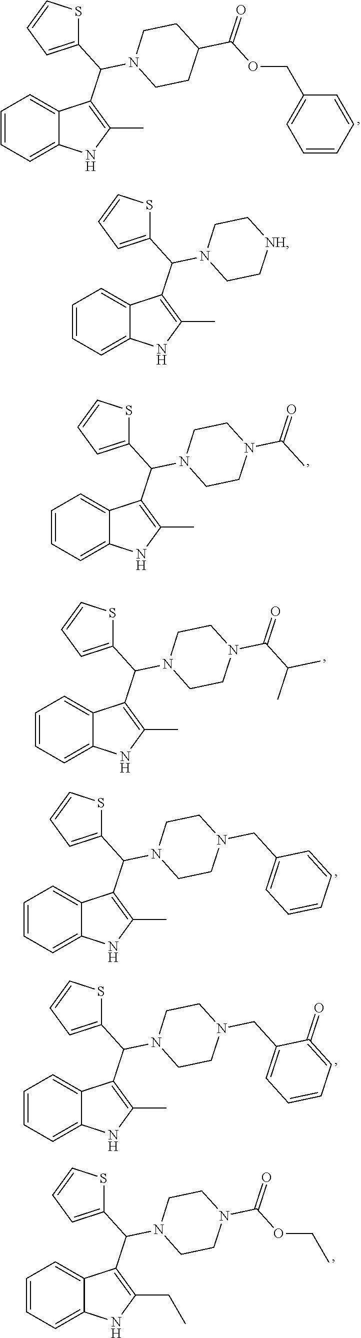

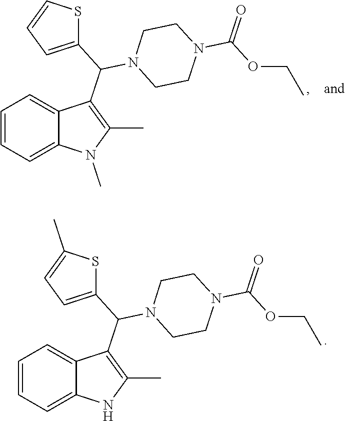 [SMILES: CCOC(=O)N1CCN(C(c2cccs2)c2c(C)n(C)c3ccccc23)CC1 ; CCOC(=O)N1CCN(C(c2ccc(C)s2)c2c(C)[nH]c3ccccc23)CC1]

35. (canceled)

36. A pharmaceutical composition comprising a compound of claim 1 and one or more pharmaceutically acceptable excipients

37. A method of treating a metabolic disease or a cardiovascular disease, the method comprising administering a therapeutically effective amount of a compound according to claim 1 to a subject in need thereof.

38.-40. (canceled)
